(19) United States
(12) Patent Application Publication
(10) Pub. No.: US 2006/0135506 A1
(43) Pub. Date: Jun. 22, 2006

(54) Compositions of a cyclooxygenase-2 selective inhibitor and a calcium modulating agent for the treatment of pain, inflammation or inflammation mediated disorders

(75) Inventors: Diane T. Stephenson, Groton, CT (US); Duncan P. Taylor, Bridgewater, NJ (US)

Correspondence Address: SENNIGER POWERS, ONE METROPOLITAN SQUARE, 16TH FLOOR, ST LOUIS, MO, 63102, US

(73) Assignee: Pharmacia Corporation

(21) Appl. No.: 10/829,000

(22) Filed: Apr. 21, 2004

Related U.S. Application Data
(60) Provisional application No. 60/464,609, filed on Apr. 22, 2003.

(51) Int. Cl. A61K 31/553; A61K 31/195; A61K 31/415; A61K 31/365

(52) U.S. Cl. 514/211.07; 514/406; 514/562; 514/471; 514/355

(57) ABSTRACT

The present invention provides compositions and methods for the treatment of pain, inflammation or inflammation mediated disorders in a subject. More particularly, the invention provides a combination therapy for the treatment of pain, inflammation or inflammation mediated disorders comprising the administration to a subject of a calcium modulating agent in combination with a cyclooxygenase-2 selective inhibitor.

### CROSS REFERENCE TO RELATED APPLICATION

This application claims priority from Provisional Application: Ser. No. 60/464,609 filed on Apr. 22, 2003, which is hereby incorporated by reference in its entirety.

### FIELD OF THE INVENTION

The present invention provides methods and compositions related to the treatment of pain, inflammation or inflammation mediated disorders. More particularly, the invention is directed toward a combination therapy for the treatment of pain, inflammation or inflammation mediated disorders comprising the administration to a subject of a calcium modulating agent in combination with a cyclooxygenase-2 selective inhibitor.

### BACKGROUND OF THE INVENTION

Pain is a sensory experience distinct from sensations of touch, pressure, heat and cold. It is often described by sufferers by such terms as bright, dull, aching, pricking, cutting or burning and is generally considered to include both the original sensation and the reaction to that sensation. Pain sensation is complex and variable. Often experiences considered painful by one subject may not be equally painful to another and may vary in the same subject depending on the circumstances presented. This range of sensations, as well as the variation in perception of pain by different individuals, renders a precise definition of pain difficult, however, many individuals suffer with severe and continuous pain.

Pain can be caused by the stimulation of nociceptive receptors and transmitted over intact neural pathways, in which case the pain is termed “nociceptive” pain. Generally speaking, there are two different types of nociceptive stimuli that are intense enough to be perceived as pain. One type, somatic pain, consists of an intense, localized, sharp or stinging sensation. Somatic pain is mediated by fast-conducting, lightly myelinated A-delta fibers that have a high threshold (i.e. require a strong mechanical stimulus to sense pain) and enter into the spinal cord through the dorsal horn of the central nervous system where they terminate in the spinal cord.

The second type of pain, sometimes referred to as visceral pain, is characterized as a diffuse, dull, aching or burning sensation. Visceral pain is mediated largely by unmyelinated, slower-conducting C-fibers that are polymodal (i.e., mediate mechanical, thermal, or chemical stimuli). C-fibers also enter the spinal cord through the dorsal horn of the central nervous system where they terminate in the spinal cord. Both somatic and visceral pain can be sensed centrally and peripherally within the human body and may be either acute or chronic.

A number of analgesics reduce both central and peripheral sensitization through interaction with the various pain-based receptors within the human body. For example, morphine and most other opioid analgesics elicit an inhibitory neuronal effect within central nervous and gastrointestinal (GI) systems by interacting with areas of the brain receiving input from the spinal pain-transmitting pathways containing opioid receptors. By suppressing neuronal activity at these receptor points, opioid narcotics produce analgesia and control the pain threshold within a human patient.

Opioid narcotics, however, have several negative side effects that severely limit their therapeutic value. These side effects include drowsiness, lethargy, difficulty in being mobile, respiratory depression, excessive central nervous system depression, weakness in the extremities, and dizziness. In addition, patients being treated with opioids also may develop tolerance to the agent, requiring higher doses, or addition of other opioids to the pain treatment regimen. The larger effective dosage may in turn lead to the development of physical and psychological addiction. Further, other typical side effects of opioid analgesics include miosis, or constriction of the pupils, nausea, vomiting, prolongation of stomach emptying time, and decreased propulsive contractions of the small intestine.

The analgesic effect of opioids may be enhanced by the simultaneous administration of calcium channel antagonists. Calcium channel antagonists are usually employed for the treatment of cardiovascular disease conditions such as high blood pressure, arrhythmia or angina pectoris. Due to the enhancement of the anti-nociceptive effect of opioids by means of calcium channel antagonists, lower doses of the opioid can be administered for the same analgesic effect.

As an alternative to opioid analgesics, a number of non-narcotic based drugs may be utilized to treat mild to moderate pain. Generally speaking, non-narcotic drugs can be given over longer periods of time compared to opioid analgesics because of their lower central nervous system and respiratory depressive effects. Examples of non-narcotic drugs employed to treat pain include acetylsalicylic acid (aspirin), centrally acting alpha antiadrenergic agents, diflusinal, salsalate, acetaminophen, and nonsteroidal anti-inflammatory agents such as ibuprofen, naproxen, and fenoprofen. These agents all generally relieve pain through prostaglandin synthesis inhibition resulting in a decrease in pain receptor stimulation.

Non-narcotic drugs also have several negative side effects that severely limit their therapeutic value. Aspirin, for example, has been shown through epidemiological data to be a factor in the occurrence of Reye's syndrome. In addition, salicylates have been shown to cause gastrointestinal upset, gastrointestinal hemorrhage, and anti-platelet effects. Acetaminophen has been linked to liver damage, kidney damage, and hematological effects such as hemolytic anemia, neutropenia, and leukopenia. Moreover, nonsteroidal anti-inflammatory agents also exhibit numerous negative side effects as well, ranging from gastrointestinal distress, gastrointestinal hemorrhage, and kidney damage when administered at a therapeutically effective dosage for the treatment of pain.

### SUMMARY OF THE INVENTION

Among the several aspects of the invention is provided a method and a composition for the treatment of pain, inflammation or inflammation-mediated disorders in a subject. The composition comprises a cyclooxygenase-2 selective inhibitor or an isomer, a pharmaceutically acceptable salt, ester, or prodrug thereof, and a calcium modulating agent and the method comprises administering the composition to a subject.

In one embodiment, the cyclooxygenase-2 selective inhibitor is a member of the chromene class of compounds. For example, the chromene compound may be a compound of the formula:

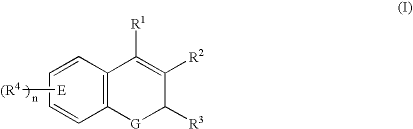

wherein:

n is an integer which is 0, 1, 2, 3 or 4;

G is O, S or NR^{a};

R^{a} is alkyl;

R¹ is selected from the group consisting of H and aryl;

R² is selected from the group consisting of carboxyl, aminocarbonyl, alkylsulfonylaminocarbonyl and alkoxycarbonyl;

R³ is selected from the group consisting of haloalkyl, alkyl, aralkyl, cycloalkyl and aryl optionally substituted with one or more radicals selected from alkylthio, nitro and alkylsulfonyl; and

each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;

or wherein R⁴ together with the carbon atoms to which it is attached and the remainder of ring E forms a naphthyl radical,

or prodrug thereof.

In another embodiment, the cyclooxygenase-2 selective inhibitor or an isomer, a pharmaceutically acceptable salt, ester, or prodrug thereof, comprises a compound of the formula:

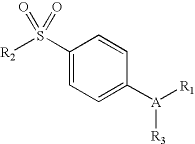

wherein

A is selected from the group consisting of partially unsaturated or unsaturated heterocyclyl and partially unsaturated or unsaturated carbocyclic rings;

R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R² is selected from the group consisting of methyl or amino; and

R³ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl.

In another embodiment the calcium modulating agent inhibits the intracellular passage of calcium ions through a voltage gated membrane channel. In one alternative of this embodiment, the voltage gated membrane channel is a high-voltage activated channel. In another alternative of this embodiment, the voltage gated membrane channel is a low-voltage activated channel.

In still another embodiment, the calcium modulating agent inhibits the intracellular passage of calcium ions through a receptor operated membrane channel.

In yet another embodiment, the calcium modulating agent is a calcium chelating agent.

Other aspects of the invention are described in more detail below.

Abbreviations and Definitions

The term “acyl” is a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl, and trifluoroacetyl.

The term “alkenyl” is a linear or branched radical having at least one carbon-carbon double bond of two to about twenty carbon atoms or, preferably, two to about twelve carbon atoms. More preferred alkyl radicals are “lower alkenyl” radicals having two to about six carbon atoms. Examples of alkenyl radicals include ethenyl, propenyl, allyl, propenyl, butenyl and 4-methylbutenyl.

The terms “alkenyl” and “lower alkenyl” also are radicals having “cis” and “trans” orientations, or alternatively, “E” and “Z” orientations. The term “cycloalkyl” is a saturated carbocyclic radical having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl.

The terms “alkoxy” and “alkyloxy” are linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy.

The term “alkoxyalkyl” is an alkyl radical having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The term “alkoxycarbonyl” is a radical containing an alkoxy radical, as defined above, attached via an oxygen atom to a carbonyl radical. More preferred are “lower alkoxycarbonyl” radicals with alkyl porions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl.

Where used, either alone or within other terms such as “haloalkyl”, “alkylsulfonyl”, “alkoxyalkyl” and “hydroxyalkyl”, the term “alkyl” is a linear, cyclic or branched radical having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “alkylamino” is an amino group that has been substituted with one or two alkyl radicals. Preferred are “lower N-alkylamino” radicals having alkyl portions having 1 to 6 carbon atoms. Suitable lower alkylamino may be mono or dialkylamino such as N-methylamino, N-ethylamino, N,N-dimethylamino, N,N-diethylamino or the like.

The term “alkylaminoalkyl” is a radical having one or more alkyl radicals attached to an aminoalkyl radical.

The term “alkylaminocarbonyl” is an aminocarbonyl group that has been substituted with one or two alkyl radicals on the amino nitrogen atom. Preferred are “N-alkylaminocarbonyl” “N,N-dialkylaminocarbonyl” radicals. More preferred are “lower N-alkylaminocarbonyl” “lower N,N-dialkylaminocarbonyl” radicals with lower alkyl portions as defined above.

The terms “alkylcarbonyl”, “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl.

The term “alkylthio” is a radical containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio.

The term “alkylthioalkyl” is a radical containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl.

The term “alkylsulfinyl” is a radical containing a linear or branched alkyl radical, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl.

The term “alkynyl” is a linear or branched radical having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The term “aminoalkyl” is an alkyl radical substituted with one or more amino radicals. More preferred are “lower aminoalkyl” radicals. Examples of such radicals include aminomethyl, aminoethyl, and the like.

The term “aminocarbonyl” is an amide group of the formula —C(═O)NH₂.

The term “aralkoxy” is an aralkyl radical attached through an oxygen atom to other radicals.

The term “aralkoxyalkyl” is an aralkoxy radical attached through an oxygen atom to an alkyl radical.

The term “aralkyl” is an aryl-substituted alkyl radical such as benzyl, diphenylmethyl, triphenylmethyl, phenylethyl, and diphenylethyl. The aryl in said aralkyl may be additionally substituted with halo, alkyl, alkoxy, halkoalkyl and haloalkoxy. The terms benzyl and phenylmethyl are interchangeable.

The term “aralkylamino” is an aralkyl radical attached through an amino nitrogen atom to other radicals. The terms “N-arylaminoalkyl” and “N-aryl-N-alkyl-aminoalkyl” are amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl.

The term “aralkylthio” is an aralkyl radical attached to a sulfur atom.

The term “aralkylthioalkyl” is an aralkylthio radical attached through a sulfur atom to an alkyl radical.

The term “aroyl” is an aryl radical with a carbonyl radical as defined above. Examples of aroyl include benzoyl, naphthoyl, and the like and the aryl in said aroyl may be additionally substituted.

The term “aryl”, alone or in combination, is a carbocyclic aromatic system containing one, two or three rings wherein such rings may be attached together in a pendent manner or may be fused. The term “aryl” includes aromatic radicals such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The term “arylamino” is an amino group, which has been substituted with one or two aryl radicals, such as N-phenylamino. The “arylamino” radicals may be further substituted on the aryl ring portion of the radical.

The term “aryloxyalkyl” is a radical having an aryl radical attached to an alkyl radical through a divalent oxygen atom.

The term “arylthioalkyl” is a radical having an aryl radical attached to an alkyl radical through a divalent sulfur atom.

The term “carbonyl”, whether used alone or with other terms, such as “alkoxycarbonyl”, is —(C═O)—.

The terms “carboxy” or “carboxyl”, whether used alone or with other terms, such as “carboxyalkyl”, is —CO2H.

The term “carboxyalkyl” is an alkyl radical substituted with a carboxy radical. More preferred are “lower carboxyalkyl” which are lower alkyl radicals as defined above, and may be additionally substituted on the alkyl radical with halo. Examples of such lower carboxyalkyl radicals include carboxymethyl, carboxyethyl and carboxypropyl.

The term “cycloalkenyl” is a partially unsaturated carbocyclic radical having three to twelve carbon atoms. More preferred cycloalkenyl radicals are “lower cycloalkenyl” radicals having four to about eight carbon atoms. Examples of such radicals include cyclobutenyl, cyclopentenyl, cyclopentadienyl, and cyclohexenyl.

The term “cyclooxygenase-2 selective inhibitor” is a compound able to inhibit cyclooxygenase-2 without significant inhibition of cyclooxygenase-1. Typically, it includes compounds that have a cyclooxygenase-2 IC₅₀ of less than about 0.2 micro molar, and also have a selectivity ratio of cyclooxygenase-2 inhibition over cyclooxygenase-1 inhibition of at least 50, and more typically, of at least 100. Even more typically, the compounds have a cyclooxygenase-1 IC₅₀ of greater than about 1 micro molar, and more preferably of greater than 10 micro molar. Inhibitors of the cyclooxygenase pathway in the metabolism of arachidonic acid used in the present method may inhibit enzyme activity through a variety of mechanisms. By the way of example, and without limitation, the inhibitors used in the methods described herein may block the enzyme activity directly by acting as a substrate for the enzyme.

The term “halo” is a halogen such as fluorine, chlorine, bromine or iodine.

The term “haloalkyl” is a radical wherein any one or more of the alkyl carbon atoms is substituted with halo as defined above. Specifically included are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” is a radical having 1-6 carbon atoms. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “heteroaryl” is an unsaturated heterocyclyl radical. Examples of unsaturated heterocyclyl radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like. The term also includes radicals where heterocyclyl radicals are fused with aryl radicals. Examples of such fused bicyclic radicals include benzofuran, benzothiophene, and the like. Said “heterocyclyl group” may have 1 to 3 substituents such as alkyl, hydroxyl, halo, alkoxy, oxo, amino and alkylamino.

The term “heterocyclyl” is a saturated, partially unsaturated and unsaturated heteroatom-containing ring-shaped radical, where the heteroatoms may be selected from nitrogen, sulfur and oxygen. Examples of saturated heterocyclyl radicals include saturated 3 to 6-membered heteromonocylic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole.

The term “heterocyclylalkyl” is a saturated and partially unsaturated heterocyclyl-substituted alkyl radical, such as pyrrolidinylmethyl, and heteroaryl-substituted alkyl radicals, such as pyridylmethyl, quinolylmethyl, thienylmethyl, furylethyl, and quinolylethyl. The heteroaryl in said heteroaralkyl may be additionally substituted with halo, alkyl, alkoxy, halkoalkyl and haloalkoxy.

The term “hydrido” is a single hydrogen atom (H). This hydrido radical may be attached, for example, to an oxygen atom to form a hydroxyl radical or two hydrido radicals may be attached to a carbon atom to form a methylene (—CH2-) radical.

The term “hydroxyalkyl” is a linear or branched alkyl radical having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The term “modulate,” as used herein, refers to a change in the biological activity of a biologically active molecule. Modulation can be an increase or a decrease in activity, a change in binding characteristics, or any other change in the biological, functional, or immunological properties of biologically active molecules.

The term “pharmaceutically acceptable” is used adjectivally herein to mean that the modified noun is appropriate for use in a pharmaceutical product; that is the “pharmaceutically acceptable” material is relatively safe and/or non-toxic, though not necessarily providing a separable therapeutic benefit by itself. Pharmaceutically acceptable cations include metallic ions and organic ions. More preferred metallic ions include, but are not limited to appropriate alkali metal salts, alkaline earth metal salts and other physiologically acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzy1ethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid, oxalacetic acid, fumaric acid, propionic acid, aspartic acid, glutamic acid, benzoic acid, and the like.

The term “prodrug” refers to a chemical compound that can be converted into a therapeutic compound by metabolic or simple chemical processes within the body of the subject. For example, a class of prodrugs of COX-2 inhibitors is described in U.S. Pat. No. 5,932,598, herein incorporated by reference.

The term “subject” for purposes of treatment includes any human or animal subject who is in need of such treatment. The subject can be a domestic livestock species, a laboratory animal species, a zoo animal or a companion animal. In one embodiment, the subject is a mammal. In another embodiment, the mammal is a human being.

The term “sulfonyl”, whether used alone or linked to other terms such as alkylsulfonyl, is a divalent radical —SO₂—. “Alkylsulfonyl” is an alkyl radical attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals. The terms “sulfamyl”, “aminosulfonyl” and “sulfonamidyl” are NH₂O₂S—.

The phrase “therapeutically-effective” is intended to qualify the amount of each agent (i.e. the amount of cyclooxygenase-2 selective inhibitor and the amount of calcium modulating agent) which will achieve the goal of improvement in disorder severity and the frequency of incidence over no treatment or treatment of each agent by itself.

### DESCRIPTION OF THE PREFERRED EMBODIMENTS

The present invention provides a combination therapy comprising the administration to a subject of a therapeutically effective amount of a COX-2 selective inhibitor in combination with a therapeutically effective amount of a calcium modulating agent. The combination therapy may be used to treat a pain, inflammation or an inflammation mediated disorder. When administered as part of a combination therapy, the COX-2 selective inhibitor together with the calcium modulating agent provide enhanced treatment options as compared to administration of either the calcium modulating agent or the COX-2 selective inhibitor alone.

Cyclooxygenase-2 Selective Inhibitors

A number of suitable cyclooxygenase-2 selective inhibitors or an isomer, a pharmaceutically acceptable salt, ester, or prodrug thereof, may be employed in the composition of the current invention. In one embodiment, the cyclooxygenase-2 selective inhibitor can be, for example, the cyclooxygenase-2 selective inhibitor meloxicam, Formula B-1 (CAS registry number 71125-38-7) or an isomer, a pharmaceutically acceptable salt, ester, or prodrug of a compound having Formula B-1.

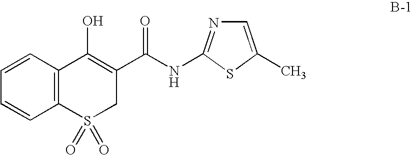

In yet another embodiment, the cyclooxygenase-2 selective inhibitor is the cyclooxygenase-2 selective inhibitor, 6-[[5-(4-chlorobenzoyl)-1,4-dimethyl-1H-pyrrol-2-yl]methyl]-3(2H)-pyridazinone, Formula B-2 (CAS registry number 179382-91-3) or an isomer, a pharmaceutically acceptable salt, ester, or prodrug of a compound having Formula B-2.

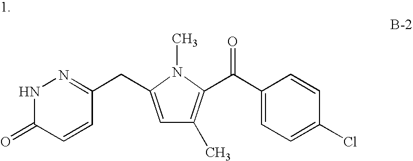

In still another embodiment the cyclooxygenase-2 selective inhibitor is a chromene compound that is a substituted benzopyran or a substituted benzopyran analog, and even more typically, selected from the group consisting of substituted benzothiopyrans, dihydroquinolines, dihydronaphthalenes or a compound having Formula I shown below and possessing, by way of example and not limitation, the structures disclosed in Table 1x. Furthermore, benzopyran cyclooxygenase-2 selective inhibitors useful in the practice of the present methods are described in U.S. Pat. Nos. 6,034,256 and 6,077,850 herein incorporated by reference in their entirety.

In another embodiment, the cyclooxygenase-2 selective inhibitor is a chromene compound represented by Formula I or an isomer, a pharmaceutically acceptable salt, ester, or prodrug thereof:

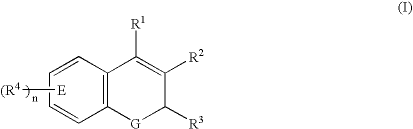

wherein:

n is an integer which is 0, 1, 2, 3 or4;

G is O, S or NR^{a};

R^{a} is alkyl

R¹ is selected from the group consisting of H and aryl;

R² is selected from the group consisting of carboxyl, aminocarbonyl, alkylsulfonylaminocarbonyl and alkoxycarbonyl;

R³ is selected from the group consisting of haloalkyl, alkyl, aralkyl, cycloalkyl and aryl optionally substituted with one or more radicals selected from alkylthio, nitro and alkylsulfonyl; and

each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;

or R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof, wherein:

n is an integer which is 0, 1, 2, 3 or 4;

G is O, S or NR^{a};

R¹ is H;

R^{a} is alkyl;

R² is selected from the group consisting of carboxyl, aminocarbonyl, alkylsulfonylaminocarbonyl and alkoxycarbonyl;

R³ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and

each R⁴ is independently selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁴ together with ring E forms a naphthyl radical.

In a further embodiment, the cyclooxygenase-2 selective inhibitor may also be a compound of Formula (I), or an isomer, a pharmaceutically acceptable salt, ester, or prodrug thereof, wherein:

n is an integer which is 0, 1, 2, 3 or 4;

G is oxygen or sulfur;

R¹ is H;

R² is carboxyl, lower alkyl, lower aralkyl or lower alkoxycarbonyl;

R³ is lower haloalkyl, lower cycloalkyl or phenyl; and

each R⁴ is H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, or lower alkylcarbonyl; or

R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof wherein:

R²is carboxyl;

R³ is lower haloalkyl; and

each R⁴ is H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl; or wherein R⁴ together with ring E forms a naphthyl radical.

The cyclooxygenase-2 selective inhibitor may also be a compound of Formula (I) or an isomer, a pharmaceutically acceptable salt, ester, or prodrug thereof wherein:

n is an integer which is 0, 1, 2, 3 or 4;

R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and

each R⁴ is H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl or phenyl; or wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof wherein:

n is an integer which is 0, 1, 2, 3 or4;

R³ is trifluoromethyl or pentafluoroethyl; and

each R⁴ is independently H, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, or phenyl; or wherein R⁴ together with the carbon atoms to which it is attached and the remainder of ring E forms a naphthyl radical.

In yet another embodiment, the cyclooxygenase-2 selective inhibitor used in connection with the method(s) of the present invention can also be a compound having the structure of Formula (I) or an isomer, a pharmaceutically acceptable salt, ester, or prodrug thereof wherein:

n=4;

G is O or S;

R¹ is H;

R²is CO₂H;

R³ is lower haloalkyl;

a first R⁴ corresponding to R⁹ is hydrido or halo;

a second R⁴ corresponding to R¹⁰ is H, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, or 6-membered nitrogen-containing heterocyclosulfonyl;

a third R⁴ corresponding to R¹¹ is H, lower alkyl, halo, lower alkoxy, or aryl; and

a fourth R⁴ corresponding to R¹² is H, halo, lower alkyl, lower alkoxy, and aryl;

wherein Formula (I) is represented by Formula (Ia):

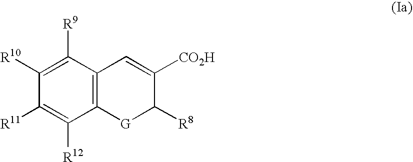

The cyclooxygenase-2 selective inhibitor used in connection with the method(s) of the present invention can also be a compound of having the structure of Formula (Ia) or an isomer, a pharmaceutically acceptable salt, ester, or prodrug thereof wherein:

R⁸ is trifluoromethyl or pentafluoroethyl;

R⁹ is H, chloro, or fluoro;

R¹⁰ is H, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, or morpholinosulfonyl;

R¹¹ is H, methyl, ethyl, isopropyl, tert-butyl, chloro, methoxy, diethylamino, or phenyl; and

R¹² is H, chloro, bromo, fluoro, methyl, ethyl, tert-butyl, methoxy, or phenyl.

Examples of exemplary chromene cyclooxygenase-2 selective inhibitors are depicted in Table 1x below.

TABLE 1X
EXAMPLES OF CHROMENE CYCLOOXYGENASE-2 SELECTIVE
INHIBITORS AS EMBODIMENTS

| Compound | |
|---|---|
| Number | Structural Formula |
| B-3 | 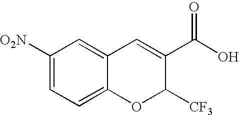 |
| | 6-Nitro-2-trifluorornethyl-2H-1- |
| | benzopyran-3-carboxylic acid |
| B-4 | 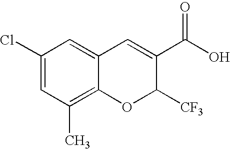 |
| | 6-Chloro-8-methyl-2-trifluoromethyl- |
| | 2H-1-benzopyran-3-carboxylic acid |
| B-5 | 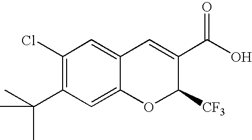 |
| | ((S)-6-Chloro-7-(1,1-dimethylethyl)-2-(trifluoro- |
| | methyl-2H-1-benzopyran-3-carboxylic acid |
| B-6 | 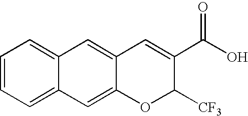 |
| | 2-Trifluoromethyl-2H-naphtho[2,3-b] |
| | pyran-3-carboxylic acid |
| B-7 | 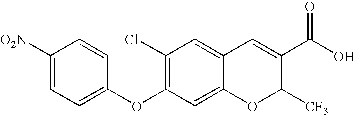 |
| | 6-Chloro-7-(4-nitrophenoxy)-2-(trifluoromethyl)-2H-1- |
| | benzopyran-3-carboxylic acid |
| B-8 | 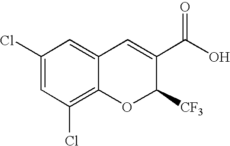 |
| | ((S)-6,8-Dichloro-2-(trifluoromethyl)- |
| | 2H-1-benzopyran-3-carboxylic acid |
| B-9 | 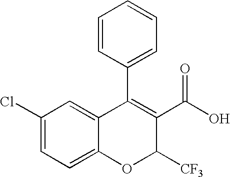 |
| | 6-Chloro-2-(trifluoromethyl)-4-phenyl-2H- |
| | 1-benzopyran-3-carboxylic acid |
| B-10 | 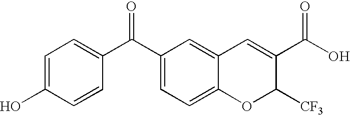 |
| | 6-(4-Hydroxybenzoyl)-2-(trifluoromethyl)- |
| | 2H-1-benzopyran-3-carboxylic acid |
| B-11 | 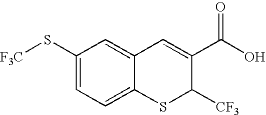 |
| | 2-(Trifluoromethyl)-6-[(trifluoromethyl)thio]- |
| | 2H-1-benzothiopyran-3-carboxylic acid |
| B-12 | 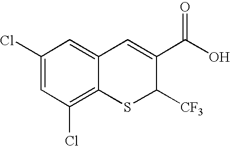 |
| | 6,8-Dichloro-2-trifluoromethyl-2H-1- |
| | benzothiopyran-3-carboxylic acid |
| B-13 | 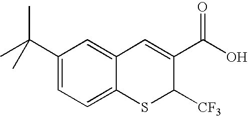 |
| | 6-(1,1-Dimethylethyl)-2-(trifluoromethyl)- |
| | 2H-1-benzothiopyran-3-carboxylic acid |
| B-14 | 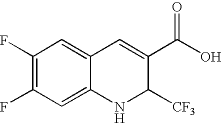 |
| | 6,7-Difluoro-1,2-dihydro-2-(trifluoro- |
| | methyl)-3-quinolinecarboxylic acid |
| B-15 | 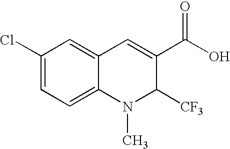 |
| | 6-Chloro-1,2-dihydro-1-methyl-2-(trifluoro- |
| | methyl)-3-quinolinecarboxylic acid |
| B-16 | 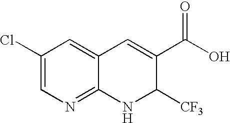 |
| | 6-Chloro-2-(trifluoromethyl)-1,2-dihydro |
| | [1,8]naphthyridine-3-carboxylic acid |
| B-17 | 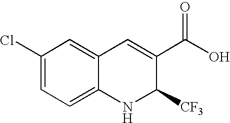 |
| | ((S)-6-Chloro-1,2-dihydro-2-(trifluoro- |
| | methyl)-3-quinolinecarboxylic acid |

In a further embodiment, the cyclooxygenase-2 selective inhibitor is selected from the class of tricyclic cyclooxygenase-2 selective inhibitors represented by the general structure of Formula I: or an isomer, a pharmaceutically acceptable salt, ester, or prodrug thereof wherein:

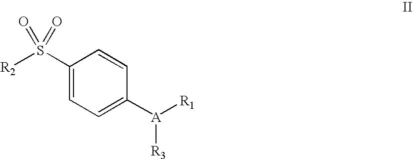

A is selected from the group consisting of partially unsaturated or unsaturated heterocyclyl and partially unsaturated or unsaturated carbocyclic rings;

R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R² is selected from the group consisting of methyl or amino; and

R³ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl.

In another embodiment, the cyclooxygenase-2 selective inhibitor represented by the above Formula II is selected from the group of compounds illustrated in Table 2x, consisting of celecoxib (B-18; U.S. Pat. No. 5,466,823; CAS No. 169590-42-5), valdecoxib (B-19; U.S. Pat. No. 5,633,272; CAS No.181695-72-7), deracoxib (B-20; U.S. Pat. No. 5,521,207; CAS No. 169590-41-4), rofecoxib (B-21; CAS No.162011-90-7), etoricoxib (MK-663; B-22; PCT publication WO 98/03484), tilmacoxib (JTE-522; B-23; CAS No.180200-68-4).

TABLE 2X
EXAMPLES OF TRICYCLIC CYCLOOXYGENASE-2 SELECTIVE
INHIBITORS AS EMBODIMENTS

| Compound | |
|---|---|
| Number | Structural Formula |
| B-18 | 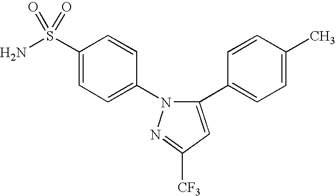 |
| B-19 | 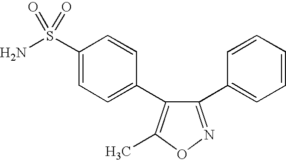 |
| B-20 | 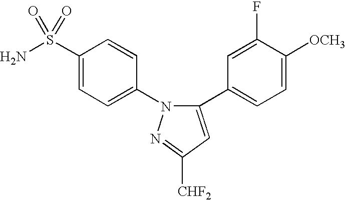 |
| B-21 | 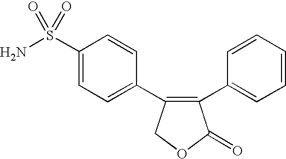 |
| B-22 | 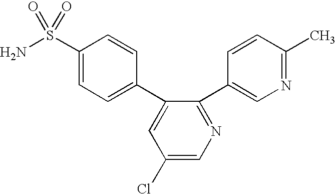 |
| B-23 | 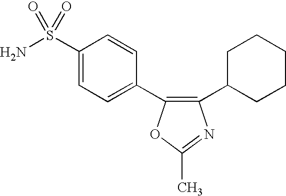 |

In still another embodiment, the cyclooxygenase-2 selective inhibitor is selected from the group consisting of celecoxib, rofecoxib and etoricoxib.

In yet another embodiment, the cyclooxygenase-2 selective inhibitor is parecoxib (B-24, U.S. Pat. No. 5,932,598, CAS No.198470-84-7), which is a therapeutically effective prodrug of the tricyclic cyclooxygenase-2 selective inhibitor valdecoxib, B-19, may be advantageously employed as a source of a cyclooxygenase inhibitor (U.S. Pat. No. 5,932,598, herein incorporated by reference).

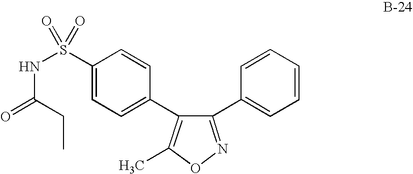

One form of parecoxib is sodium parecoxib.

In another embodiment of the invention, the compound having the formula B-25 or an isomer, a pharmaceutically acceptable salt, ester, or prodrug of a compound having formula B-25 that has been previously described in International Publication number WO 00/24719 (which is herein incorporated by reference) is another tricyclic cyclooxygenase-2 selective inhibitor that may be advantageously employed.

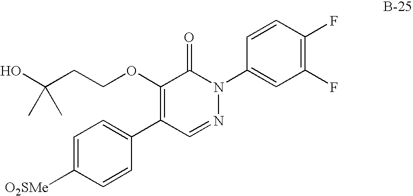

Another cyclooxygenase-2 selective inhibitor that is useful in connection with the method(s) of the present invention is N-(2-cyclohexyloxynitrophenyl)-methane sulfonamide (NS-398) having a structure shown below as B-26, or an isomer, a pharmaceutically acceptable salt, ester, or prodrug of a compound having formula B-26.

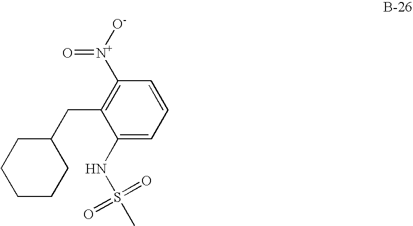

In yet a further embodiment, the cyclooxygenase-2 selective inhibitor used in connection with the method(s) of the present invention can be selected from the class of phenylacetic acid derivative cyclooxygenase-2 selective inhibitors represented by the general structure of Formula (III) or an isomer, a pharmaceutically acceptable salt, ester, or prodrug thereof:

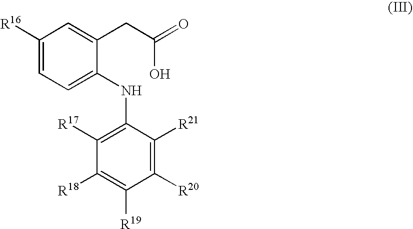

wherein:

R¹⁶ is methyl or ethyl;

R¹⁷ is chloro or fluoro;

R¹⁸ is hydrogen or fluoro;

R¹⁹ is hydrogen, fluoro, chloro, methyl, ethyl, methoxy, ethoxy or hydroxy;

R²⁰ is hydrogen or fluoro; and

R²¹ is chloro, fluoro, trifluoromethyl or methyl, provided that R¹⁷, R¹⁸, R¹⁹ and R²⁰ are not all fluoro when R¹⁶ is ethyl and R¹⁹ is H.

Another phenylacetic acid derivative cyclooxygenase-2 selective inhibitor used in connection with the method(s) of the present invention is a compound that has the designation of COX 189 (lumiracoxib; B-211) and that has the structure shown in Formula (III) or an isomer, a pharmaceutically acceptable salt, ester, or prodrug thereof wherein:

R¹⁶ is ethyl;

R¹⁷ and R¹⁹ are chloro;

R¹⁸ and R²⁰ are hydrogen; and

and R²¹ is methyl.

In yet another embodiment, the cyclooxygenase-2 selective inhibitor is represented by Formula (IV) or an isomer, a pharmaceutically acceptable salt, ester, or prodrug thereof:

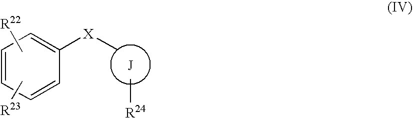

wherein:

X is O or S;

J is a carbocycle or a heterocycle;

R²² is NHSO₂CH₃ or F;

R²³ is H, NO₂, or F; and

R²⁴ is H, NHSO₂CH₃, or (SO₂CH₃)C₆H₄.

According to another embodiment, the cyclooxygenase-2 selective inhibitors used in the present method(s) have the structural Formula (V) or an isomer, a pharmaceutically acceptable salt, ester, or prodrug thereof:

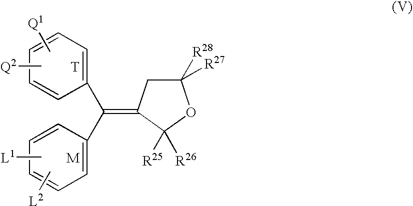

wherein:

T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;

Q¹, Q², L¹ or L² are independently hydrogen, halogen, lower alkyl having from 1 to 6 carbon atoms, trifluoromethyl, or lower methoxy having from 1 to 6 carbon atoms; and

at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,

Q¹ and Q² are methylenedioxy; or

L¹ and L² are methylenedioxy; and

R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,

R²⁵ and R²⁶ are O; or,

R²⁷ and R²⁸ are O; or,

R²⁵, R²⁶, together with the carbon atom to which they are attached, form a saturated hydrocarbon ring having from 3 to 7 carbon atoms; or,

R²⁷, R²⁸, together with the carbon atom to which they are attached, form a saturated hydrocarbon ring having from 3 to 7 carbon atoms.

In another embodiment, the compounds N-(2-cyclohexyloxynitrophenyl)methane sulfonamide, and (E)-4-[(4-methylphenyl)(tetrahydro-2-oxo-3-furanylidene) methyl]benzenesulfonamide or an isomer, a pharmaceutically acceptable salt, ester, or prodrug thereof having the structure of Formula (V) are employed as cyclooxygenase-2 selective inhibitors.

In a further embodiment, compounds that are useful for the cyclooxygenase-2 selective inhibitor or an isomer, a pharmaceutically acceptable salt, ester, or prodrug thereof used in connection with the method(s) of the present invention, the structures for which are set forth in Table 3x below, include, but are not limited to:

6-chloro-2-trifluoromethyl-2H-1-benzopyran-3-carboxylic acid (B-27);

6-chloro-7-methyl-2-trifluoromethyl-2H-1-benzopyran-3-carboxylic acid (B-28);

8-(1-methylethyl)-2-trifluoromethyl-2H-1-benzopyran-3-carboxylic acid (B-29);

6-chloro-8-(1-methylethyl)-2-trifluoromethyl-2H-1 -benzopyran-3-carboxylic acid (B-30);

2-trifluoromethyl-3H-naphtho[2,1-b]pyran-3-carboxylic acid (B-31);

7-(1,1-dimethylethyl)-2-trifluoromethyl-2H-1-benzopyran-3-carboxylic acid (B-32);

6-bromo-2-trifluoromethyl-2H-1-benzopyran-3-carboxylic acid (B-33);

8-chloro-2-trifluoromethyl-2H-1-benzopyran-3-carboxylic acid (B-34);

6-trifluoromethoxy-2-trifluoromethyl-2H-1-benzopyran-3-carboxylic acid (B-35);

5,7-dichloro-2-trifluoromethyl-2H-1-benzopyran-3-carboxylic acid (B-36);

8-phenyl-2-trifluoromethyl-2H-1-benzopyran-3-carboxylic acid (B-37);

7,8-dimethyl-2-trifluoromethyl-2H-1-benzopyran-3-carboxylic acid (B-38);

6,8-bis(dimethylethyl)-2-trifluoromethyl-2H-1-benzopyran-3-carboxylic acid (B-39);

7-(1-methylethyl)-2-trifluoromethyl-2H-1-benzopyran-3-carboxylic acid (B-40);

7-phenyl-2-trifluoromethyl-2H-1-benzopyran-3-carboxylic acid (B-41);

6-chloro-7-ethyl-2-trifluoromethyl-2H-1-benzopyran-3-carboxylic acid (B-42);

6-chloro-8-ethyl-2-trifluoromethyl-2H-1-benzopyran-3-carboxylic acid (B-43);

6-chloro-7-phenyl-2-trifluoromethyl-2H-1-benzopyran-3-carboxylic acid (B44);

6,7-dichloro-2-trifluoromethyl-2H-1-benzopyran-3-carboxylic acid (B-45);

6,8-dichloro-2-trifluoromethyl-2H-1-benzopyran-3-carboxylic acid (B-46);

6-chloro-8-methyl-2-trifluoromethyl-2H-1-benzopyran-3-carboxylic acid (B-47);

8-chloro-6-methyl-2-trifluoromethyl-2H-1-benzopyran-3-carboxylic acid (B-48)

8-chloro-6-methoxy-2-trifluoromethyl-2H-1-benzopyran-3-carboxylic acid (B-49);

6-bromo-8-chloro-2-trifluoromethyl-2H-1-benzopyran-3-carboxylic acid (B-50);

8-bromo-6-fluoro-2-trifluoromethyl-2H-1-benzopyran-3-carboxylic acid (B-51);

8-bromo-6-methyl-2-trifluoromethyl-2H-1-benzopyran-3-carboxylic acid (B-52);

8-bromo-5-fluoro-2-trifluoromethyl-2H-1-benzopyran-3-carboxylic acid (B-53);

6-chloro-8-fluoro-2-trifluoromethyl-2H-1-benzopyran-3-carboxylic acid (B-54);

6-bromo-8-methoxy-2-trifluoromethyl-2H-1-benzopyran-3-carboxylic acid (B-55);

6-[[(phenylmethyl)amino]sulfonyl]-2-trifluoromethyl-2H-1-benzopyran-3-carboxylic acid (B-56);

6-[(dimethylamino)sulfonyl]-2-trifluoromethyl-2H-1-benzopyran-3-carboxylic acid (B-57);

6-[(methylamino)sulfonyl]-2-trifluoromethyl-2H-1-benzopyran-3-carboxylic acid (B-58);

6-[(4-morpholino)sulfonyl]-2-trifluoromethyl-2H-1-benzopyran-3-carboxylic acid (B-59);

6-[(1,1-dimethylethyl)aminosulfonyl]-2-trifluoromethyl-2H-1-benzopyran-3-carboxylic acid (B-60);

6-[(2-methylpropyl)aminosulfonyl]-2-trifluoromethyl-2H-1-benzopyran-3-carboxylic acid (B-61);

6-methylsulfonyl-2-trifluoromethyl-2H-1-benzopyran-3-carboxylic acid (B-62);

8-chloro-6-[[(phenylmethyl)amino]sulfonyl]-2-trifluoromethyl-2H-1-benzopyran-3-carboxylic acid (B-63);

6-phenylacetyl-2-trifluoromethyl-2H-1-benzopyran-3-carboxylic acid (B-64);

6,8-dibromo-2-trifluoromethyl-2H-1-benzopyran-3-carboxylic acid (B-65);

8-chloro-5,6-dimethyl-2-trifluoromethyl-2H-1-benzopyran-3-carboxylic acid (B-66);

6,8-dichloro-(S)-2-trifluoromethyl-2H-1-benzopyran-3-carboxylic acid (B-67);

6-benzylsulfonyl-2-trifluoromethyl-2H-1-benzopyran-3-carboxylic acid (B-68);

6-[[N-(2-furylmethyl)amino]sulfonyl]-2-trifluoromethyl-2H-1-benzopyran-3-carboxylic acid (B-69);

6-[[N-(2-phenylethyl)amino]sulfonyl]-2-trifluoromethyl-2H-1-benzopyran-3-carboxylic acid (B-70);

6-iodo-2-trifluoromethyl-2H-1-benzopyran-3-carboxylic acid (B-71);

7-(1,1-dimethylethyl)-2-pentafluoroethyl-2H-1-benzopyran-3-carboxylic acid (B-72);

6-chloro-2-trifluoromethyl-2H-1-benzothiopyran-3-carboxylic acid (B-73);

3-[(3-Chloro-phenyl)-(4-methanesulfonyl-phenyl)-methylene]-dihydro-furan-2-one or BMS-347070 (B-74);

8-acetyl-3-(4-fluorophenyl)-2-(4-methylsulfonyl)phenyl-imidazo(1,2-a)pyridine (B-75);

5,5-dimethyl-4-(4-methylsulfonyl)phenyl-3-phenyl-2-(5H )-furanone (B-76);

5-(4-fluorophenyl)-1-[4-(methylsulfonyl)phenyl]-3-(trifluoromethyl)pyrazole (B-77);

4-(4-fluorophenyl)-5-[4-(methylsulfonyl)phenyl]-1-phenyl-3-(trifluoromethyl)pyrazole (B-78);

4-(5-(4-chlorophenyl)-3-(4-methoxyphenyl)-1H-pyrazol-1-yl)benzenesulfonamide (B-79);

4-(3,5-bis(4-methylphenyl)-1H-pyrazol-1-yl)benzenesulfonamide (B-80);

4-(5-(4-chlorophenyl)-3-phenyl-1H-pyrazol-1-yl)benzenesulfonamide (B-81);

4-(3,5-bis(4-methoxyphenyl)-1H-pyrazol-1-yl)benzenesulfonamide (B-82);

4-(5-(4-chlorophenyl)-3-(4-methylphenyl)-1H-pyrazol-1-yl)benzenesulfonamide (B-83);

4-(5-(4-chlorophenyl)-3-(4-nitrophenyl)-1H-pyrazol-1-yl)benzenesulfonamide (B-84);

4-(5-(4-chlorophenyl)-3-(5-chloro-2-thienyl)-1H-pyrazol-1-yl)benzenesulfonamide (B-85);

4-(4-chloro-3,5-diphenyl-1H-pyrazol-1-yl)benzenesulfonamide (B-86);

4-[5-(4-chlorophenyl)-3-(trifluoromethyl)-1H-pyrazol-1-yl]benzenesulfonamide (B-87);

4-[5-phenyl-3-(trifluoromethyl)-1H-pyrazol-1-yl]benzenesulfonamide (B-88);

4-[5-(4-fluorophenyl)-3-(trifluoromethyl)-1H-pyrazol-1-yl]benzenesulfonamide (B-89);

4-[5-(4-methoxyphenyl)-3-(trifluoromethyl)-1H-pyrazol-1-yl]benzenesulfonamide (B-90);

4-[5-(4-chlorophenyl)-3-(difluoromethyl)-1H-pyrazol-1-yl]benzenesulfonamide (B-91);

4-[5-(4-methylphenyl)-3-(trifluoromethyl)-1H-pyrazol-1-yl]benzenesulfonamide (B-92);

4-[4-chloro-5-(4-chlorophenyl)-3-(trifluoromethyl)-1H-pyrazol-1-yl]benzenesulfonamide (B-93);

4-[3-(difluoromethyl)-5-(4-methylphenyl)-1H-pyrazol-1-yl]benzenesulfonamide (B-94);

4-[3-(difluoromethyl)-5-phenyl-1H-pyrazol-1-yl]benzenesulfonamide (B-95);

4-[3-(difluoromethyl)-5-(4-methoxyphenyl)-1H-pyrazol-1-yl]benzenesulfonamide (B-96);

4-[3-cyano-5-(4-fluorophenyl)-1H-pyrazol-1-yl]benzenesulfonamide (B-97);

4-[3-(difluoromethyl)-5-(3-fluoro-4-methoxyphenyl)-1H-pyrazol-1-yl]benzenesulfonamide (B-98);

4-[5-(3-fluoro-4-methoxyphenyl)-3-(trifluoromethyl)-1H-pyrazol-1-yl]benzenesulfonamide (B-99);

4-[4-chloro-5-phenyl-1H-pyrazol-1-yl]benzenesulfonamide (B-100);

4-[5-(4-chlorophenyl)-3-(hydroxymethyl)-1H-pyrazol-1-yl]benzenesulfonamide (B-101);

4-[5-(4-(N,N-dimethylamino)phenyl)-3-(trifluoromethyl)-1H-pyrazol-1-yl]benzenesulfonamide (B-102);

5-(4-fluorophenyl)-6-[4-(methylsulfonyl)phenyl]spiro[2.4]hept-5-ene (B-103);

4-[6-(4-fluorophenyl)spiro[2.4]hept-5-en-5-yl]benzenesulfonamide (B-104);

6-(4-fluorophenyl)-7-[4-(methylsulfonyl)phenyl]spiro[3.4]oct-6-ene (B-105);

5-(3-chloro-4-methoxyphenyl)-6-[4-(methylsulfonyl)phenyl]spiro[2.4]hept-5-ene (B-106);

4-[6-(3-chloro-4-methoxyphenyl)spiro[2.4]hept-5-en-5-yl]benzenesulfonamide (B-107);

5-(3,5-dichloro-4-methoxyphenyl)-6-[4-(methylsulfonyl)phenyl]spiro[2.4]hept-5-ene (B-108);

5-(3-chloro-4-fluorophenyl)-6-[4-(methylsulfonyl)phenyl]spiro[2.4]hept-5-ene (B-109);

4-[6-(3,4-dichlorophenyl)spiro[2.4]hept-5-en-5-yl]benzenesulfonamide (B-110);

2-(3-chloro-4-fluorophenyl)-4-(4-fluorophenyl)-5-(4-methylsulfonylphenyl)thiazole (B-111);

2-(2-chlorophenyl)-4-(4-fluorophenyl)-5-(4-methylsulfonylphenyl)thiazole (B-112);

5-(4-fluorophenyl)-4-(4-methylsulfonylphenyl)-2-methylthiazole (B-113);

4-(4-fluorophenyl)-5-(4-methylsulfonylphenyl)-2-trifluoromethylthiazole (B-114);

4-(4-fluorophenyl)-5-(4-methylsulfonylphenyl)-2-(2-thienyl)thiazole (B-115);

4-(4-fluorophenyl )-5-(4-methylsulfonylphenyl)-2-benzylaminothiazole (B-116);

4-(4-fluorophenyl)-5-(4-methylsulfonylphenyl)-2-(1-propylamino)thiazole (B-117);

2-[(3,5-dichlorophenoxy)methyl)-4-(4-fluorophenyl)-5-[4-(methylsulfonyl)phenyl]thiazole (B-118);

5-(4-fluorophenyl)-4-(4-methylsulfonylphenyl)-2-trifluoromethylthiazole (B-119);

1-methylsulfonyl-4-[1,1-dimethyl-4-(4-fluorophenyl)cyclopenta-2,4-dien-3-yl]benzene (B-120);

4-[4-(4-fluorophenyl)-1,1-dimethylcyclopenta-2,4-dien-3-yl]benzenesulfonamide (B-121);

5-(4-fluorophenyl)-6-[4-(methylsulfonyl)phenyl]spiro[2.4]hepta4,6-diene (B-122);

4-[6-(4-fluorophenyl)spiro[2.4]hepta4,6-dien-5-yl]benzenesulfonamide (B-123);

6-(4-fluorophenyl)-2-methoxy-5-[4-(methylsulfonyl)phenyl]-pyridine-3-carbonitrile (B-124);

2-bromo-6-(4-fluorophenyl)-5-[4-(methylsulfonyl)phenyl]-pyridine-3-carbonitrile (B-125);

6-(4-fluorophenyl)-5-[4-(methylsulfonyl)phenyl]-2-phenyl-pyridine-3-carbonitrile (B-126);

4-[2-(4-methylpyridin-2-yl)-4-(trifluoromethyl)-1H-imidazol-1-yl]benzenesulfonamide (B-127);

4-[2-(5-methylpyridin-3-yl)-4-(trifluoromethyl)-1H-imidazol-1-yl]benzenesulfonamide (B-128);

4-[2-(2-methylpyridin-3-yl)-4-(trifluoromethyl)-1H-imidazol-1-yl]benzenesulfonamide (B-129);

3-[1-[4-(methylsulfonyl)phenyl]-4-(trifluoromethyl)-1H-imidazol-2-yl]pyridine (B-130);

2-[1-[4-(methylsulfonyl)phenyl-4-(trifluoromethyl)-1H-imidazol-2-yl]pyridine (B-131);

2-methyl-4-[1-[4-(methylsulfonyl)phenyl-4-(trifluoromethyl)-1H-imidazol-2-yl]pyridine (B-132);

2-methyl-6-[1-[4-(methylsulfonyl)phenyl-4-(trifluoromethyl)-1H-imidazol-2-yl]pyridine (B-133);

4-[2-(6-methylpyridin-3-yl)-4-(trifluoromethyl)-1H-imidazol-1-yl]benzenesulfonamide (B-134);

2-(3,4-difluorophenyl)-1-[4-(methylsulfonyl)phenyl]-4-(trifluoromethyl)-1H-imidazole (B-135);

4-[2-(4-methylphenyl)-4-(trifluoromethyl)-1H-imidazol-1-yl]benzenesulfonamide (B-136);

2-(4-chlorophenyl)-1-[4-(methylsulfonyl)phenyl]-4-methyl-1H-imidazole (B-137);

2-(4-chlorophenyl)-1-[4-(methylsulfonyl)phenyl]-4-phenyl-1H-imidazole (B-138);

2-(4-chlorophenyl)-4-(4-fluorophenyl)-1-[4-(methylsulfonyl)phenyl]-1H-imidazole (B-139);

2-(3-fluoro-4-methoxyphenyl)-1-[4-(methylsulfonyl)phenyl-4-(trifluoromethyl)-1H-imidazole (B-140);

1-[4-(methylsulfonyl)phenyl]-2-phenyl-4-trifluoromethyl-1H-imidazole (B-141);

2-(4-methylphenyl)-1-[4-(methylsulfonyl)phenyl]-4-trifluoromethyl-1H-imidazole (B-142);

4-[2-(3-chloro-4-methylphenyl)-4-(trifluoromethyl)-l H-imidazol-1-yl]benzenesulfonamide (B-143);

2-(3-fluoro-5-methylphenyl)-1-[4-(methylsulfonyl)phenyl]-4-(trifluoromethyl)-1H-imidazole (B-144);

4-[2-(3-fluoro-5-methylphenyl)-4-(trifluoromethyl)-1H-imidazol-1-yl]benzenesulfonamide (B-145);

2-(3-methylphenyl)-1-[4-(methylsulfonyl)phenyl]-4-trifluoromethyl-1H-imidazole (B-146);

4-[2-(3-methylphenyl)-4-trifluoromethyl-1H-imidazol-1-yl]benzenesulfonamide (B-147);

1-[4-(methylsulfonyl)phenyl]-2-(3-chlorophenyl)-4-trifluoromethyl-1H-imidazole (B-148);

4-[2-(3-chlorophenyl)-4-trifluoromethyl-1H-imidazol-1-yl]benzenesulfonamide (B-149);

4-[2-phenyl-4-trifluoromethyl-1H-imidazol-1-yl]benzenesulfonamide (B-150);

4-[2-(4-methoxy-3-chlorophenyl)-4-trifluoromethyl-1H-imidazol-1-yl]benzenesulfonamide (B-151);

1-allyl-4-(4-fluorophenyl)-3-[4-(methylsulfonyl)phenyl]-5-(trifluoromethyl)-1H-pyrazole (B-152);

4-[1-ethyl-4-(4-fluorophenyl)-5-(trifluoromethyl)-1H-pyrazol-3-yl]benzenesulfonamide (B-153);

N-phenyl-[4-(4-fluorophenyl)-3-[4-(methylsulfonyl)phenyl]-5-(trifluoromethyl)-1H-pyrazol-1-yl]acetamide (B-154);

ethyl [4-(4-fluorophenyl)-3-[4-(methylsulfonyl)phenyl]-5-(trifluoromethyl)-1H-pyrazol-1-yl]acetate (B-155);

4-(4-fluorophenyl)-3-[4-(methylsulfonyl)phenyl]-1-(2-phenylethyl)-1H-pyrazole (B-156);

4-(4-fluorophenyl)-3-[4-(methylsulfonyl)phenyl]-1-(2-phenylethyl)-5-(trifluoromethyl)pyrazole (B-157);

1-ethyl-4-(4-fluorophenyl)-3-[4-(methylsulfonyl)phenyl]-5-(trifluoromethyl)-1H-pyrazole (B-158);

5-(4-fluorophenyl)-4-(4-methylsulfonylphenyl)-2-trifluoromethyl-1H-imidazole (B-159);

4-[4-(methylsulfonyl)phenyl]-5-(2-thiophenyl)-2-(trifluoromethyl)-1H-imidazole (B-160);

5-(4-fluorophenyl)-2-methoxy-4-[4-(methylsulfonyl)phenyl]-6-(trifluoromethyl)pyridine (B-161);

2-ethoxy-5-(4-fluorophenyl)-4-[4-(methylsulfonyl)phenyl]-6-(trifluoromethyl)pyridine (B-162);

5-(4-fluorophenyl)-4-[4-(methylsulfonyl)phenyl]-2-(2-propynyloxy)-6-(trifluoromethyl)pyridine (B-163);

2-bromo-5-(4-fluorophenyl)-4-[4-(methylsulfonyl)phenyl]-6-(trifluoromethyl)pyridine (B-164);

4-[2-(3-chloro-4-methoxyphenyl)4,5-difluorophenyl]benzenesulfonamide (B-165);

1-(4-fluorophenyl)-2-[4-(methylsulfonyl)phenyl]benzene (B-166);

5-difluoromethyl-4-(4-methylsulfonylphenyl)-3-phenylisoxazole (B-167);

4-[3-ethyl-5-phenylisoxazol-4-yl]benzenesulfonamide (B-168);

4-[5-difluoromethyl-3-phenylisoxazol-4-yl]benzenesulfonamide (B-169);

4-[5-hydroxymethyl-3-phenylisoxazol-4-yl]benzenesulfonamide (B-170);

4-[5-methyl-3-phenyl-isoxazol-4-yl]benzenesulfonamide (B-171);

1-[2-(4-fluorophenyl)cyclopenten-1-yl]-4-(methylsulfonyl)benzene (B-172);

1-[2-(4-fluoro-2-methylphenyl)cyclopenten-1-yl]-4-(methylsulfonyl)benzene (B-173);

1-[2-(4-chlorophenyl)cyclopenten-1-yl]-4-(methylsulfonyl)benzene (B-174);

1-[2-(2,4-dichlorophenyl)cyclopenten-1-yl]-4-(methylsulfonyl)benzene (B-175);

1-[2-(4-trifluoromethylphenyl)cyclopenten-1-yl]-4-(methylsulfonyl)benzene (B-176);

1-[2-(4-methylthiophenyl)cyclopenten-1-yl]-4-(methylsulfonyl)benzene (B-177);

1-[2-(4-fluorophenyl)-4,4-dimethylcyclopenten-1-yl]-4-(methylsulfonyl)benzene (B-178);

4-[2-(4-fluorophenyl)-4,4-dimethylcyclopenten-1-yl]benzenesulfonamide (B-179);

1-[2-(4-chlorophenyl)-4,4-dimethylcyclopenten-1-yl]-4-(methylsulfonyl)benzene (B-180);

4-[2-(4-chlorophenyl)-4,4-dimethylcyclopenten-1-yl]benzenesulfonamide (B-181);

4-[2-(4-fluorophenyl)cyclopenten-1-yl]benzenesulfonamide (B-182);

4-[2-(4-chlorophenyl)cyclopenten-1-yl]benzenesulfonamide (B-183);

1-[2-(4-methoxyphenyl)cyclopenten-1-yl]-4-(methylsulfonyl)benzene (B-184);

1-[2-(2,3-difluorophenyl)cyclopenten-1-yl]-4-(methylsulfonyl)benzene (B-185);

4-[2-(3-fluoro-4-methoxyphenyl)cyclopenten-1-yl]benzenesulfonamide (B-186);

1-[2-(3-chloro-4-methoxyphenyl)cyclopenten-1-yl]-4-(methylsulfonyl)benzene (B-187);

4-[2-(3-chloro-4-fluorophenyl)cyclopenten-1-yl]benzenesulfonamide (B-188);

4-[2-(2-methylpyridin-5-yl)cyclopenten-1-yl]benzenesulfonamide (B-189);

ethyl 2-[4-(4-fluorophenyl)-5-[4-(methylsulfonyl)phenyl]oxazol-2-yl]-2-benzyl-acetate (B-190);

2-[4-(4-fluorophenyl)-5-[4-(methylsulfonyl)phenyl]oxazol-2-yl]acetic acid (B-191);

2-(tert-butyl)-4-(4-fluorophenyl)-5-[4-(methylsulfonyl)phenyl]oxazole (B-192);

4-(4-fluorophenyl)-5-[4-(methylsulfonyl)phenyl]-2-phenyloxazole (B-193);

4-(4-fluorophenyl)-2-methyl-5-[4-(methylsulfonyl)phenyl]oxazole (B-194);

4-[5-(3-fluoro-4-methoxyphenyl)-2-trifluoromethyl-4-oxazolyl]benzenesulfonamide (B-195);

6-chloro-7-(1,1-dimethylethyl)-2-trifluoromethyl-2H-1-benzopyran-3-carboxylic acid (B-196);

6-chloro-8-methyl-2-trifluoromethyl-2H-1-benzopyran-3-carboxylic acid (B-197);

5,5-dimethyl-3-(3-fluorophenyl)-4-methylsulfonyl-2(5H)-furanone (B-198);

6-chloro-2-trifluoromethyl-2H-1-benzothiopyran-3-carboxylic acid (B-199);

4-[5-(4-chlorophenyl)-3-(trifluoromethyl)-1H-pyrazol-1-yl]benzenesulfonamide (B-200);

4-[5-(4-methylphenyl)-3-(trifluoromethyl)-1H-pyrazol-1-yl]benzenesulfonamide (B-201);

4-[5-(3-fluoro-4-methoxyphenyl)-3-(difluoromethyl)-1H-pyrazol-1-yl]benzenesulfonamide (B-202);

3-[1-[4-(methylsulfonyl)phenyl]-4-trifluoromethyl-1H-imidazol-2-yl]pyridine (B-203);

2-methyl-5-[1-[4-(methylsulfonyl)phenyl]-4-trifluoromethyl-1H-imidazol-2-yl]pyridine (B-204);

4-[2-(5-methylpyridin-3-yl)-4-(trifluoromethyl)-1H-imidazol-1-yl]benzenesulfonamide (B-205);

4-[5-methyl-3-phenylisoxazol-4-yl]benzenesulfonamide (B-206);

4-[5-hydroxymethyl-3-phenylisoxazol-4-yl]benzenesulfonamide (B-207);

[2-trifluoromethyl-5-(3,4-difluorophenyl)-4-oxazolyl]benzenesulfonamide (B-208);

4-[2-methyl-4-phenyl-5-oxazolyl]benzenesulfonamide (B-209);

4-[5-(2-fluoro-4-methoxyphenyl)-2-trifluoromethyl-4-oxazolyl]benzenesulfonamide (B-210);

[2-(2-chloro-6-fluoro-phenylamino)-5-methyl-phenyl]-acetic acid or COX 189 (lumiracoxib; B-211);

N-(4-Nitro-2-phenoxy-phenyl)-methanesulfonamide or nimesulide (B-212);

N-[6-(2,4-difluoro-phenoxy)-1-oxo-indan-5-yl]-methanesulfonamide or flosulide (B-213);

N-[6-(2,4-Difluoro-phenylsulfanyl)-1-oxo-1H-inden-5-yl]-methanesulfonamide, soldium salt or L-745337 (B-214);

N-[5-(4-fluoro-phenylsulfanyl)-thiophen-2-yl]-methanesulfonamide or RWJ-63556 (B-215);

3-(3,4-Difluoro-phenoxy)-4-(4-methanesulfonyl-phenyl)-5-methyl-5-(2,2,2-trifluoro-ethyl)-5H-furan-2-one or L-784512 or L-784512 (B-216);

(5Z)-2-amino-5-[[3,5-bis(1,1-dimethylethyl)-4-hydroxyphenyl]methylene]-4(5H)-thiazolone or darbufelone (B-217);

CS-502 (B-218);

LAS-34475 (B-219);

LAS-34555 (B-220);

S-33516 (B-221);

SD-8381 (B-222);

L-783003 (B-223);

N-[3-(formylamino)-4-oxo-6-phenoxy-4H-1-benzopyran-7-yl]-methanesulfonamide or T-614 (B-224);

D-1367 (B-225);

L-748731 (B-226);

(6aR,10aR)-3-(1,1-dimethylheptyl)-6a,7,10,10a-tetrahydro-1-hydroxy-6,6-dimethyl-6H-dibenzo[b,d]pyran-9-carboxylic acid or CT3 (B-227);

CGP-28238 (B-228);

4-[[3,5-bis(1,1-dimethylethyl)-4-hydroxyphenyl]methylene]dihydro-2-methyl-2H-1,2-oxazin-3(4H)-one or BF-389 (B-229);

GR-253035 (B-230);

6-dioxo-9H-purin-8-yl-cinnamic acid (B-231);

S-2474 (B-232);

4-[4-(methyl)-sulfonyl)phenyl]-3-phenyl-2(5H)-furanone;

4-(5-methyl-3-phenyl-4-isoxazolyl);

2-(6-methylpyrid-3-yl)-3-(4-methylsulfonylphenyl)-5-chloropyridine;

4-[5-(4-methylphenyl)-3-(trifluoromethyl)-1H-pyrazol-1-yl];

N-[[4-(5-methyl-3-phenyl-4-isoxazolyl)phenyl]sulfonyl];

4-[5-(3-fluoro-4-methoxyphenyl)-3-difluoromethyl)-1H-pyrazol-1-yl]benzenesulfonamide;

(S)-6,8-dichloro-2-(trifluoromethyl)-2H-1-benzopyran-3-carboxylic acid;

2-(3,4-difluorophenyl)-4-(3-hydroxy-3-methylbutoxy)-5-[4-(methylsulfonyl)phenyl]-3(2H)-pyridzainone;

2-trifluoromethyl-3H-naptho[2,1-b]pyran-3-carboxylic acid;

6-chloro-7-(1,1 -dimethylethyl)-2-trifluoromethyl-2H-1-benzopyran-3-carboxylic acid;

[2-(2,4-dichloro-6-ethyl-3,5-dimethyl-phenylamino)-5-propyl-phenyl]-acetic acid.

TABLE 3X
EXAMPLES OF CYCLOOXYGENASE-2 SELECTIVE
INHIBITORS AS EMBODIMENTS

| Compound | |
|---|---|
| Number | Structural Formula |
| B-26 | 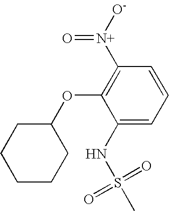 |
| | N-(2-cyclohexyloxynitrophenyl) methane |
| | sulfonamide or NS-398; |
| B-27 | 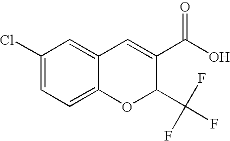 |
| | 6-chloro-2-trifluoromethyl-2H-1-benzopyran- |
| | 3-carboxylic acid; |
| B-28 | 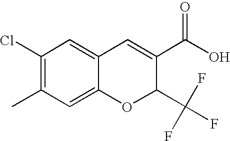 |
| | 6-chloro-7-methyl-2-trifluoromethyl-2H-1- |
| | benzopyran-3-carboxylic acid; |
| B-29 | 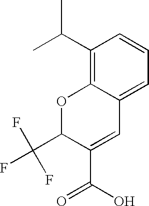 |
| | 8-(1-methylethyl)-2-trifluoromethyl-2H-1- |
| | benzopyran-3-carboxylic acid; |
| B-30 | 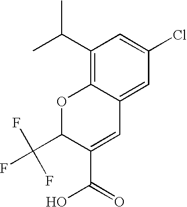 |
| | 6-chloro-8-(1-methylethyl)-2-trifluoromethyl- |
| | 2H-1-benzopyran-3-carboxylic acid; |
| B-31 | 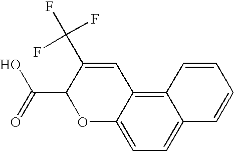 |
| | 2-trifluoromethyl-3H-naphtho[2,1-b]pyran- |
| | 3-carboxylic acid; |
| B-32 | 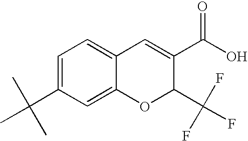 |
| | 7-(1,1-dimethylethyl)-2-trifluoromethyl-2H- |
| | 1-benzopyran-3-carboxylic acid; |
| B-33 | 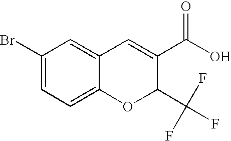 |
| | 6-bromo-2-trifluoromethyl-2H-1-benzopyran- |
| | 3-carboxylic acid; |
| B-34 | 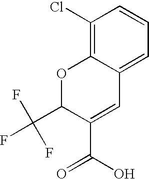 |
| | 8-chloro-2-trifluoromethyl-2H-1-benzopyran- |
| | 3-carboxylic acid; |
| B-35 | 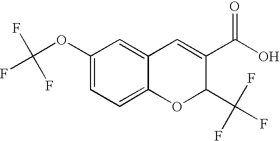 |
| | 6-trifluoromethoxy-2-trifluoromethyl-2H-1- |
| | benzopyran-3-carboxylic acid; |
| B-36 | 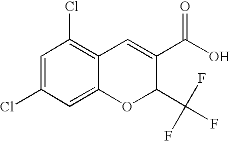 |
| | 5,7-dichloro-2-trifluoromethyl-2H-1-benzopyran- |
| | 3-carboxylic acid; |
| B-37 | 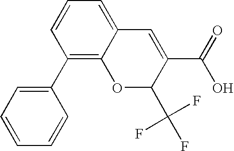 |
| | 8-phenyl-2-trifluoromethyl-2H-1-benzopyran-3- |
| | carboxylic acid; |
| B-38 | 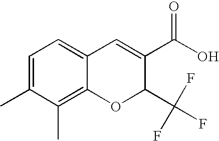 |
| | 7,8-dimethyl-2-trifluoromethyl-2H-1-benzopyran-3- |
| | carboxylic acid; |
| B-39 | 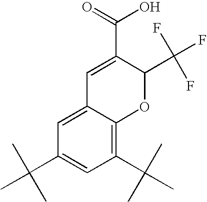 |
| | 6,8-bis(dimethylethyl)-2-trifluorometbyl-2H-1-benzopyran- |
| | 3-carboxylic acid; |
| B-40 | 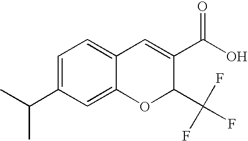 |
| | 7-(1-methylethyl)-2-trifluoromethyl-2H-1-benzopyran- |
| | 3-carboxylic acid; |
| B-41 | 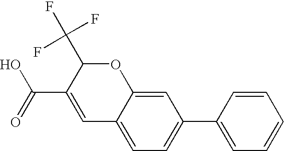 |
| | 7-phenyl-2-trifluoromethyl-2H-1-benzopyran-3- |
| | carboxylic acid; |
| B-42 | 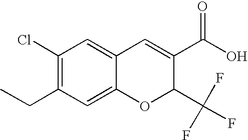 |
| | 6-chloro-7-ethyl-2-trifluoromethyl-2H-1-benzopyran- |
| | 3-carboxylic acid; |
| B-43 | 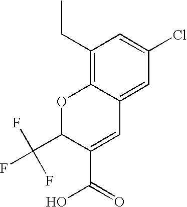 |
| | 6-chloro-8-ethyl-2-trifluoromethyl-2H-1-benzopyran- |
| | 3-carboxylic acid; |
| B-44 | 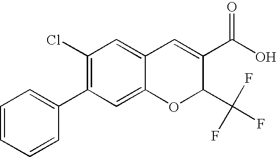 |
| | 6-chloro-7-phenyl-2-trifluoromethyl-2H-1-benzopyran- |
| | 3-carboxylic acid; |
| B-45 | 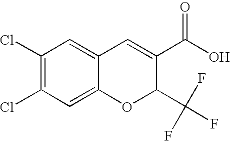 |
| | 6,7-dichloro-2-trifluoromethyl-2H-1-benzopyran- |
| | 3-carboxylic acid; |
| B-46 | 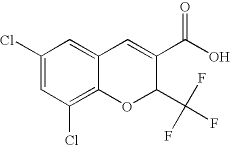 |
| | 6,8-dichloro-2-trifluoromethyl-2H-1- |
| | benzopyran-3-carboxylic acid; |
| B-47 | 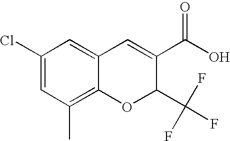 |
| | 6-chloro-8-methyl-2-trifluoromethyl-2H-1-benzopyran- |
| | 3-carboxylic acid; |
| B-48 | 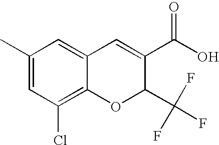 |
| | 8-chloro-6-methyl-2-trifluoromethyl-2H-1-benzopyran- |
| | 3-carboxylic acid; |
| B-49 | 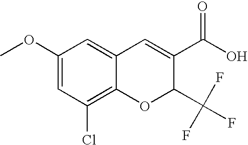 |
| | 8-chloro-6-methoxy-2-trifluoromethyl-2H-1-benzopyran- |
| | 3-carboxylic acid; |
| B-50 | 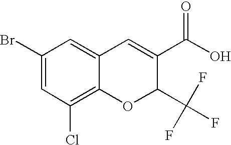 |
| | 6-bromo-8-chloro-2-trifluoromethyl-2H-1-benzopyran- |
| | 3-carboxylic acid; |
| B-51 | 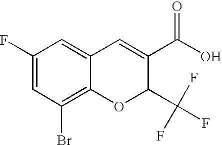 |
| | 8-bromo-6-fluoro-2-trifluoromethyl-2H-1-benzopyran- |
| | 3-carboxylic acid; |
| B-52 | 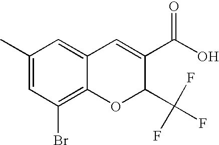 |
| | 8-bromo-6-methyl-2-trifluoromethyl-2H-1-benzopyran- |
| | 3-carboxylic acid; |
| B-53 | 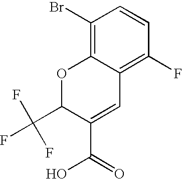 |
| | 8-bromo-5-fluoro-2-trifluoromethyl-2H-1-benzopyran- |
| | 3-carboxylic acid; |
| B-54 | 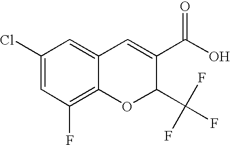 |
| | 6-chloro-8-fluoro-2-trifluoromethyl-2H-1-benzopyran- |
| | 3-carboxylic acid; |
| B-55 | 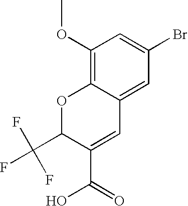 |
| | 6-bromo-8-methoxy-2-trifluoromethyl-2H-1-benzopyran- |
| | 3-carboxylic acid; |
| B-56 | 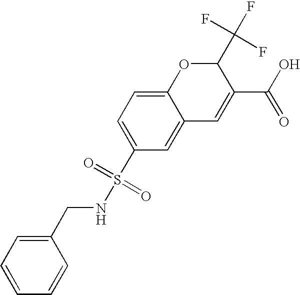 |
| | 6-[[(phenylmethyl)amino]sulfonyl]-2-trifluoromethyl- |
| | 2H-1-benzopyran-3-carboxylic acid; |
| B-57 | 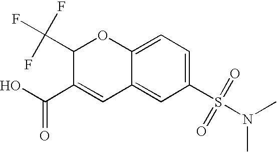 |
| | 6-[(dimethylamino)sulfonyl]-2-trifluoromethyl-2H- |
| | 1-benzopyran-3-carboxylic acid; |
| B-58 | 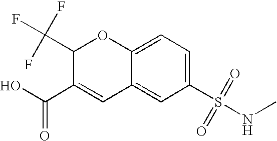 |
| | 6-[(methylamino)sulfonyl]-2-trifluoromethyl-2H-1- |
| | benzopyran-3-carboxylic acid; |
| B-59 | 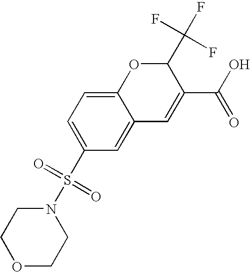 |
| | 6-[(4-morpholino)sulfonyl]-2-trifluoromethyl-2H-1- |
| | benzopyran-3-carboxylic acid; |
| B-60 | 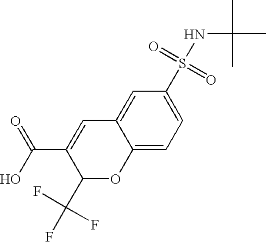 |
| | 6-[(1,1-dimethylethyl)aminosulfonyl]-2- |
| | trifluoromethyl-2H-1-benzopyran-3-carboxylic acid; |
| B-61 | 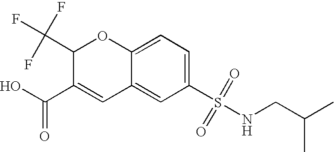 |
| | 6-[(2-methylpropyl)aminosulfonyl]-2- |
| | trifluoromethyl-2H-1-benzopyran-3-carboxylic acid; |
| B-62 | 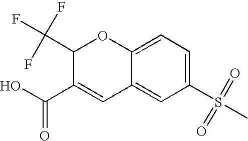 |
| | 6-methylsulfonyl-2-trifluoromethyl-2H-1- |
| | benzopyran-3-carboxylic acid; |
| B-63 | 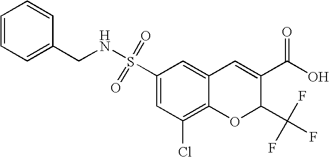 |
| | 8-chloro-6-[[(phenylmethyl)amino]sulfonyl]- |
| | 2-trifluoromethyl- |
| | 2H-1-benzopyran-3-carboxylic acid; |
| B-64 | 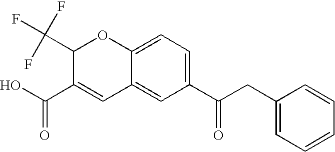 |
| | 6-phenylacetyl-2-trifluoromethyl-2H-1- |
| | benzopyran-3-carboxylic acid; |
| B-65 | 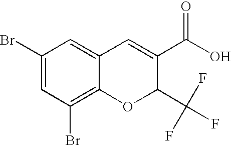 |
| | 6,8-dibromo-2-trifluoromethyl-2H-1- |
| | benzopyran-3-carboxylic acid; |
| B-66 | 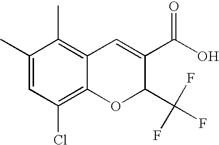 |
| | 8-chloro-5,6-dirnethyl-2-trifluoromethyl-2H- |
| | 1-benzopyran-3-carboxylic acid; |
| B-67 | 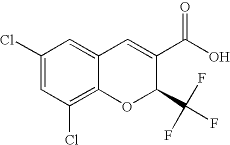 |
| | 6,8-dichloro-(S)-2-trifluoromethyl-2H-1- |
| | benzopyran-3-carboxylic acid; |
| B-68 | 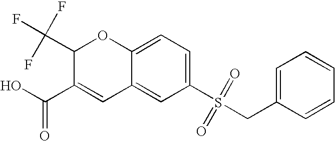 |
| | 6-benzylsulfonyl-2-trifluoromethyl-2H-1- |
| | benzopyran-3-carboxylic acid; |
| B-68 | 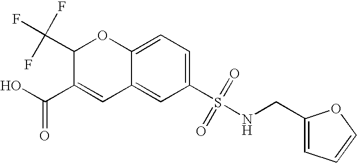 |
| | 6-[[N-(2-furylmethyl)amino]sulfonyl]-2- |
| | trifluoromethyl-2H-1-benzopyran-3-carboxylic acid; |
| B-70 | 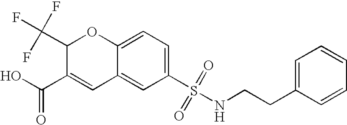 |
| | 6-[[N-(2-phenylethyl)amino]sulfonyl]-2- |
| | trifluoromethyl-2H-1-benzopyran-3-carboxylic acid; |
| B-71 | 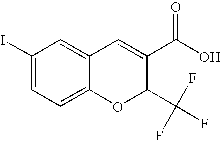 |
| | 6-iodo-2-trifluoromethyl-2H-1-benzopyran-3- |
| | carboxylic acid; |
| B-72 | 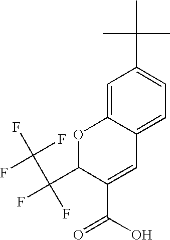 |
| | 7-(1,1-dimethylethyl)-2-pentafluoroethyl-2H-1- |
| | benzopyran-3-carboxylic acid, |
| B-73 | 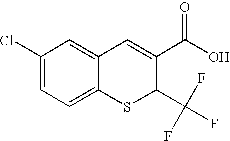 |
| | 6-chloro-2-trifluoromethyl-2H-1-benzothiopyran- |
| | 3-carboxylic acid; |
| B-74 | 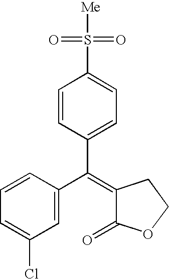 |
| | 3-[(3-chloro-phenyl)-(4-methanesulfonyl-phenyl)- |
| | methylene]-dihydro-furan-2-one or BMS-347070; |
| B-75 | 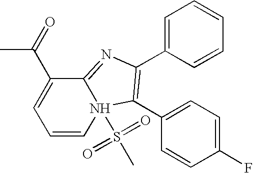 |
| | 8-acetyl-3-(4-fluorophenyl)-2-(4-methylsulfonyl)phenyl- |
| | imidazo(1,2-a)pyridine; |
| B-76 | 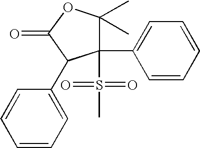 |
| | 5,5-dimethyl-4-(4-methylsulfonyl)phenyl-3- |
| | phenyl-2-(5H)-furanone; |
| B-77 | 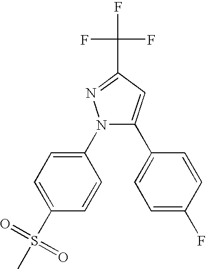 |
| | 5-(4-fluorophenyl)-1-[4-(methylsulfonyl)phenyl]- |
| | 3-(trifluoromethyl)pyrazole; |
| B-78 | 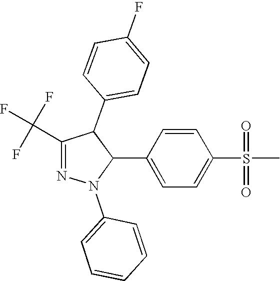 |
| | 4-(4-fluorophenyl)-5-[4-(metbylsulfonyl)phenyl]- |
| | 1-phenyl-3-(trifluoromethyl)pyrazole; |
| B-79 | 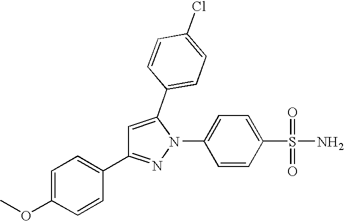 |
| | 4-(5-(4-chlorophenyl)-3-(4-methoxyphenyl)-1H- |
| | pyrazol-1-yl)benzenesulfonamide; |
| B-80 | 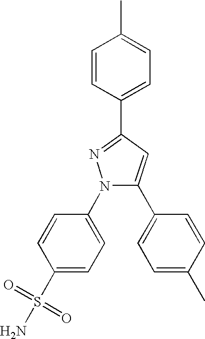 |
| | 4-(3,5-bis(4-methylphenyl)-1H-pyrazol-1- |
| | yl)benzenesulfonamide; |
| B-81 | 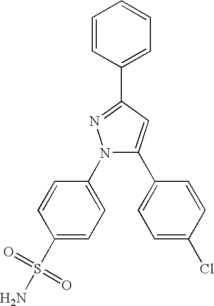 |
| | 4-(5-(4-chlorophenyl)-3-phenyl-1H-pyrazol- |
| | 1-yl)benzenesulfonamide; |
| B-82 | 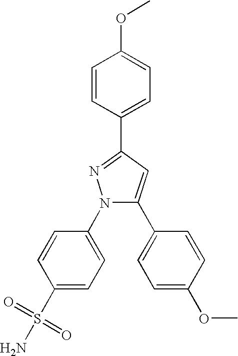 |
| | 4-(3,5-bis(4-methoxyphenyl)-1H-pyrazol- |
| | 1-yl)benzenesulfonamide; |
| B-83 | 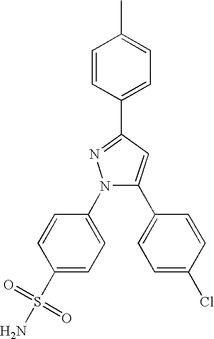 |
| | 4-(5-(4-chlorophenyl)-3-(4-methylphenyl)-1H- |
| | pyrazol-1-yl)benzenesulfonamide; |
| B-84 | 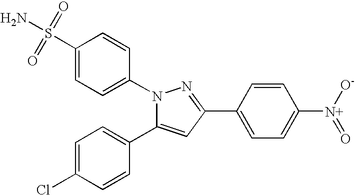 |
| | 4-(5-(4-chlorophenyl)-3-(4-nitrophenyl)-1H- |
| | pyrazol-1-yl)benzenesulfonamide; |
| B-85 | 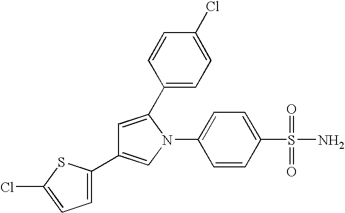 |
| | 4-(5-(4-chlorophenyl)-3-(5-chloro-2-thienyl)- |
| | 1H-pyrazol-1-yl)benzenesulfonamide; |
| B-86 | 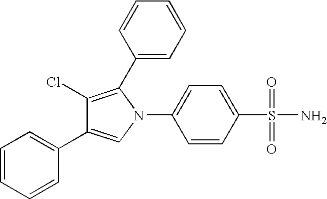 |
| | 4-(4-chloro-3,5-diphenyl-1H-pyrazol-1- |
| | yl)benzenesulfonamide; |
| B-87 | 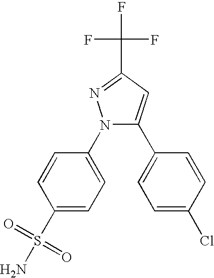 |
| | 4-[5-(4-chlorophenyl)-3-(trifluoromethyl)-1H- |
| | pyrazol-1-yl]benzenesulfonamide; |
| B-88 | 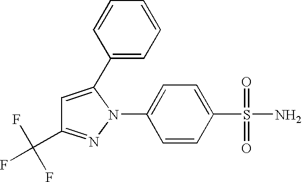 |
| | 4-[5-phenyl-3-(trifluoromethyl)-1H-pyrazol- |
| | 1-yl]benzenesulfonamide; |
| B-89 | 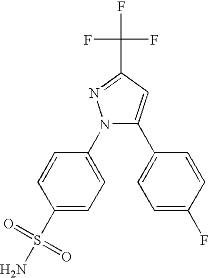 |
| | 4-[5-(4-fluorophenyi)-3-(trifluoromethyl)- |
| | 1H-pyrazol-1-yl]benzenesulfonamide; |
| B-90 | 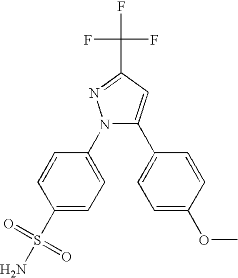 |
| | 4[5-(4-methoxyphenyl)-3-(trifluoromethyl)- |
| | 1H-pyrazol-1-yl]benzenesulfonamide; |
| B-91 | 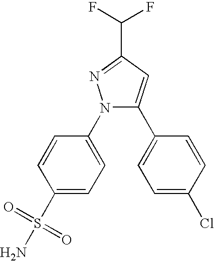 |
| | 4-[5-(4-chlorophenyl)-3-(difluoromethyl)-1H- |
| | pyrazol-1-yl]benzenesulfonamide; |
| B-92 | 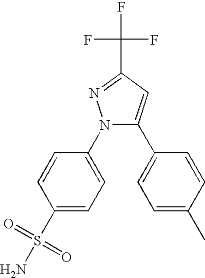 |
| | 4-[5-(4-methylphenyl)-3-(trifluoromethyl)-1H- |
| | pyrazol-1-yl]benzenesulfonamide; |
| B-93 | 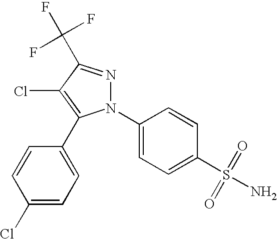 |
| | 4-[4-chloro-5-(4-chlorophenyl)-3-(trifluoromethyl)- |
| | 1H-pyrazol-1-yl]benzenesulfonamide; |
| B-94 | 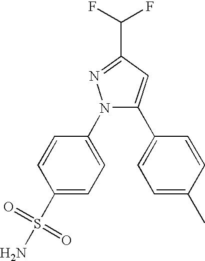 |
| | 4-[3-(difluoromethyl)-5-(4-methylphenyl)-1H- |
| | pyrazol-1-yl]benzenesulfonamide; |
| B-95 | 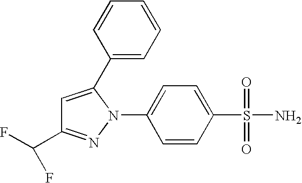 |
| | 4-[3-(difluoromethyl)-5-phenyl-1H-pyrazol- |
| | 1-yl]benzenesulfonamide; |
| B-96 | 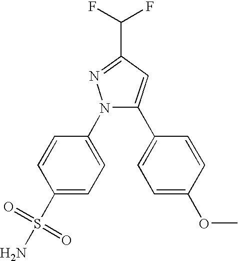 |
| | 4-[3-(difluoromethyl)-5-(4-methoxyphenyl)-1H- |
| | pyrazol-1-yl]benzenesulfonamide; |
| B-97 | 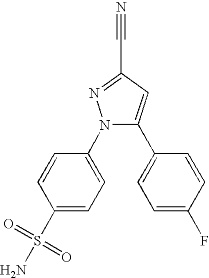 |
| | 4-[3-cyano-5-(4-fluorophenyl)-1H-pyrazol- |
| | 1-yl]benzenesulfonamide; |
| B-98 | 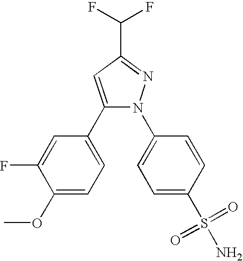 |
| | 4-[3-(difluoromethyl)-5-(3-fluoro-4- |
| | methoxyphenyl)-1H-pyrazol-1-yl] |
| | benzenesulfonamide; |
| B-99 | 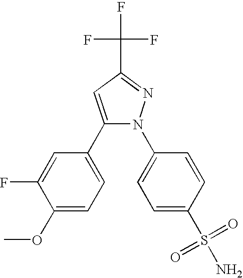 |
| | 4-[5-(3-fluoro-4-methoxyphenyl)-3- |
| | (trifluoromethyl)-1H-pyrazol-1-yl] |
| | benzenesulfonamide; |
| B-100 | 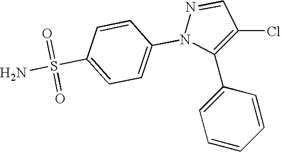 |
| | 4-[4-chloro-5-phenyl-1H-pyrazol- |
| | 1-yl]benzenesulfonamide; |
| B-101 | 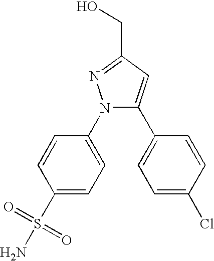 |
| | 4-[5-(4-chlorophenyl)-3-(hydroxymethyl)-1H- |
| | pyrazol-1-yl]benzenesulfonamide; |
| B-102 | 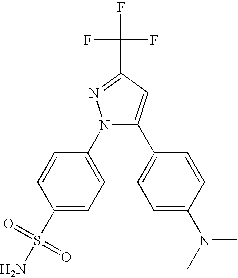 |
| | 4-[5-(4-(N,N-dimethylamino)phenyl)-3- |
| | (trifluoromethyl)-1H-pyrazol-1- |
| | yl]benzenesulfonamide; |
| B-103 | 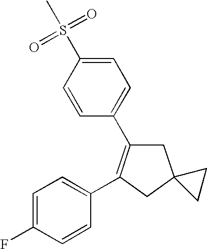 |
| | 5-(4-fluorophenyl)-6-[4- |
| | (methylsulfonyl)phenyl]spiro[2.4]hept-5-ene; |
| B-104 | 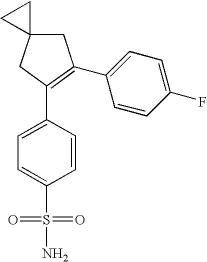 |
| | 4-[6-(4-fluorophenyl)spiro[2.4]hept- |
| | 5-en-5-yl]benzenesulfonamide; |
| B-105 | 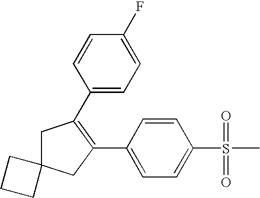 |
| | 6-(4-fluorophenyl)-7-[4- |
| | methylsulfonyl)phenyl]spiro[3.4]oct-6-ene; |
| B-106 | 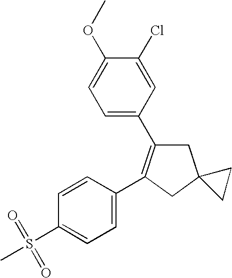 |
| | 5-(3-chloro-4-methoxyphenyl)-6-[4- |
| | (methylsulfonyl)phenyl]spiro[2.4]hept-5-ene; |
| B-107 | 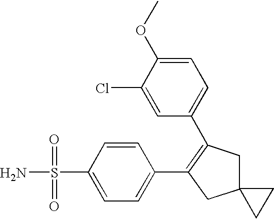 |
| | 4-[6-(3-chloro-4-methoxyphenyl)spiro[2.4]hept- |
| | 5-en-5-yl]benzenesulfonamide; |
| B-108 | 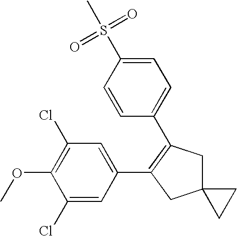 |
| | 5-(3,5-dichloro-4-methoxyphenyl)-6-[4- |
| | (methylsulfonyl)phenyl] |
| | spiro[2.4]hept-5-ene; |
| B-109 | 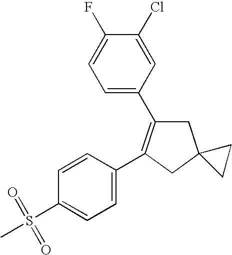 |
| | 5-(3-chloro-4-fluorophenyl)-6-[4- |
| | (methylsulfonyl)phenyl]spiro[2.4]hept-5-ene; |
| B-110 | 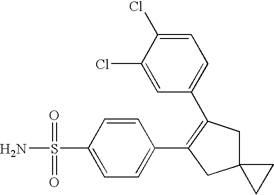 |
| | 4-[6-(3,4-dichlorophenyl)spiro[2.4]hept-5- |
| | en-5-yl]benzenesulfonamide; |
| B-111 | 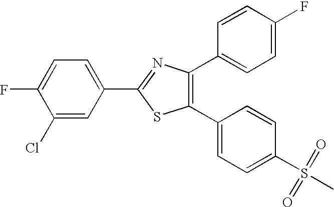 |
| | 2-(3-chloro-4-fluorophenyl)-4-(4-fluorophenyl)- |
| | 5-(4-methylsulfonylphenyl)thiazole; |
| B-112 | 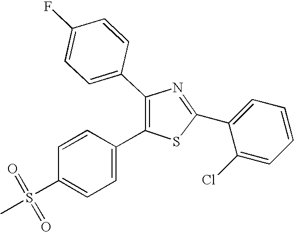 |
| | 2-(2-chlorophenyl)-4-(4-fluorophenyl)-5- |
| | (4-methylsulfonylphenyl)thiazole; |
| B-113 | 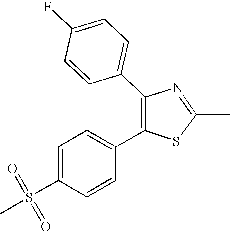 |
| | 5-(4-fluorophenyl)-4-(4-methylsulfonylphenyl)- |
| | 2-methylthiazole; |
| B-114 | 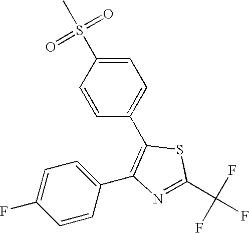 |
| | 4-(4-fluorophenyl)-5-(4-methylsulfonylphenyl)- |
| | 2-trifluoromethylthiazole; |
| B-115 | 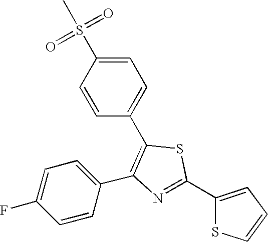 |
| | 4-(4-fluorophenyl)-5-(4-methylsulfonylphenyl)- |
| | 2-(2-thienyl)thiazole; |
| B-116 | 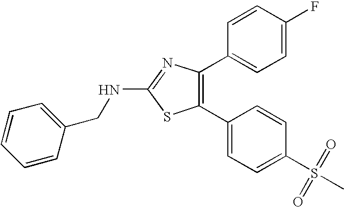 |
| | 4-(4-fluorophenyl)-5-(4-methylsulfonylphenyl)- |
| | 2-benzylaminothiazole; |
| B-117 | 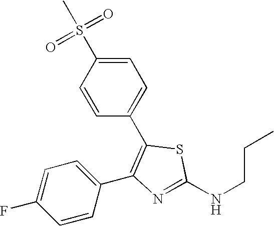 |
| | 4-(4-fluorophenyl)-5-(4-methylsulfonylphenyl)- |
| | 2-(1-propylamino)thiazole; |
| B-118 | 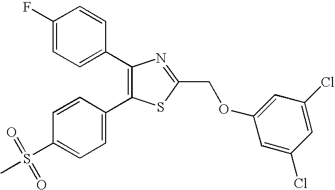 |
| | 2-((3,5-dichlorophenoxy)methyl)-4-(4- |
| | fluorophenyl)-5-[4-(methylsulfonyl)phenyl]thiazole; |
| B-119 | 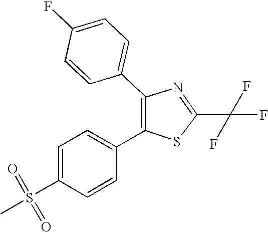 |
| | 5-(4-fluorophenyl)-4-(4-methylsulfonylphenyl)- |
| | 2-trifluoromethylthiazole; |
| B-120 | 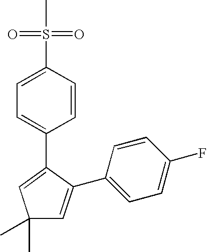 |
| | 1-methylsulfonyl-4-[1,1-dimethyl-4-(4-fluorophenyl) |
| | cyclopenta-2,4-dien-3-yl]benzene; |
| B-121 | 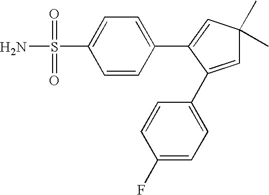 |
| | 4-[4-(4-fluorophenyl)-1,1-dimethylcyclopenta-2,4- |
| | dien-3-yl]benzenesulfonamide; |
| B-122 | 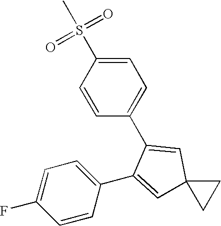 |
| | 5-(4-fluorophenyl)-6-[4-(methylsulfonyl)phenyl] |
| | spiro[2.4]hepta-4,6-diene; |
| B-123 | 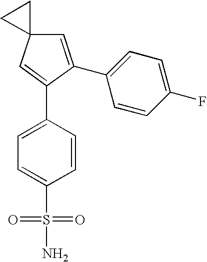 |
| | 4-[6-(4-fluorophenyl)spiro[2.4]hepta-4,6- |
| | dien-5-yl]benzenesulfonamide; |
| B-124 | 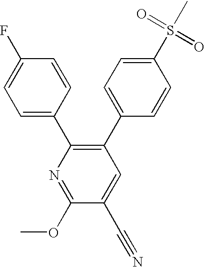 |
| | 6-(4-fluorophenyl)-2-methoxy-5-[4- |
| | (methylsulfonyl)phenyl]-pyridine- |
| | 3-carbonitrile; |
| B-125 | 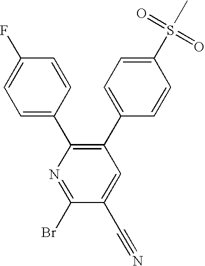 |
| | 2-bromo-6-(4-fluorophenyl)-5-[4- |
| | (methylsulfonyl)phenyl]-pyridine- |
| | 3-carbonitrile; |
| B-126 | 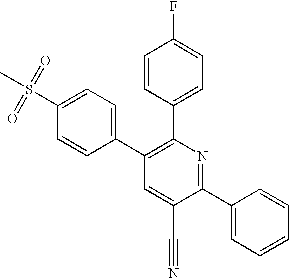 |
| | 6-(4-fluorophenyl)-5-[4- |
| | (methylsulfonyl)phenyl]-2-phenyl- |
| | pyridine-3-carbonitrile; |
| B-127 | 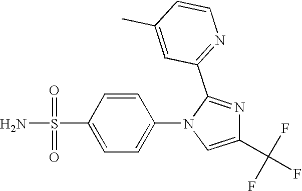 |
| | 4-[2-(4-methylpyndin-2-yl)-4-(trifluoromethyl)- |
| | 1H-imidazol-1-yl] |
| | benzenesulfonamide; |
| B-128 | 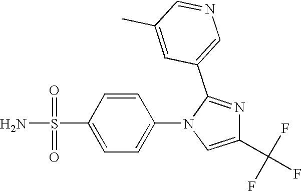 |
| | 4-[2-(5-methylpyridin-3-yl)-4-(trifluoromethyl)- |
| | 1H-imidazol-1-yl]benzenesulfonamide; |
| B-129 | 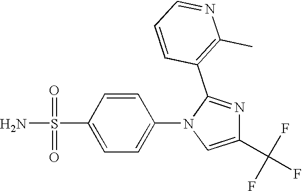 |
| | 4-[2-(2-methylpyridin-3-yl)-4-(trifluoromethyl)- |
| | 1H-imidazol-1-yl]benzenesulfonamide; |
| B-130 | 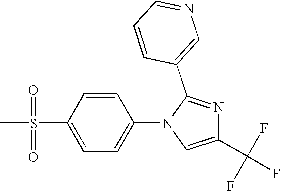 |
| | 3-[1-[4-(metbylsulfonyl)phenyl]-4- |
| | (trifluoromethyl)-1H-imidazol-2-yl]pyridine; |
| B-131 | 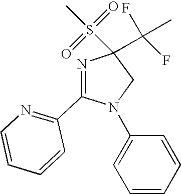 |
| | 2-[1-[4-(methylsulfonyl)phenyl-4- |
| | (trifluoromethyl)]-1H-imidazol-2-yl]pyridine; |
| B-132 | 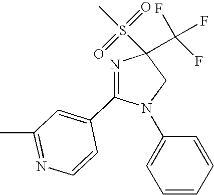 |
| | 2-methyl-4-[1-[4-(methylsulfonyl)phenyl- |
| | 4-(trifluoromethyl)]-1H-imidazol-2-yl]pyridine; |
| B-133 | 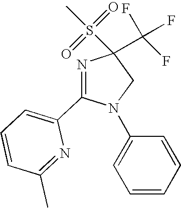 |
| | 2-methyl-6-[1-[4-(methylsulfonyl)phenyl- |
| | 4-(trifluoromethyl)]01H-imidazol-2-yl]pyridine; |
| B-134 | 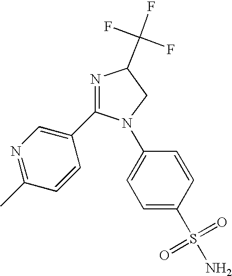 |
| | 4-[2-(6-methylpyridin-3-yl)-4-(trifluoromethyl)- |
| | 1H-imidazol-1-yl]benzenesulfonamide; |
| B-135 | 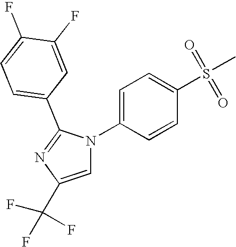 |
| | 2-(3,4-difluorophenyl)-1-[4-(methylsulfonyl)phenyl]- |
| | 4-(trifluoromethyl)-1H-imidazole; |
| B-136 | 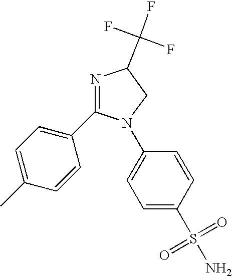 |
| | 4-[2-(4-methylphenyl)-4-(trifluoromethyl)-1H-imidazol- |
| | 1-yl]benzenesulfonamide; |
| B-137 | 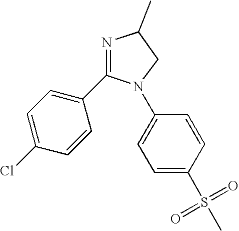 |
| | 2-(4-chlorophenyl)-1-[4-(methylsulfonyl)phenyl]- |
| | 4-methyl-1H-imidazole; |
| B-138 | 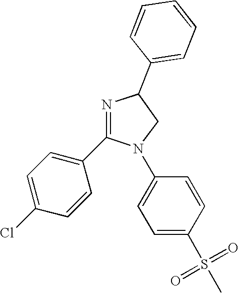 |
| | 2-(4-chlorophenyl)-1-[4-(methylsulfonyl)phenyl]- |
| | 4-phenyl-1H-imidazole; |
| B-139 | 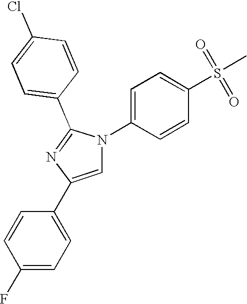 |
| | 2-(4-chlorophenyl)-4-(4-fluorophenyl)-1-[4- |
| | (methylsulfonyl)phenyl]-1H-imidazole; |
| B-140 | 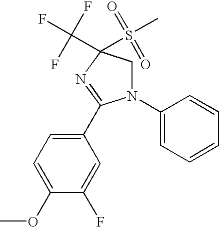 |
| | 2-(3-fluoro-4-methoxyphenyl)-1-[4-(methylsulfonyl) |
| | phenyl-4-(trifluoromethyl)]-1H-imidazole; |
| B-141 | 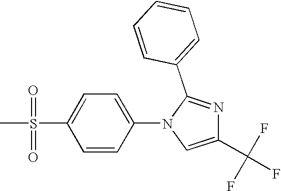 |
| | 1-[4-(methylsulfonyl)phenyl]-2-phenyl-4- |
| | trifluoromethyl-1H-imidazole; |
| B-142 | 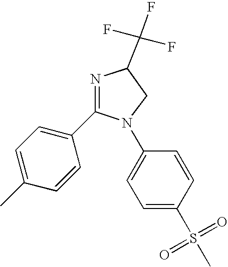 |
| | 2-(4-methylphenyl)-1-[4-(methylsulfonyl)phenyl]- |
| | 4-trifluoromethyl-1H-imidazole; |
| B-143 | 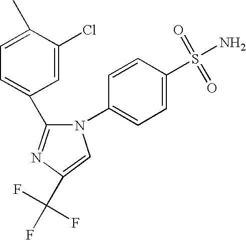 |
| | 4-[2-(3-chloro-4-methylphenyl)-4-(trifluoromethyl)- |
| | 1H-imidazol-1-yl]benzenesulfonamide; |
| B-144 | 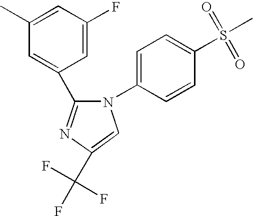 |
| | 2-(3-fluoro-5-methylphenyl)-1-[4-(methylsulfonyl)phenyl]- |
| | 4-(trifluoromethyl)-1H-imidazole; |
| B-145 | 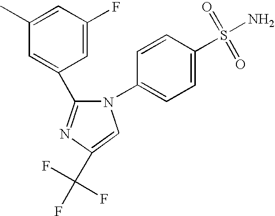 |
| | 4-[2-(3-fluoro-5-methylphenyl)-4-(trifluoromethyl- |
| | 1H-imidazole-1-yl]benzenesulfonamide; |
| B-146 | 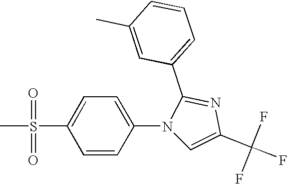 |
| | 2-(3-methylphenyl)-1-[4-(methylsulfonyl)phenyl]- |
| | 4-trifluoromethyl-1H-imidazole; |
| B-147 | 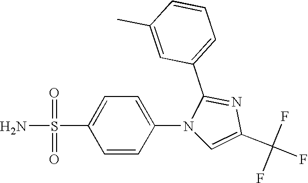 |
| | 4-[2-(3-methylphenyl)-4-trifluoromethyl-1H- |
| | imidazol-1-yl]benzenesulfonamide; |
| B-148 | 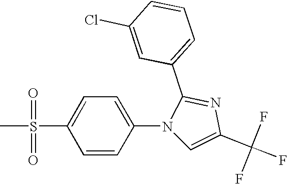 |
| | 1-[4-(methylsulfonylphenyl]-2-(3-chlorophenyl)-4- |
| | trifluoramethyl-1H-imidazole |
| B-149 | 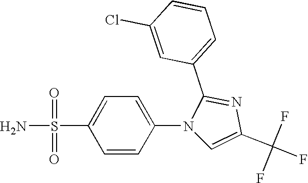 |
| | 4-[2-(3-chlorophenyl)-4-trifluoromethyl-1H- |
| | imidazol-1-yl]benzenesulfonamide; |
| B-150 | 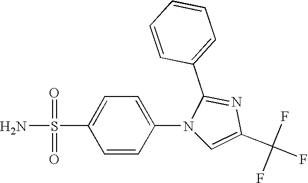 |
| | 4-[2-phenyl-4-trifluoromethyl-1H-imidazol-1- |
| | yl]benzenesulfonamide; |
| B-151 | 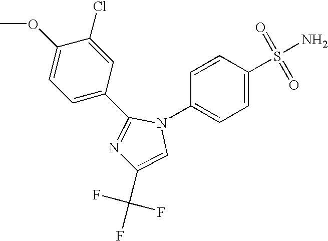 |
| | 4-[2-(4-methoxy-3-chlorophenyl)-4-trifluoromethyl-1H- |
| | imidazol-1-yl]berizenesulfonamide; |
| B-152 | 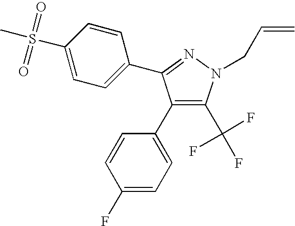 |
| | 1-allyl-4-(4-fluorophenyl)-3-[4-(methylsulfonyl)phenyl]- |
| | 5-(trifluoromethyl)-1H-pyrazole; |
| B-153 | 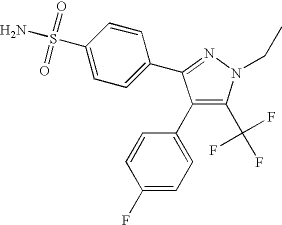 |
| | 4-[1-ethyl-4-(4-fluorophenyl)-5-(trifluoromethyl)- |
| | 1H-pyrazol-3-yl]benzenesulfonamide; |
| B-154 | 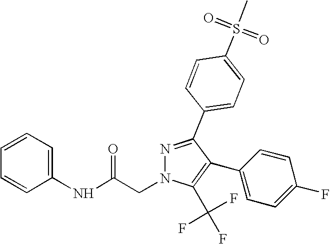 |
| | N-phenyl-[4-(4-fluorophenyl)-3-[4- |
| | (methylsulfonyl)phenyl]-5- |
| | (trifluoromethyl)-1H-pyrazol-1-yl]acetamide; |
| B-155 | 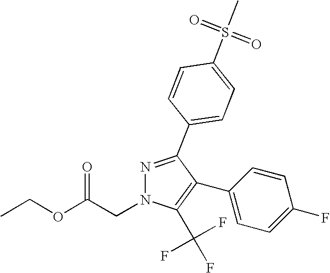 |
| | ethyl[4-(4-fluorophenyl)-3-[4- |
| | (methylsulfanyl)phenyl]-5- |
| | (trifluoromethyl)-1H-pyrazol-1-yl]acetate; |
| B-156 | 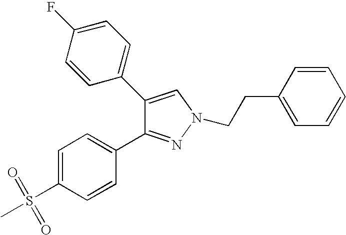 |
| | 4-(4-fluorophenyl)-3-[4-(methylsulfonyl)phenyl]- |
| | 1-(2-phenylethyl)-1H-pyrazole; |
| B-157 | 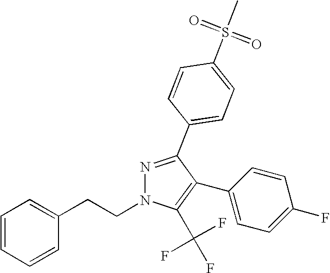 |
| | 4-(4-fluorophenyl)-3-[4-(methylsulfonyl)pheny]- |
| | 1-(2-phenylethyl)-5-(trifluoromethyl)pyrazole; |
| B-158 | 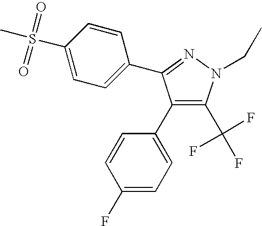 |
| | 1-ethyl-4-(4-fluorophenyl)-3-[4- |
| | methylsulfonyl)phenyl]-5- |
| | (trifluoromethyl)-1H-pyrazole; |
| B-159 | 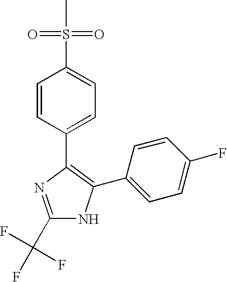 |
| | 5-(4-fluorophenyl)-4-(4-methylsulfonylphenyl)- |
| | 2-trifluoromethyl-1H-imidazole; |
| B-160 | 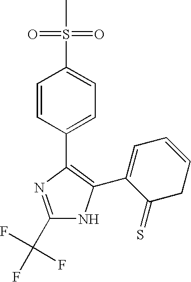 |
| | 4-[4-(methylsulfonyl)phenyl]-5-(2-thiophenyl)- |
| | 2-(trifluoromethyl)-1H-imidazole; |
| B-161 | 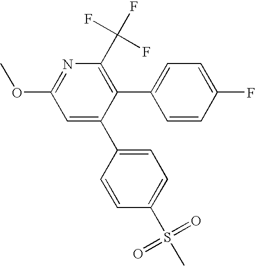 |
| | 5-(4-fluorophenyl)-2-methoxy-4-[4- |
| | (methylsulforiyl)phenyl]-6- |
| | (trifluoramethyl)pyridine; |
| B-162 | 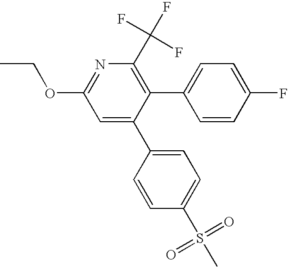 |
| | 2-ethoxy-5-(4-fluorophenyl)-4-[4- |
| | (methylsulfonyl)phenyl]-6- |
| | (trifluoromethyl)pyridine; |
| B-163 | 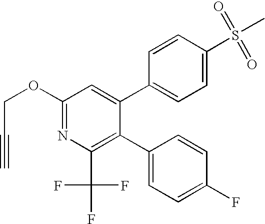 |
| | 5-(4-fluorophenyl)-4-[4-(methylsulfonyl)phenyl]- |
| | 2-(2-propynyloxy)-6-(trifluoromethyl)pyridine; |
| B-164 | 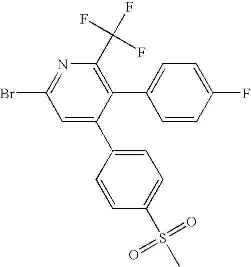 |
| | 2-bromo-5-(4-fluorophenyl)-4-[4-(methylsulfonyl) |
| | phenyl]-6-(trifluoromethyl)pyridine; |
| B-165 | 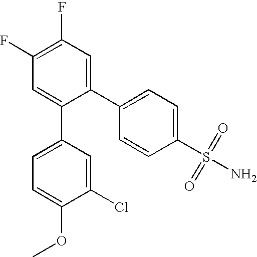 |
| | 4-[2-(3-chloro-4-methoxyphenyl)-4,5- |
| | difluorophenyl]benzenesulfonamide; |
| B-166 | 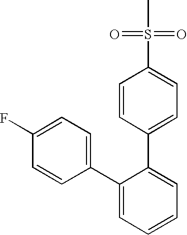 |
| | 1-(4-fluorophenyl)-2-[4- |
| | methylsulfonyl)phenyl]benzene; |
| B-167 | 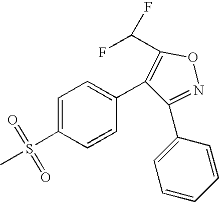 |
| | 5-difluoromethyl-4-(4-methylsulfonylphenyl)- |
| | 3-phenylisoxazole; |
| B-168 | 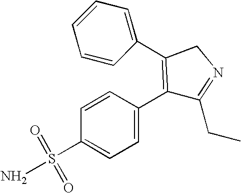 |
| | 4-[3-ethyl-5-phenylisoxazol-4- |
| | yl]benzenesulfonamide; |
| B-169 | 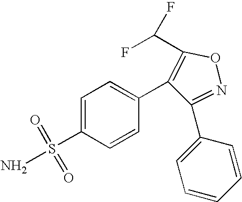 |
| | 4-[5-difluoromethyl-3-phenylisoxazol-4- |
| | yl]benzenesulfonamide; |
| B-170 | 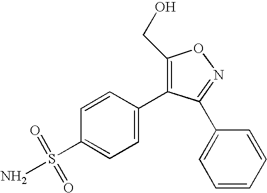 |
| | 4-[5-hydroxymethyl-3-phenylisoxazol-4- |
| | yl]benzenesulfonamide; |
| B-171 | 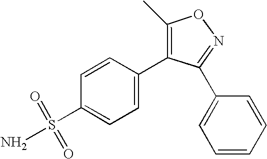 |
| | 4-[5-methyl-3-phenyl-isoxazol-4- |
| | yl]benzenesulfonamide; |
| B-172 | 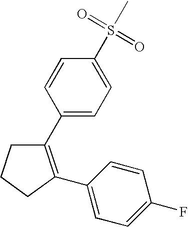 |
| | 1-[2-(4-fluorophenyl)cyclopenten-1- |
| | yl]-4-(methylsulfonyl)benzene; |
| B-173 | 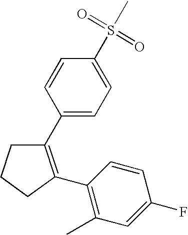 |
| | 1-[2-(4-fluoro-2-methylphenyl)cyclopenten-1-yl]- |
| | 4-(methylsulfonyl)benzene; |
| B-174 | 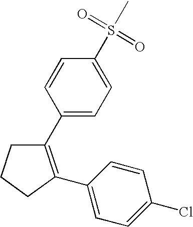 |
| | 1-[2-(4-chlorophenyl)cyclopenten-1-yl]- |
| | 4-(methylsulfonyl)benzene; |
| B-175 | 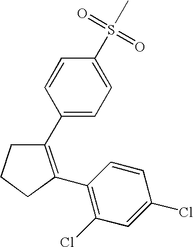 |
| | 1-[2-(2,4-dichlorophenyl)cyclopenten-1- |
| | yl]-4-(methylsulfonyl)benzene; |
| B-176 | 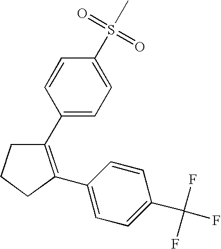 |
| | 1-[2-(4-trifloromethylphenyl)cyclopenten- |
| | 1-yl]-4-(methylsulfonyl)benzene; |
| B-177 | 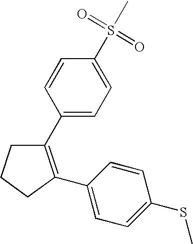 |
| | 1-[2-(4-methylthiophenyl)cyclopenten-1-yl]- |
| | 4-(methylsulfonyl)benzene; |
| B-178 | 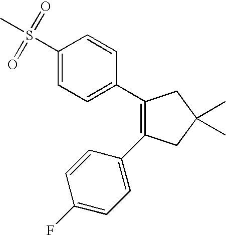 |
| | 1-[2-(4-fluorophenyl)-4,4-dimethylcyclopenten- |
| | 1-yl]-4-(methylsulfonyl)benzene; |
| B-179 | 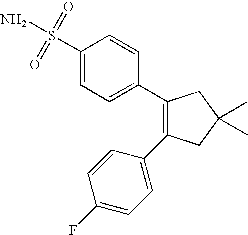 |
| | 4-[2-(4-fluorophenyl)-4,4-dimethylcyclopenten- |
| | 1-yl]benzenesulfonamide; |
| B-180 | 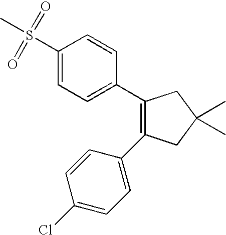 |
| | 1-[2-(3-chlorophenyl)-4,4-dimethylcyclopenten- |
| | 1-yl]-4-(methylsulfonyl)benzene; |
| B-181 | 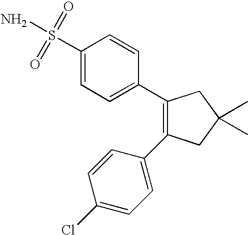 |
| | 4-[2-(4-chlorophenyl)-4,4-dimethylcyclopenten- |
| | 1-yl]benzenesulfonamide; |
| B-182 | 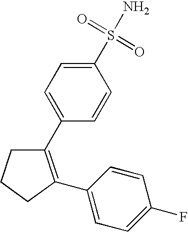 |
| | 4-[2-(4-fluorophenyl)cyclopenten-1- |
| | yl]benzenesulfonamide; |
| B-183 | 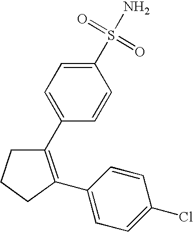 |
| | 4-[2-(4-chlorophenyl)cyclopenten-1- |
| | yl]benzenesulfonamide; |
| B-184 | 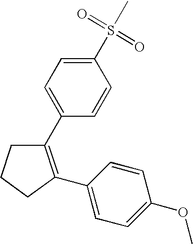 |
| | 1-[2-(4-methoxyphenyl)cyclopenten-1- |
| | yl]-4-(methylsulfonyl)benzene; |
| B-185 | 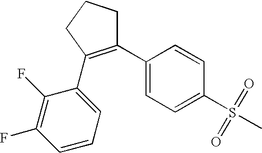 |
| | 1-[2-(2,3-difluorophenyl)cyclopenten-1-yl]- |
| | 4-(methylsulfonyl)benzene; |
| B-186 | 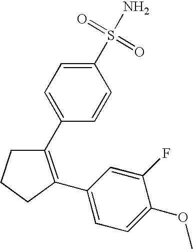 |
| | 4-[2-(3-fluoro-4-methoxyphenyl)cyclopenten- |
| | 1-yl]benzenesulfonamide; |
| B-187 | 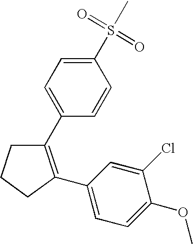 |
| | 1-[2-(3-chloro-4-methoxyphenyl)cyclopenten-1- |
| | yl]-4-(methylsulfonyl)benzene; |
| B-188 | 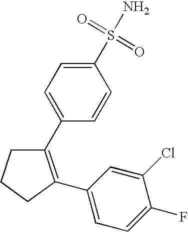 |
| | 4-[2-(3-chloro-4-fluorophenyl)cyclopenten-1- |
| | yl]benzenesulfonamide; |
| B-189 | 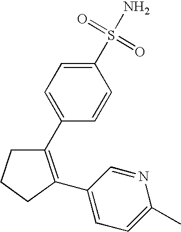 |
| | 4-[2-(2-methylpyridin-5-yl)cyclopenten-1- |
| | yl]benzenesulfonamide; |
| B-190 | 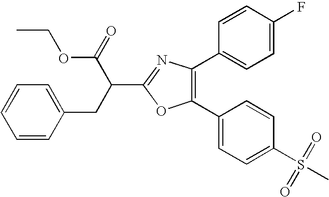 |
| | ethyl 2-[4-(4-fluorophenyl)-5-[4- |
| | (methylsulfonyl)phenyl]oxazol-2-yl]-2-benzyl-acetate; |
| B-191 | 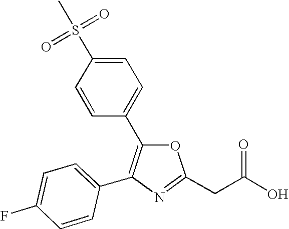 |
| | 2-[4-(4-fluorophenyl)-5-[4- |
| | (methylsulfonyl)phenyl]oxazol-2-yl]acetic acid; |
| B-192 | 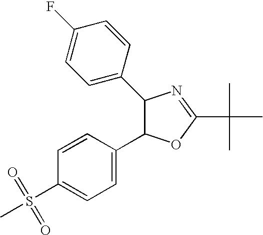 |
| | 2-(tert-butyl)-4-(4-fluorophenyl)-5-[4- |
| | (methylsulfonyl)phenyl]oxazole; |
| B-193 | 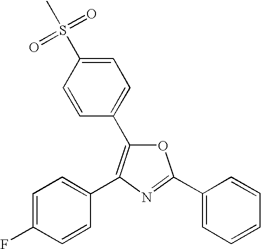 |
| | 4-(4-fluorophenyl)-5-[4-(methylsulfonyl)phenyl]- |
| | 2-phenyloxazole; |
| B-194 | 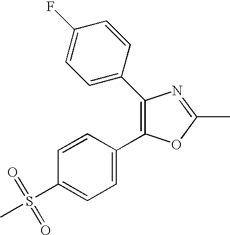 |
| | 4-(4-fluorophenyl)-2-methyl-5-[4- |
| | (methylsulfonyl)phenyl]oxazole; |
| B-195 | 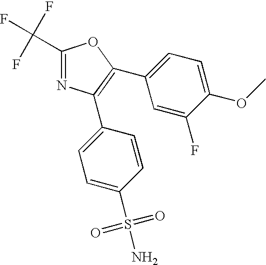 |
| | 4-[5-(3-fluoro-4-methoxyphenyl)-2-trifluoromethyl- |
| | 4-oxazolyl]benzenesulfonamide; |
| B-196 | 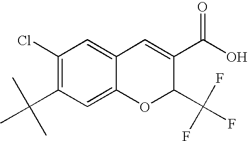 |
| | 6-chloro-7-(1,1-dimethylethyl)-2-trifluoromethyl-2H- |
| | 1-benzopyran-3-carboxylic acid; |
| B-197 | 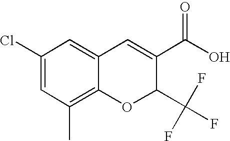 |
| | 6-chloro-8-methyl-2-trifluoromethyl-2H-1- |
| | benzopyran-3-carboxylic acid; |
| B-198 | 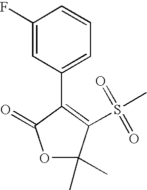 |
| | 5,5-dimethyl-3-(3-fluorophenyl)-4- |
| | methylsulfonyl-2(5H)-furanone; |
| B-199 | 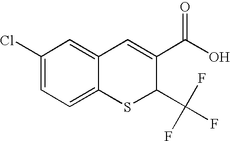 |
| | 6-chloro-2-trifluoromethyl-2H-1-benzothiopyran- |
| | 3-carboxylic acid; |
| B-200 | 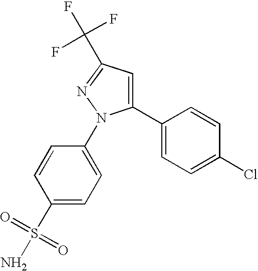 |
| | 4-[5-(4-chlorophenyl)-3-(trifluoromethyl)-1H- |
| | pyrazol-1-yl]benzenesulfonamide; |
| B-201 | 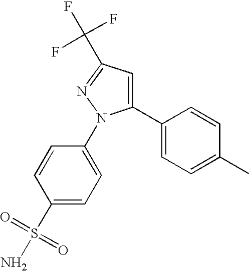 |
| | 4-[5-(4-methylphenyl)-3-(trifluoromethyl)-1H- |
| | pyrazol-1-yl]benzenesulfonamide; |
| B-202 | 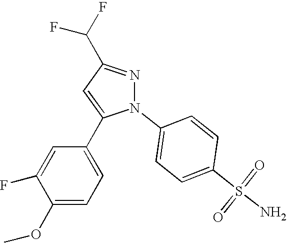 |
| | 4-[5-(3-fluoro-4-methoxyphenyl)-3-(difluoromethyl)- |
| | 1H-pyrazol-1-yl]benzenesulfonamide; |
| B-203 | 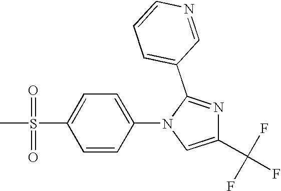 |
| | 3-[1-[4-(methylsulfonyl)phenyl]-4- |
| | trifluoromethyl-1H-imidazol-2-yl]pyridine; |
| B-204 | 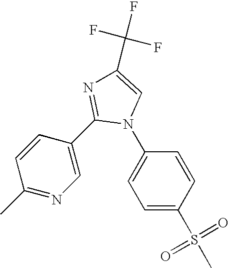 |
| | 2-methyl-5-[1-[4-(methylsulfonyl)phenyl[- |
| | 4-trifluoromethyl-1H-imidazol-2-yl]pyridine; |
| B-205 | 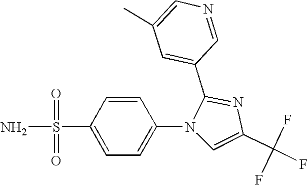 |
| | 4-[2-(5-methylpyridin-3-yl)-4-(trifluoromethyl)- |
| | 1H-imidazol-1-yl]benzenesulfonamide; |
| B-206 | 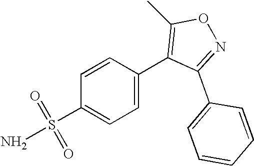 |
| | 4-[5-methyl-3-phenylisoxazol-4- |
| | yl]benzenesulfonamide; |
| B-207 | 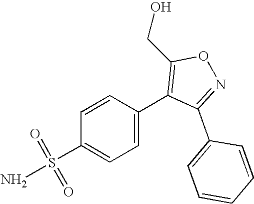 |
| | 4-[5-hydroxymethyl-3-phenylisoxazol-4- |
| | yl]benzenesulfonamide; |
| B-208 | 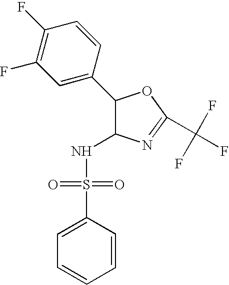 |
| | [2-trifluoromethyl-5-(3,4-difluorophenyl)-4- |
| | oxazolyl]benzenesulfonamide; |
| B-209 | 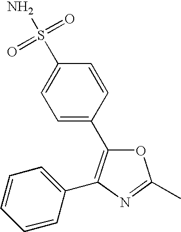 |
| | 4-[2-methyl-4-phenyl-5- |
| | oxazolyl]benzenesulfonamide; |
| B-210 | 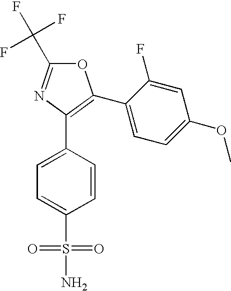 |
| | 4-[5-(2-fluoro-4-methoxyphenyl)-2- |
| | trifluoromethyl-4-oxazolyl] |
| | benzenesulfonamide; |
| B-211 | 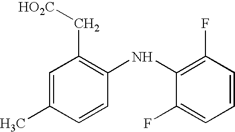 |
| B-212 | 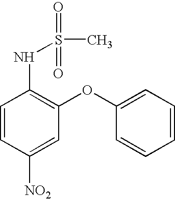 |
| | N-(4-nitro-2-phenoxy-phenyl)- |
| | methanesulfonamide or Nimesulide |
| B-213 | 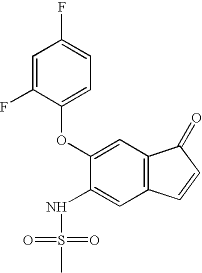 |
| | N-[6-(2,4-difluoro-phenoxy)-1-oxo-inden-5- |
| | yl]-methanesulfonamide or Flosulide |
| B-214 | 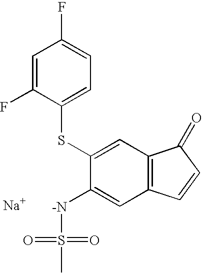 |
| | N-[6-(2,4-difluoro-phenylsulfanyl)-1-oxo-1H- |
| | inden-5-yl]-methanesulfonamide, soldium salt, |
| | or L-745337 |
| B-215 | 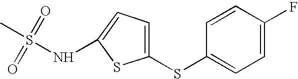 |
| | N-[5-(4-fluoro-phenylsulfanyl)-thiophen-2- |
| | yl]-methanesulfonamide or RWJ-63556 |
| B-216 | 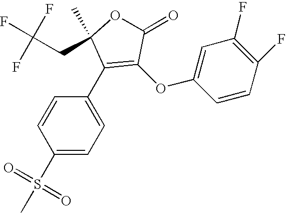 |
| | 3-(3,4-difluoro-phenoxy)-4-(4-methanesulfonyl- |
| | phenyl)-5-methyl-5-(2,2,2-trifluoro- |
| | ethyl)-5H-furan-2-one or L-784512 |
| B-217 | 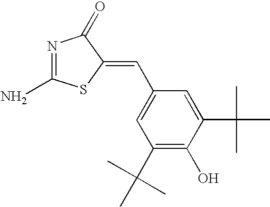 |
| | (5Z)-2-amino-5-[[3,5-bis(1,1-dimethylethyl)- |
| | 4-hydroxyphenyl]methylene]-4(5H)- |
| | thiazolone or Darbufelone |
| B-218 | CS-502 |
| B-219 | LAS-34475 |
| B-220 | LAS-34555 |
| B-221 | S-33516 |
| B-222 | SD-8381 |
| B-223 | L-783003 |
| B-224 | 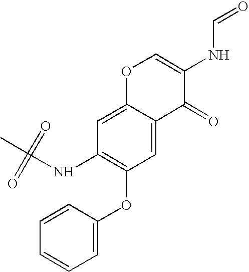 |
| | N-[3-(formylamino)-4-oxo-6-phenoxy-4H-1-benzopyran- |
| | 7-yl]-methanesulfonamide or T614 |
| B-225 | D-1367 |
| B-226 | L748731 |
| B-227 | 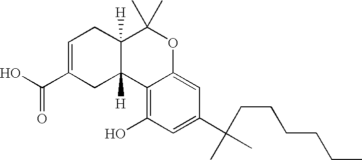 |
| | (6aR,10aR)-3-(1,1-dimethylheptyl)-6a,7,10,10a-tetrahydro- |
| | 1-hydroxy-6,6-dimethyl-6H-dibenzo[b,d] |
| | pyran-9-carboxylic acid or CT3 |
| B-228 | CGP-28238 |
| B-229 | 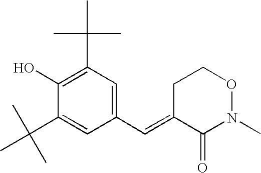 |
| | 4-[[3,5-bis(1,1-dimethylethyl)-4-hydroxyphenyl] |
| | methylene]dihydro-2-methyl-2H-1,2- |
| | oxazin-3(4H)-one or BF-389 |
| B-230 | GR-253035 |
| B-231 | 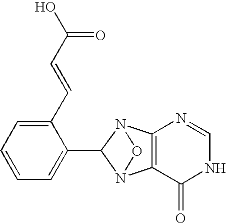 |
| | 2-(6-dioxo-9H-purin-8-yl)cinnamic acid |
| B-232 | S-2474 |
| B-233 | 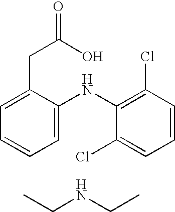 |
| B-234 | 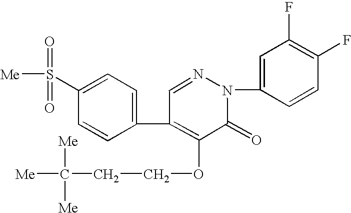 |
| B-235 | 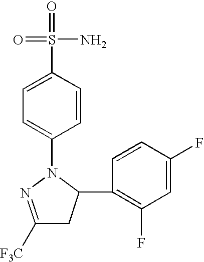 |
| B-236 | 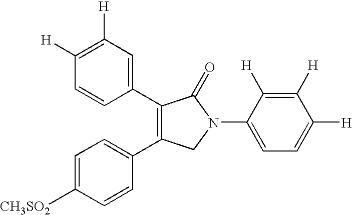 |
| B-237 | 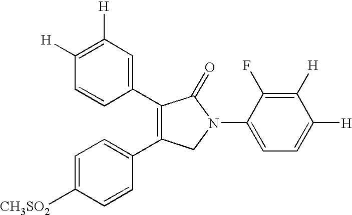 |
| B-238 | 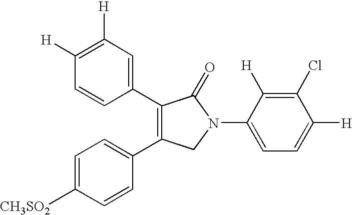 |
| B-239 | 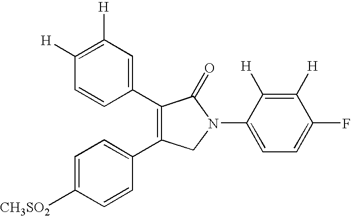 |
| B-240 | 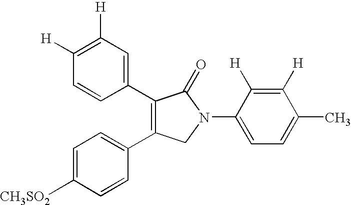 |
| B-241 | 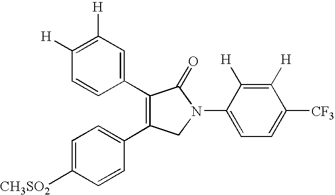 |
| B-242 | 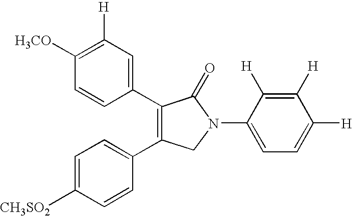 |
| B-243 | 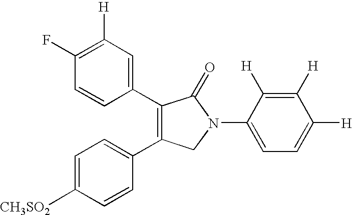 |
| B-244 | 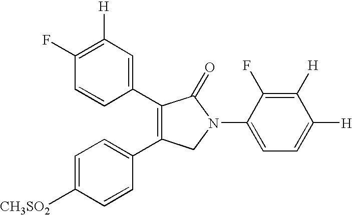 |
| B-245 | 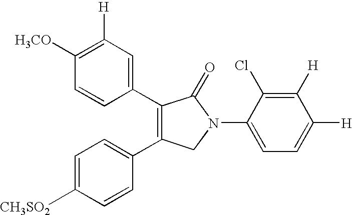 |
| B-246 | 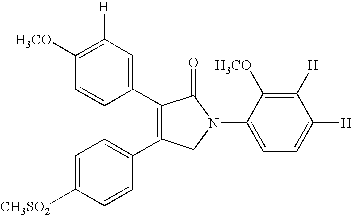 |
| B-247 | 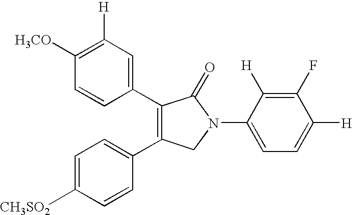 |
| B-248 | 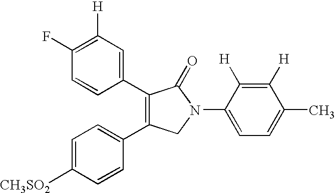 |
| B-249 | 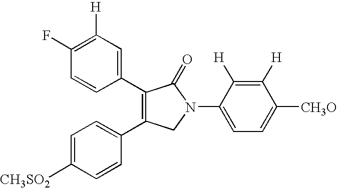 |
| B-250 | 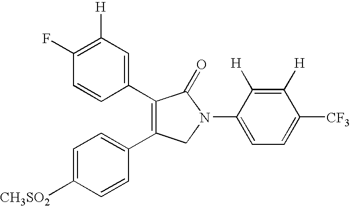 |
| B-251 | 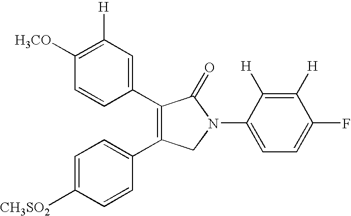 |
| B-252 | 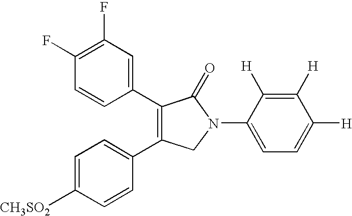 |

The cyclooxygenase-2 selective inhibitor employed in the present invention can exist in tautomeric, geometric or stereoisomeric forms. Generally speaking, suitable cyclooxygenase-2 selective inhibitors that are in tautomeric, geometric or stereoisomeric forms are those compounds that inhibit cyclooxygenase-2 activity by about 25%, more typically by about 50%, and even more typically, by about 75% or more when present at a concentration of 100 μM or less. The present invention contemplates all such compounds, including cis- and trans-geometric isomers, E- and Z-geometric isomers, R— and S-enantiomers, diastereomers, d-isomers, l-isomers, the racemic mixtures thereof and other mixtures thereof. Pharmaceutically acceptable salts of such tautomeric, geometric or stereoisomeric forms are also included within the invention. The terms “cis” and “trans”, as used herein, denote a form of geometric isomerism in which two carbon atoms connected by a double bond will each have a hydrogen atom on the same side of the double bond (“cis”) or on opposite sides of the double bond (“trans”). Some of the compounds described contain alkenyl groups, and are meant to include both cis and trans or “E” and “Z” geometric forms. Furthermore, some of the compounds described contain one or more stereocenters and are meant to include R, S, and mixtures or R and S forms for each stereocenter present.

The cyclooxygenase-2 selective inhibitors utilized in the present invention may be in the form of free bases or pharmaceutically acceptable acid addition salts thereof. The term “pharmaceutically-acceptable salts” are salts commonly used to form alkali metal salts and to form addition salts of free acids or free bases. The nature of the salt may vary, provided that it is pharmaceutically acceptable. Suitable pharmaceutically acceptable acid addition salts of compounds for use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric and galacturonic acid. Suitable pharmaceutically-acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, for example, the appropriate acid or base with the compound of any Formula set forth herein.

The cyclooxygenase-2 selective inhibitors of the present invention can be formulated into pharmaceutical compositions and administered by a number of different means that will deliver a therapeutically effective dose. Such compositions can be administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

Suppositories for rectal administration of the compounds discussed herein can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

Solid dosage forms for oral administration may include capsules, tablets, pills, powders, and granules. In such solid dosage forms, the compounds are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of active ingredient that can be combined with the carrier materials to produce a single dosage of the cyclooxygenase-2 selective inhibitor will vary depending upon the patient and the particular mode of administration. In general, the pharmaceutical compositions may contain a cyclooxygenase-2 selective inhibitor in the range of about 0.1 to 2000 mg, more typically, in the range of about 0.5 to 500 mg and still more typically, between about 1 and 200 mg. A daily dose of about 0.01 to 100 mg/kg body weight, or more typically, between about 0.1 and about 50 mg/kg body weight and even more typically, from about 1 to 20 mg/kg body weight, may be appropriate. The daily dose is generally administered in one to about four doses per day.

In one embodiment, when the cyclooxygenase-2 selective inhibitor comprises rofecoxib, it is typical that the amount used is within a range of from about 0.15 to about 1.0 mg/day·kg, and even more typically, from about 0.18 to about 0.4 mg/day·kg.

In still another embodiment, when the cyclooxygenase-2 selective inhibitor comprises etoricoxib, it is typical that the amount used is within a range of from about 0.5 to about 5 mg/day·kg, and even more typically, from about 0.8 to about 4 mg/day·kg.

Further, when the cyclooxygenase-2 selective inhibitor comprises celecoxib, it is typical that the amount used is within a range of from about 1 to about 20 mg/day·kg, even more typically, from about 1.4 to about 8.6 mg/day·kg, and yet more typically, from about 2 to about 3 mg/day·kg.

When the cyclooxygenase-2 selective inhibitor comprises valdecoxib, it is typical that the amount used is within a range of from about 0.1 to about 5 mg/day·kg, and even more typically, from about 0.8 to about 4 mg/day·kg.

In a further embodiment, when the cyclooxygenase-2 selective inhibitor comprises parecoxib, it is typical that the amount used is within a range of from about 0.1 to about 5 mg/day·kg, and even more typically, from about 1 to about 3 mg/day·kg.

Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

Calcium Modulating Agent

In addition to a cyclooxygenase-2 selective inhibitor, the composition of the invention also includes a calcium modulating agent. A number of different calcium modulating agents may be employed in the present invention. In some embodiments, the calcium modulating agent will inhibit an increase in intracellular calcium ion levels. In other embodiments, the calcium modulating agent may bind to intracellular calcium ions and inhibit calcium from acting as an intracellular secondary messenger.

One aspect of the invention encompasses calcium modulating agents that inhibit the intracellular passage of Ca²⁺ ions through one or more calcium channels. The agent may be a calcium channel receptor antagonist or a derivative or analog of a calcium channel receptor antagonist.

In one embodiment, the calcium modulating agent inhibits the intracellular passage of Ca²⁺ ions through a voltage gated calcium channel. Voltage gated calcium channels are a diverse group of multi-subunit proteins that are composed of a pore forming subunit (α₁) with α₂δ, β, and γ auxiliary subunits. A number of isoforms have been identified for each subunit and in particular, for the α₁ subunit. In a voltage gated channel, the “opening” to allow an influx of Ca²⁺ ions into the cell requires a depolarization to a certain level of the potential difference between the inside of the cell bearing the channel and the extracellular medium bathing the cell. The voltage gated calcium channel may be high-voltage activated (HVA), low-voltage activated (LVA) or a any combination thereof. Generally speaking, in a human subject, calcium channels that are considered LVA typically open in response to a depolarization of less than about 25 mV. Calcium channels that are considered HVA, on-the-other-hand, typically open in response to a depolarization of greater than about 25 mV and more typically, greater than about 50 mV. HVA and LVA channels are further classified as L-type, N-type, P/Q-type, R-type or T-type based upon each channel's particular biophysical and pharmacological properties. Representative properties for each type of channel are shown in Table 4.

TABLE 4
PROPERTIES OF THE DIFFERENT CHANNEL TYPES

| | | | | Voltage- | | Single |
|---|---|---|---|---|---|---|
| | | | | Dependent | Steady-State | Channel |
| | | | Gene | Inactivation | Inactivation | Conductance |
| Name | Type | α₁ subunit | Symbol | During Step | V50 (mV) | (pS) |
| L | HVA | α_{1S} | CACNA1S | None (Ca²⁺ | −20 | 24 |
| | | α_{1C} | CACNA1C | dependent) | | |
| | | α_{1D} | CACNA1D | | | |
| | | α_{1F} | CACNA1F | | | |
| N | HVA | α_{1B} | CACNA1B | Intermediate | −50 | 13-20 |
| P | HVA | α_{1A} | CACNA1A | None | −5 | 10-18 |
| Q | HVA | α_{1A} | CACNA1A | Intermediate | −45 | NA |
| R | H/LVA | α_{1E} | CACNA1E | Fast | −15 | NA |
| | | | | (τ = 20-30 ms) | | |
| T | LVA | α_{1G} | CACNA1G | Fast | −70 | 8 |
| | | α_{1L} | | (τ = 20-40 ms) | | |
| | | α_{1H} | CACNA1H | | | |

One embodiment, as detailed above, encompasses agents that inhibit calcium ion passage through a HVA channel. In one alternative of this embodiment, the agent inhibits the passage of calcium ions through a L-type channel. Typically, these agents inhibit calcium ion passage through channels resulting from the expression of α_{1C}, α_{1D}, α_{1S}, or α_{1F} genes or any isoforms thereof (embodiments of the α_{1S} subunit are shown in SEQ ID Nos. 1 and 2; an embodiment of the α_{1C} subunit is shown in SEQ ID No. 3; an embodiment of the α_{1D} subunit is shown in SEQ ID No. 4; embodiments of the α_{1F} subunit are shown in SEQ ID Nos. 5-7). In one alternative of this embodiment, the agent is a member of the dihydropyridine class of compounds. Suitable dihydropyridine compounds are shown in Table 5.

TABLE 5

| Common Name | Structure |
|---|---|
| Nimodipine | 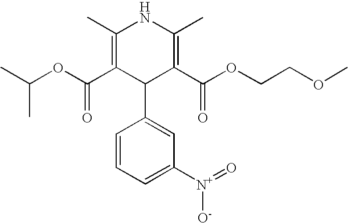 |
| Nicardipine | 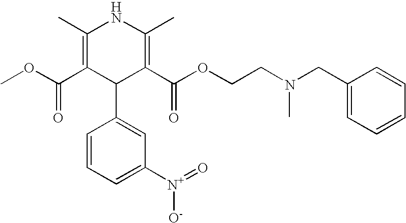 |
| Nifedipine | 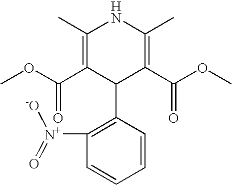 |
| Amlodipine | 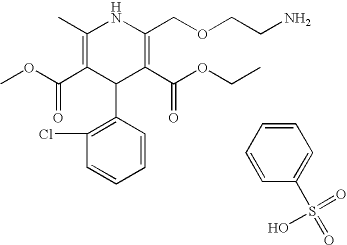 |
| Isradipine | 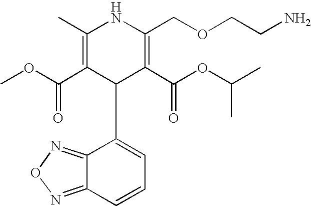 |

In another embodiment, agents belonging to the benzothiazepine class of compounds may be employed to inhibit passage of calcium ions through a L-type channel. By way of example, diltiazem, having the structure shown below, is a benzothiazepine suitable for use in the current invention.

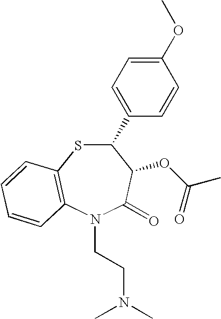

In still another embodiment, agents belonging to the diphenylalkylamine class of compounds may be employed to inhibit passage of calcium ions through a L-type channel. By way of example, verapamil, having the structure shown below, is a diphenylalkylamine suitable for use in the current invention.

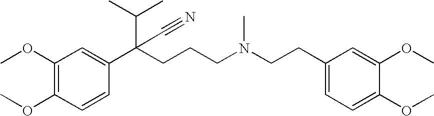

In yet another embodiment, bepridil may be employed to inhibit passage of calcium ions through a L-type channel. Bepridil has the following structure:

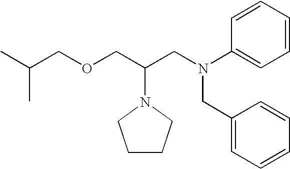

In still other embodiments, agents belonging to the piperidine class of compounds, such as those detailed in U.S. Pat. No. 5,981,539, which is hereby incorporated by reference in its entirety, may be employed to inhibit calcium ion flow through an L-type channel.

In a further alternative embodiment, the HVA gated channel is a N-type HVA channel. Generally speaking, these agents inhibit calcium ion passage through channels resulting from the expression of the α_{1B} gene or any isoforms thereof (an embodiment of the α_{1B} subunit is shown in SEQ ID No. 8). By way of example, suitable agents that inhibit the flow of calcium ions through an N-type channel include omega-conopeptides, such as ω-conotoxin GVIA (SEQ ID No:21) or ω-conotoxin MVIIA (SEQ ID No:22), which are components of peptide toxins produced by marine snails of the genus Conus. Other suitable omega-conopeptides are detailed in U.S. Pat. No. 6,156,726, which is hereby incorporated by reference in its entirety. By way of further example, neomycin sulfate or ziconotide may be employed to inhibit the flow of calcium ions through an N-type channel.

In still another alternative embodiment, the HVA gated channel a P/Q-type channel. Typically, these agents inhibit calcium ion passage through channels resulting from the expression of the α_{1A} gene or any isoforms thereof (embodiments of the α_{1A} subunit are shown in SEQ ID Nos. 9-11). Suitable agents that inhibit passage of calcium ions through a P/Q-type channel include certain isolates of funnel web spider toxin, such as agatoxin IVA (SEQ ID No:23) or agatoxin IIIA (SEQ ID No:24), and ω-conotoxin MVIIC (SEQ ID No:25).

Yet a further alternative embodiment provides agents that inhibit calcium ion passage through a R-type HVA channel. In general, these agents inhibit calcium ion passage through channels resulting from the expression of the α_{1D} gene or any isoforms thereof (embodiments of the α_{1D} subunit are shown in SEQ ID Nos. 12-14). By way of example, SNX-482 (SEQ ID No:26), a 41 amino acid peptide isolated from the venom of the African tarantula Hysterocrates gigas, may be employed to inhibit the passage of calcium ions through an R-type channel.

Another embodiment encompasses agents that inhibit calcium ion passage through a LVA gated channel. In one alternative of this embodiment, the agent inhibits the passage of calcium ions through a T-type calcium channel. Generally speaking, these agents inhibit calcium ion passage through channels resulting from the expression of α_{1G}, α_{1H}, or α_{1L} genes or any isoforms thereof (embodiments of the α_{1G} subunit are shown in SEQ ID Nos. 15-18; embodiments of the α_{1H} subunit are shown in SEQ ID Nos. 19 and 20). In one embodiment, agents belonging to the phenylalkylamine class of compounds, such as flunarizine or cinnarizine, may be employed to inhibit passage of calcium ions through a T-type channel. By way of example, a number of agents suitable for inhibiting the passage of calcium ions through a T-type channel are shown in Table 6.

TABLE 6

| Common Name | Structure |
|---|---|
| Gallopamil | 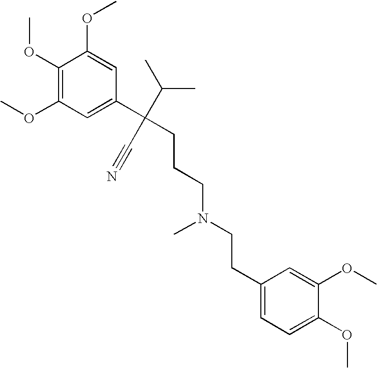 |
| Flunarizine | 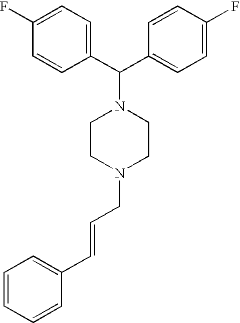 |
| Bepridil | 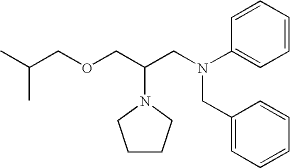 |
| Mibefradil | 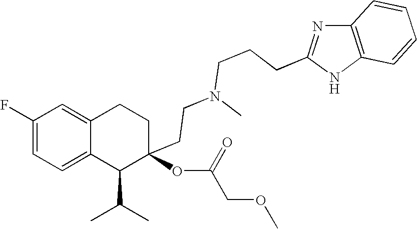 |
| Nickel | NiCl |
| Chloride | |
| Cinnarizine | 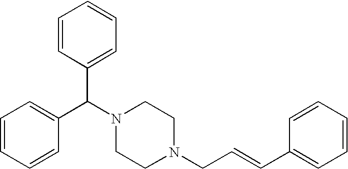 |
| Ethosuximide | 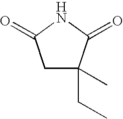 |
| Pimozide | 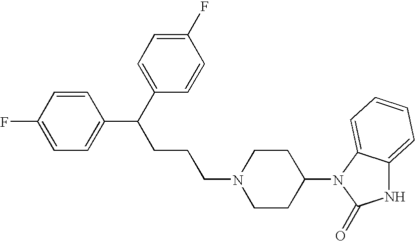 |

A further aspect of the invention encompasses calcium modulating agents that inhibit the intracellular passage of Ca²⁺ ions through a receptor operated calcium channel (ROC). Generally speaking, activation of a ROC opens a cation-selective channel that allows an influx of extracellular Ca²⁺ and Na⁺ resulting in an increase in intracellular Ca²⁺ concentration. In accordance with the practice of the invention, a number of calcium modulating agents may be employed to inhibit activation of a ROC. Typically, the agent is a ROC receptor antagonist or a derivative or analog of a calcium channel receptor antagonist.

In one embodiment, the ROC is a NMDA receptor-ionophore complex. Generally speaking, the activity of the NMDA receptor-ionophore complex is regulated by a variety of modulatory sites that can be targeted by selective antagonists. By way of example, competitive antagonists, such as the phosphonate AP5, act at the glutamate-binding site, whereas noncompetitive antagonists, such as phencyclidine (PCP), MK-801 or magnesium (Mg²⁺), act within the associated ion channel (ionophore). Alternatively, there is also a glycine-binding site that can be blocked selectively with compounds such as 7-chlorokynurenic acid. By way of further example, other potential sites for modulation of NMDA receptor function include a zinc (Zn²⁺) binding site and a sigma ligand binding site. Additionally, endogenous polyamines such as spermine bind to a specific site and so potentiate NMDA receptor function. A number of other suitable NMDA receptor antagonists are detailed in U.S. Pat. No. 6,306,912, which is hereby incorporated by reference in its entirety.

In an alternative embodiment, the ROC is a calcium-permeable AMPA receptor. The activity of the AMPA receptor is regulated by a number of modulatory sites that can be targeted by selective antagonists. By way of example, quinoxalinediones are a potent class of competitive receptor antagonists that may be employed. By way of further example, GYKI 52466, a 2,3-benzodiazepine, a highly selective, noncompetitive antagonist of AMPA/kainate receptor responses may also be employed. Additionally, a number of other suitable AMPA receptor antagonists are detailed in U.S. Pat. No. 6,306,912, which is hereby incorporated by reference in its entirety.

In still another alternative embodiment, the ROC is or a nicotinic cholinergic receptor. By way of example, passage of Ca²⁺ ions through a nicotinic cholinergic receptor may be inhibited by the arylalkylamine toxin, philanthotoxin. By way of further example, passage of Ca²⁺ ions through a nicotinic cholinergic receptor may be inhibited by mecamylamine. A number of other suitable nicotinic cholinergic receptor antagonists are detailed in U.S. Pat. No. 6,306,912, which is hereby incorporated by reference in its entirety.

A further aspect of the invention encompasses calcium modulating agents that are calcium chelating agents. Generally speaking, calcium chelating agents suitable for use in the present invention include agents that attach to Ca²⁺ ions by coordinate links to two or more nonmetal atoms in the same molecule. In some aspects, the chelating agent binds extracellular Ca²⁺ ions and inhibits its intracellular passage. In other aspects, the chelating agent binds to intracellular Ca²⁺ ions and inhibits it from functioning as a secondary.

In one embodiment, the chelating agent comprises a compound having formula X
(HOOC—CH₂—)₂—N-A-N—(—CH₂COOH)₂

wherein A is a saturated or unsaturated, aliphatic, aromatic or heterocyclic linking radical containing, in a direct chain link between the two depicted nitrogen atoms, 2-8 carbon atoms in a continuous chain which is interrupted by 2-4 oxygen atoms, provided that the chain members directly connected to the two depicted nitrogen atoms are not oxygen atoms and pharmaceutically acceptable salts of said carboxylic acids.

In a further embodiment for compounds having formula X, A is selected from the group consisting of saturated or unsaturated aliphatic chain interrupted by 2-4 oxygen atoms, and

—CR═CR—O—CH₂CH₂—O—CR′═CR′, where each of the pairs of radicals R—R and R′—R′, together with the attached —C═C— moiety, complete an aromatic or heterocyclic ring containing 5 or 6 ring atoms, the ring completed by R—R being the same as or different from that completed by R′—R′. In a further alternative for this embodiment, the aromatic or heterocyclic ring completed by the pairs of radicals R—R and R′—R′, together with the attached —C═C— moiety, is selected from the group consisting of furan, thiophene, pyrrole, pyrazole, imidazole, 1,2,3-triazole, oxazole, isoxazole, 1,2,3-oxadiazole, 1,2,5-oxadiazole, thiazole, isothiazole, 1,2,3-thiadiazole, 1,2,5-thiadiazole, benzene, pyridine, pyridazine, pyrimidine, pyrazine, 1,2,3-triazine, 1,2,4-triazine, and 1,2-, 1,3- and 1,4-oxazines and -thiazines, the ring completed by R—R being the same as or different from the ring completed by R′—R′. In still a further alternative for this embodiment, the pairs of radicals R—R and R′—R′, together with the attached —C═C— moiety, completes the same or different rings selected from unsubstituted and substituted benzene rings, in which substituted benzene rings contain 1-4 substituents selected from the group consisting of saturated or unsaturated C₁₋₄-alkyl, saturated or unsaturated C₁₋₄-alkoxy, fluorine, chlorine, bromine, iodine and CF₃, or a single divalent substituent which is —O—(CH₂)ₙ—O— and n is 1-3.

In a further embodiment for compounds having formula X, A is selected from the group consisting of —CH₂CH₂—O—CH₂CH₂—O—CH₂CH₂—, and —CH₂CH₂—(N(—CH₂COOH)—CH₂CH₂—)ₙ wherein n is 1 to 5.

In still a further embodiment for compounds having formula X, the compound is selected from the group consisting of ethylene-1,2,-diol-bis-(2-aminoethyl ether)-N,N,N′,N′-tetraacetic acid (EGTA);1,2-bis-(2-aminophenoxy)ethane-N,N,N′,N′-tetraacetic acid (BAPTA), EDTA, and DTPA.

In yet another embodiment for compounds corresponding to formula X, the compound is a di or tetra ester of a compound having formula X. In one alternative of this embodiment, the compound is a neutral lipophillic ester of EDTA, DTPA, EGTA and BAPTA.

In another embodiment, the chelating agent comprises a compound having formula XI
((HO)₂OP—CH₂—)₂—N-A-N—(—CH₂PO(OH)₂)₂

where A is saturated or unsaturated, aliphatic, aromatic or heterocyclic linking radical containing, in a direct chain link between the two depicted nitrogen atoms, 2-8 carbon atoms in a continuous chain which is interrupted by 2-4 oxygen atoms, provided that the chain members directly connected to the two depicted nitrogen atoms are not oxygen atoms and pharmaceutically acceptable salts of said phosphonic acids.

In a further embodiment for compounds having formula XI, A is selected from the group consisting of saturated or unsaturated aliphatic chain interrupted by 2-4 oxygen atoms, and —CR═CR—O—CH₂CH₂—O—CR′═CR′, where each of the pairs of radicals R—R and R′—R′, together with the attached —C═C— moiety, complete an aromatic or heterocyclic ring containing 5 or 6 ring atoms, the ring completed by R—R being the same as or different from the ring completed by R′—R′. In a further alternative for this embodiment, the aromatic or heterocyclic ring completed by the pairs of radicals R—R and R′—R′, together with the attached —C═C— moiety, is selected from the group consisting of furan, thiophene, pyrrole, pyrazole, imidazole, 1,2,3-triazole, oxazole, isoxazole, 1,2,3-oxadiazole, 1,2,5-oxadiazole, thiazole, isothiazole, 1,2,3-thiadiazole, 1,2,5-thiadiazole, benzene, pyridine, pyridazine, pyrimidine, pyrazine, 1,2,3-triazine, 1,2,4-triazine, and 1,2-, 1,3- and 1,4-oxazines and -thiazines, the ring completed by R—R being the same as or different from the ring completed by R′—R′. In still a further alternative for this embodiment, the pairs of radicals R—R and R′—R′, together with the attached —C═C— moiety, complete the same or different rings selected from unsubstituted and substituted benzene rings, in which substituted benzene rings contain 1-4 substituents selected from the group consisting of saturated or unsaturated C₁₋₄-alkyl, saturated or unsaturated C-₁₋₄-alkoxy, fluorine, chlorine, bromine, iodine and CF₃, or a single divalent substituent which is —O—(CH₂)ₙ—O— where n is 1-3.

In a further embodiment for compounds having formula XI, A is selected from the group consisting of —CH₂CH₂—O—CH₂CH₂—O—CH₂CH₂—, and —CH₂CH₂—(N(—CH₂PO(OH)₂)—CH₂CH₂—)ₙ,

wherein n is 1 to 5.

In still a further embodiment for compounds having formula XI, the compound is selected from the group consisting of ethylene-1,2,-diol-bis-(2-aminoethyl ether)-N,N,N′,N′-tetramethylenephosphonic acid (EGTMP); 1,2-bis-(2-aminophenoxy)ethane-N,N,N′,N′-tetramethylenephosphonic acid (BAPTMP); EDTMP; and DTPMP.

In yet another embodiment for compounds corresponding to formula XI, the compound is a di or tetra ester of a compound having formula X. In one alternative of this embodiment, the compound is a neutral lipophillic ester of EGTMP, BAPTMP, EDTMP or DTPMP.

In still another embodiment, the calcium chelating agent is selected from the compounds listed in Table 7.

TABLE 7

| Name | Structure |
|---|---|
| Pamidronic Acid | 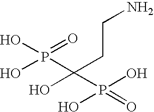 |
| Clodronic Acid | 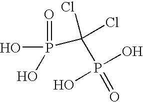 |
| Risedronic Acid | 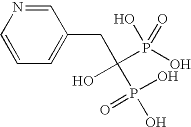 |
| Oxidronic Acid | 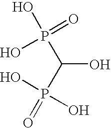 |
| Methylenediphosphonic Acid | 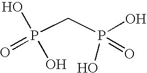 |

Examples of other suitable calcium modulating agents are detailed in Table 8.

TABLE 8

| Common Name | Trade Name | Reference |
|---|---|---|
| A-53930A (isolated from the | | JP 08208690 |
| culture medium of Streptomyces | | |
| vinaceusdrappus SANK 62394) | | |
| AE-0047 Watanidipine | | EP 00424901 |
| dihydrochloride | | |
| AGN-190604 | | Inflammation 1995, 19: 2 |
| | | (261-275) |
| AGN-190744 | | EP 372940 A2 |
| AH-1058 | | European Journal of |
| | | Pharmacology (2000), |
| | | 398(1), 107-112 |
| AHR 5360C | | European Journal of |
| | | Pharmacology (1988), |
| | | 146(2-3), 215-22 |
| AHR 12234 | | Archives Internationales de |
| | | Pharmacodynamie et de |
| | | Therapie (1989), 301: 131-50 |
| AHR-12742 | | ZA 08604522 |
| AHR-16303B | | Journal of Cardio vascular |
| | | Pharmacology (1991 Jan), |
| | | 17(1), 134-44 |
| AHR-16462B | | Drug Development |
| | | Research (1991), 22(3), |
| | | 259-71 |
| AIT 110 | | |
| AIT 111 | | |
| AJ 2615 | | WO 8601203 A1 |
| AJ-3941 | | Arzneimittel Forschung |
| | | (1996 Jun), 46(6), 567-71 |
| (+) alismol | | JP 04077420 A2 |
| AM-336 synthetic version of | synthetic version | WO9954350 |
| CVID marine cone snail venom | of the natural | |
| | omega-conotoxin | |
| AM 543 | | |
| amlodipine | Norvasc | U.S. Pat. No. 4572909 |
| (S)-(−) amlodipine | | GB 2233974 A1 |
| AN 132 | | EP 196648 A1 |
| anipamil LU 42668 | | EP 64158 A1 |
| antioquine (alkaloid from stem | | Journal of Natural Products |
| bark) | | (1992), 55(9), 1281-6 |
| AP-1067 | | IDdb 268934 |
| AQ-AH-208 | | CH 645628 A |
| AR 12456 (derivative of trapidil) | | BE 902218 A1; |
| | | Cardiovascular Drug |
| | | Reviews (1991), 9(4), |
| | | 385-97 |
| aranidipine | Bec MPC1304 | U.S. Pat. No. 4446325 |
| | Sapresta | |
| atosiban | | EP 00112809 |
| azelnidipine CS 905 | Calblock | EP 88 266922 |
| B 84439 | | EP 240828 |
| barnidipine (derivative of | Cyress, Hypoca, | U.S. Pat. No. 4220649; |
| nicardipine) | Mepirodipine, | DE 02904552 |
| | YM09730 | |
| BAY-E-6927 | | DE 2117571 |
| BAY-K-9320 | | EP 9206 |
| BAY-T-7207 | | |
| BBR-2160 | | EP 282904 A2 |
| BDF 8784 | | EP 25111 |
| belfosdil BMY 21891 SR 7037 | | EP 173041 A1 |
| bencyclane EGYT-201 | | FR 151193 |
| benipidine KW3049, Nakadipine | Caritec, Coniel | U.S. Pat. No. 4448964 |
| bepridil | angopril, Bapadin, | U.S. Pat. No. 3962238 |
| | Bepricor, | |
| | CERM1978, | |
| | Cordium, | |
| | OREG 5730, | |
| | Vascor | |
| bisaramil RGH 2957 | Yutac | WO 9622096 A1 |
| BK 129 | | Methods and Findings in |
| | | Experimental and Clinical |
| | | Pharmacology (1992), |
| | | 14(3), 175-81 |
| BMS-181102 | | EP 559569 |
| BMS-188107 | | U.S. Pat. No. 5070088 |
| BMY 20014 | | DE 3512995 A1 |
| BMY 20064 | | DE 3512995 A1 |
| BMY-43011 | | Bioorganic and Medicinal |
| | | Chemistry Letters |
| | | 1993, 3: 12 (2817-2820) |
| BN 50149 | | WO 9323082 A1 |
| BN 50175 | | WO 9323082 A1 |
| BN 50394 | | WO 9323082 A1 |
| BR 1022 | | Current Science (2002), |
| | | 83(4), 426-431 |
| BRL 3287A | | WO 9323082 A1 |
| BRL-32872 | | WO 09323024 |
| buflomedil | | U.S. Pat. No. 4326083 |
| butoprozine | | DE 2707048 |
| CAF 603 | | Organic and Bio-Organic |
| | | Chemistry (1994), (22), |
| | | 3349-52 |
| calciseptine (venom poly peptide) | | WO 2000 069900 |
| calcium antagonists | | WO 9205165 A1 |
| calcium channel antagonists | | WO 00236586; |
| | | WO 0236567 A1 |
| calcium channel blocker (L-type) | | Journal of Medicinal |
| | | Chemistry (1996), 39(15), |
| | | 2922-2938 |
| calcium channel blockers | | EP 400665 A2; |
| | | U.S. Pat. No. 4965356 |
| calcium channel blockers | | WO 9526325 |
| carvedilol | Artist, Aucardic, | U.S. Pat. No. 4503067 |
| | BM14190, | |
| | Cardiol, Carloc, | |
| | Caslot, Coreg, | |
| | Coropres, | |
| | Dilatrend, | |
| | Dilbloc, | |
| | Dimitone, | |
| | DQ2466, | |
| | Eucardic, Kredex | |
| caryachine | | British Journal of |
| | | Pharmacology (1995 Dec), |
| | | 116 (8), 3211-8 |
| CD-349 | | EP 92936 A1 |
| CD-832 | | EP 00370821 |
| CER-2 metabolite of furnipidine | 1E 7M | WO 9919302 |
| cerebrocrast | | DE 3534385 A1 |
| CERM 11956 | | EP 138684 A2 |
| CERM-12816 | | IDdb 283075 |
| CGP 22442 | | WO 9323082 |
| CGP 26797 | | WO 9323082 |
| CGP 28727 | | WO |
| CGP 32413 | | WO 9323082 |
| changrolin | | Sci. Sin. (Engl. Ed.) (1979), |
| | | 22 (10), 1220-8 |
| CHF-1521 (combination of | | |
| delapril & manidipine) | | |
| cilnidipine | Atelec, Cinalong, | U.S. Pat. No. 4672068 |
| | FRG 8653, | |
| | Siscard | |
| cinnarizine 516-MD | Stugeron | U.S. Pat. No. 3799934 |
| | Cinnaron | |
| civamide | cis-capsaicin | WO 9640079; |
| | | U.S. Pat. No. 5840762 |
| clentiazem, TA 3090 | Logna | EP 00127882; |
| | | U.S. Pat. No. 4567175 |
| clevidipine | | WO 9512578 |
| CNS-1067 | | IDdb 211675 |
| CNS-1237 | | Annals of the New York |
| | | Academy of Sciences |
| | | (1995), 765 |
| | | (Neuroprotective Agents), |
| | | 210-29 |
| CNS-2103 (from spider venom) | | WO 9214709 A2 |
| COR 28-22 | | WO 9323082 |
| COR 2707C | | WO 9323082 |
| COR 3752C | | WO 9323082 |
| CP-060S | | WO 9500471 A1 |
| CPC-301 | | IDdb 231888 |
| CPC 304 | | IDdb 185705 |
| CPC-317 | | IDdb 185700 |
| CPU 23 | | Yaoxue Xuebao (1990), |
| | | 25(11), 815-23. |
| | | CAN 114: 143097 |
| CPU-86017 | | EP 00538844 |
| CRE 202 | | WO 9323082 |
| CRE 204 | | WO 9323082 |
| CRE 1005 | | WO 9323082 |
| CRL-42752 | | WO 00003987 |
| cronidipine LF 2-0254 | | EP 240398 A1 |
| CV 159 | | FR 2511370 A1 |
| D-2024 see verapamil(S) | | WO 09509150 |
| D 2603 | | WO 9323082 |
| dagapamil | | WO 9323082; |
| | | EP 64158 A1 |
| darodipine PY 108068 | | EP 00000150 |
| dauricine NSC 36413 | | Chung-Kuo Yao Li Hsueh |
| | | Pao (Acta Pharmacologica |
| | | Sinica) (1986 Nov), 7(6), |
| | | 543-7 |
| des methyl verapamil | | |
| DHM 9 | | WO 8604581 A1 |
| DHP-218 PAK 9 | | EP 00121117 |
| diclofurime | | DE 79-2922799 |
| dihydropyridine calcium channel | | Journal of Medicinal |
| blockers | | Chemistry (1998), 41(4), |
| | | 509-514 |
| diltiazem | Cardizem, | U.S. Pat. No. 3562257 |
| | Dilacor, Tiazac | |
| diperdipine | | EP 00218996 |
| dipfluzine | | DE 3318577 A1 |
| diproteverine BRL 40015 | | BE 866208 |
| dopropidil | | EP 00031771 |
| dotarizine FI 6026 | Dotaricin, | U.S. Pat. No. 4883797 |
| DTZ-323 | | Molecular Pharmacology |
| | | (1997), 51(2), 262-268 |
| E-2050 | | JP 2001199949 A2 |
| E 4080 | | EP 344577 A2 |
| efonidipine hydrochloride | Landel, NZ105, | U.S. Pat. No. 4885284 |
| | Selefodipine | |
| EG 1088 | | EP 56637 A1 |
| EGIS 3966 | | DE 4027052 A1 |
| elgodipine | | DE 3825962 A1 |
| emopamil (racemic) SZ 45 | | DE 3344755 A1 |
| (S)-emopamil | | DE 3344755 A1 |
| enalapril + nitrendipine, Vita- | | EP 00884054 |
| Invest | | |
| etafenone LG 11457 | Baxacor | DE 1265758 |
| ethosuximide | Suxinutin, | |
| | Zardondan, | |
| | Zardontin | |
| eugenodilol | | JP 11255719 A2 |
| evodiamine | | JP 52077098 |
| F-0401 | | EP 00320984 |
| falipamil AQA 39 | | Journal of Medicinal |
| | | Chemistry (1990 May), 33 |
| | | (5), 1496-504 |
| fantofarone SR 33557 | | EP 235111 A1; |
| | | U.S. Pat. No. 4957925 |
| fasudil (iv formulation), Asahi | Erik, Fasdil, | U.S. Pat. No. 4678783 |
| | AT877 | |
| FCE-24265 | | EP 373645 A1 |
| FCE-26262 | | |
| FCE-27335 | | |
| FCE-27892 | | |
| FCE-28718 | | EP 00755931 |
| fedopamil | | |
| felodipine | Lexxel Plendil | U.S. Pat. No. 4264611 |
| felodipine + ramipril, | | WO 09607400 |
| Astra/Aventis | | |
| fendiline | Sensit Cordan | U.S. Pat. No. 3262977 |
| | (HCl) | |
| feniline | | |
| flezelastine, D 18024 | | EP 590551 A2 |
| flordipine | | |
| flunarizine | Sibelium | U.S. Pat. No. 3773939 |
| fluodipine | | Arzneimittel-Forschung |
| | | (1992), 42(11), 1284-7 |
| fluphenazine, S94; SQ 4918; | Elinol, Pacinol, | JOURNAL OF |
| Triflumethazine; Vespazine | Siqualon, | MEDICINAL |
| | Valamina | CHEMISTRY (1976 Jun), |
| | | 19(6), 850-2 |
| fostedil KB944 | | EP 10120 |
| FPL 62129 | | EP 125803 A2 |
| FR 46171 | | |
| FR-172516 | | JP 09040647 A2 |
| FRC 8411 | | |
| FRG 8653 | | |
| FRG-8701 | | |
| furaldipine | | |
| furnidipine (CRE 319) | | Journal of Medicinal |
| | | Chemistry (1995 Jul 21), 38 |
| | | (15), 2830-41 |
| gallopamil (methoxy analog of | Procurum | U.S. Pat. No. 3262977 |
| verapamil) | | |
| GOE 5057 | | |
| GOE 5584A | | EP 173933 A1 |
| GOE 93007 | | |
| GR 60139 | | |
| GR 66234A R-enantiomer of | | Haematologica (1994), |
| telupidine | | 79(4), 328-33 |
| GR 66235A, L-enantiomer of | | Haematologica (1994), |
| telupidine | | 79(4), 328-33 |
| GS-386 | | |
| GYKI 46544 | | |
| H 32438 | | |
| HA 22 | | U.S. Pat. No. 5240947 A |
| HA 23 | | U.S. Pat. No. 5240947 |
| HA 1004 | | |
| HA 1077 | | |
| HE 30346 | | |
| HNS 32 | | JP 08311007 A2 |
| HOE 166 | | Molecular Pharmacology |
| | | (1988 Apr), 33(4), 363-9 |
| HOE 263 | | |
| HP 406 | | U.S. Pat. No. 4521537 |
| ICI 206970 | | EP 293170 A1 19881130 |
| iganidipine | | JP 63225355 A2 |
| | | 19880920 |
| IHC 72 | | YAO HSUEH HSUEH |
| | | PAO [ACTA |
| | | PHARMACEUTICA |
| | | SINICA] (1992), 27(6), |
| | | 407-11 |
| ipenoxazone | | |
| isradipine | Dynacirc | U.S. Pat. No. 4466972 |
| JTV-519 | K-201 | WO 09212148 |
| KB 2796 | | |
| KP-840 | | Yakubutsu, Seishin, Kodo |
| | | (1992), 12(6), 353 |
| KP 873 | | |
| KT-362 | | ARCHIV DER |
| | | PHARMAZIE (1995 Apr), |
| | | 328(4), 313-6 |
| KT 2230 | | GENERAL |
| | | PHARMACOLOGY |
| | | (1991), 22(3), 443-8 |
| KW 3049 (see benipidine) | | |
| L-366682 | | EP 00444898 |
| L-651582 | | |
| L 735821 | | WO 9514471 A1 |
| | | 19950601 |
| | | British Journal of |
| | | Pharmacology (2001), 132 |
| | | (1), 101-110 |
| lacidipine GR43659 SN305 | Aponil, Caldine, | U.S. Pat. No. 4801599; |
| | Lacimen, Lacipil, | DE 03529997 |
| | Midotens, | |
| | Motens, Viapress | |
| LAS 30356 | | |
| LAS 30398 | | |
| LAS-30538 | | Journal of Pharmacy and |
| | | Pharmacology (1992 Oct), |
| | | 44(10), 830-5 |
| LAS Z077 | | |
| LCB-2514 | | |
| lemildipine | | P 59152373 A2 |
| lercanidipine | Cardiovasc, | U.S. Pat. No. 4705797 |
| | Carmen, Coifeo, | |
| | Lercadip, Lerdip, | |
| | Lerzam, | |
| | REC152375, | |
| | Vasodip, Zanedip, | |
| | Zanidip | |
| leualacin | | EP 00358418 |
| levosemotiadil, SA 3212 | | WO 08700838 |
| lidoflazine R7904 | Clinium | U.S. Pat. No. 3267104 |
| lifarizine RS 87476 | | U.S. Pat. No. 04935417 |
| LOE-908 | | |
| lomerizine KB 2796 | Migsis | U.S. Pat. No. 4663325; |
| | | EP 00159566 |
| LU 49700 (main metabolite of | | DE 3642331 A1 |
| galllopamil) | | |
| LU 49938 | | |
| LY-042826 | | European Journal of |
| | | Pharmacology (2000), |
| | | 408(3), 241-248 |
| LY-393615 | | European Journal of |
| | | Pharmacology (2000), |
| | | 408(3), 241-248 |
| manidipine CV 4093, franidipine | Calslot, Iperten, | U.S. Pat. No. 4892875; |
| | Manivasc | EP 00094159 |
| MCI 176 (MY7674) | | EP 169537 A2 |
| McN 5691 (see RWJ 26240 | | |
| McN-6186 | | |
| MCN 6497 | | |
| MD 260792 | | |
| MDL 143 | | |
| MDL 12330A | | |
| MDL 16582A | | WO 9323082 |
| MDL 72567 | | GB 2137622 A1 |
| | | 19841010 |
| | | CAN 102: 95549 |
| MEM 1003 nimopidine analog | | |
| BAY Z 4406 | | |
| mepirodipine | | |
| mesudipine | | |
| mibefradil | Posicor | EP 00268148; |
| | | U.S. Pat. No. 4808605 |
| minodipine | | |
| mioflazine | | |
| MJ 14712 | | |
| monatepil maleate (AD 2615) | | WO 08601203; |
| | | U.S. Pat. No. 4749703 |
| MPC 1304 | | |
| MPC 2101 | | FR 2514761 A1 |
| MR-14134 | | Pharmacology (1995), 51 |
| | | (2), 84-95 |
| N-3601 | | EP 254322 A1 |
| N 20776 | | |
| N-allyl secoboldine | | |
| naltiazem Ro 23-6152 | | U.S. Pat. No. 4652561 |
| NB 818 | | |
| NC 1100 | | |
| NC O 700 | | |
| NCC 09-0026 | | |
| nexopamil | | EP 00271013 |
| NH 2250 | | |
| NH 2716 | | |
| nicainoprol RU 42924 | | DE 2934609 |
| nicardipine nifelan | Cardene | U.S. Pat. No. 3985847 |
| nictiazem | | |
| nifedipine | Procardia Adalat | U.S. Pat. No. 3485847 |
| | EnSo Trol | |
| nigulipine | | WO 8807525 A1 |
| niludipine | | |
| nilvadipine FK 235 | Escor Nivadil | U.S. Pat. No. 4338322 |
| | | DE 02940833 |
| nimodipine | Nimotop | U.S. Pat. No. 3842096 |
| nisoldipine Bay y 5552 | Sular Sysdor | U.S. Pat. No. 4154839 |
| nitrendipine Bay k 5009 | Baypress | U.S. Pat. No. 3799934 |
| NMDA/calcium channel | | WO 097455115 |
| antagonists, Allelix | | |
| NKY 722 | | |
| NMED 126 (MC-34D) | | WO 0145709 A1; |
| | | U.S. Pat. No. 6387897 |
| NMED 427 | | WO 0145709 A1; |
| | | U.S. Pat. No. 6387897 |
| NMED 724 | | WO 0145709 A1; |
| | | U.S. Pat. No. 6387897 |
| NMED 826 | | WO 0145709 A1; |
| | | U.S. Pat. No. 6387897 |
| NMED JM-G-10 | | WO 0145709 A1; |
| | | U.S. Pat. No. 6387897 |
| NMED 157 39-1B4 | | WO 0145709 A1; |
| | | U.S. Pat. No. 6387897 |
| NMED 160 39-45-3 | | WO 0145709 A1; |
| | | U.S. Pat. No. 6387897 |
| NNC-09-0026 | | WO 9201672 |
| NP 252 | | Life Sciences (1991), |
| | | 48(2), 183-8 |
| NS 626 | | |
| NS-638 | | U.S. Pat. No. 5314903; |
| | | EP 545845 A1 |
| NS-649 | | EP 520200 A2 |
| NS-696 | | |
| NS-7 | | WO 09607641 |
| NS 3034 | | |
| NZ 105 | | |
| olradipine S 11568 | | FR 2602231 A1 |
| ONO-2921 | | WO 0000470 A1 |
| OPC 13340 | | |
| OPC 88117 | | EP 236140 A2 |
| ORG 13020 | | |
| Org-13061 | | Fundamental & Clinical |
| | | Pharmacology (1997), |
| | | 11(5), 416-426 |
| OSAT (nifedipine) | | |
| osthole | Osthol | JP 47000430 |
| oxodipine, IQB 837V | | ES 531033 A1 |
| P 0825 | | |
| P 1268 | | |
| palonidipine hydrochloride | | EP 128010 A2 |
| PCA-50922 | | |
| PCA-50938 | | Brain Research (1997), 772 |
| | | (1, 2), 57-62 |
| PCA-50941 | | |
| PCA 50982 | | |
| PD-0204318 | | WO 9943658 A1 |
| PD-029361 | | IDdb 300520 |
| PD 122860 | | EP 206747 A2 |
| PD 151307 | | U.S. Pat. No. 6423689; |
| | | J. Med. Chem (43), 3474, |
| | | 2000 |
| PD-157667 | | U.S. Pat. No. 5767129 |
| PD-158143 | | WO 9705125 A1 |
| PD 173212 | | |
| PD 175069 | | WO 9854123 A1 |
| PD-176078 | | WO9955688; |
| | | J. Med. Chem (43), 3474, |
| | | 2000 |
| PD 181283 | | Bioorganic & Medicinal |
| | | Chemistry Letters (1999), |
| | | 9(16), 2453-2458 |
| pelanserin | | |
| perhexiline | Pexid | GB 1025578 |
| petrosynol | | Tetrahedron (1993), |
| | | 49(45), 10435-8 |
| PF 244 | | |
| PFS 1114 (EO 122) | | DE 2802208 |
| pirmenol | Pirmavar, | U.S. Pat. No. 4112103 |
| | Pimenol, CI845 | |
| pirprofurol | | |
| pimozide | | Journal of Neuroscience |
| | | (2002), 22(2), 396-403. |
| PN 200110 | | |
| PNU 156654E | | WO 9705102 A1 |
| pranidipine OPC 13340 | Acalas | EP 00145434 |
| prenylamine | Angormin | U.S. Pat. No. 3152173 |
| propiverine | Detrunorm, | DD 106643 |
| | Mictonorm, | |
| | Mictonetten, | |
| | BUP 4 | |
| ptilomycalin AM | mimic of | |
| | ptilomycalin | |
| QM 96233 | | |
| QM 96159 | | |
| QM 96127 | | |
| QX-314 | | Biophysical Journal (1979 |
| | | Jul), 27(1), 39-55. |
| R 56865 | | EP 184257 A1 |
| R 59494 | | EP 184257 A1 |
| R 71811 | | |
| Rec 152288 | | |
| Rec 152375 Rec 15/375 | | |
| RGH-2716 (TDN 345) | | EP 414421 A2 |
| RGH 2970 | | |
| riodipine | | |
| Ro-11-2933 | | EP 00523493 |
| Ro 18-3981 | | |
| Ro 40-5967 | | |
| RO 445912 dithaine derivatives of | | Biochemical Pharmacology |
| tiapamil | | (1995), 50(2), 187-96 |
| ronipamil | | |
| RS-5773 | | EP 00353032 |
| RS 93007 | | |
| RS 93522 | | U.S. Pat. No. 4595690 |
| RU-43945 | | WO 9323082 A1 |
| RWJ-22108 | | U.S. Pat. No. 04845225 |
| RWJ-22726 | | U.S. Pat. No. 04845225 |
| RWJ 26240 McN 5691 | | EP 146271 A2 |
| RWJ 26899 | | EP 237191 A1 |
| RWJ-26902 | | |
| RWJ-29009 | | EP 00493048 |
| RWJ-37868 | | WO 0048584 A2 |
| ryanodine | | |
| S-(−)-amlodipine | | |
| S 11568 | | |
| S 12967 | | ZA 9000231 A |
| S-12968 | | EP 00406502 |
| S-2150 | | EP 00615971 |
| S-312-d | | JP 03052890 |
| S 830327 | | |
| SA 2572 | | JP 63104969 A2 |
| SA 2995 | | |
| SA 3212 | | |
| sabeluzole | | EP 184257 A1 |
| safinamide | FCE 26743, | EP 400495 A1 |
| | NW 1015, | |
| | PNU 151774 | |
| sagandipine | | |
| salicylaldoxime | | Clinical and Experimental |
| | | Pharmacology and |
| | | Physiology (1999 Dec), 26 |
| | | (12), 964-9 |
| SANK-71996 | | |
| SB-201823A | | WO 09202502 |
| SB-206284A | | |
| SB 221420A | | WO 9602494 A1 |
| SB-237376 | | WO 0209761 A2 |
| SB 262470 | NPS 2143 | WO 0183546 A1 |
| SC 30552 | | |
| SDZ-249482 | | |
| selodipine | | |
| semotiadil (SD 3211) | | U.S. Pat. No. 4786635; |
| | | JP 09012576 |
| SIM 6080 | | EP 293925 A2 |
| sipatrigine | | EP 372934 A2 |
| sinomenine (active from a Chinese | | WO 0269971 A1 |
| medicinal plant) | | |
| siratiazem | | WO 09117153 |
| SKF-45675 | | |
| SKF-96365 | | European Journal of |
| | | Pharmacology (1990 Jun |
| | | 12), 188(6), 417-21 |
| SKT-M-26 | | |
| SL-34.0829 | | WO 0209761 A2 |
| SL 651708 | | |
| SL 851016 | | |
| SL-870495 | | |
| SM-6586 | | EP 00177965 |
| SNX-124 | | |
| SNX 185 | w-Conotoxin G | WO 9310145 A1 |
| | VIA | |
| SNX-236 | | WO 09313128 |
| SNX-239 | | Pain (1995), 60(1), 83-90 |
| SNX-482, peptides from tarantula | | WO 9805780 A2 |
| venom | | |
| sornidipine | | |
| SQ 31486 | | EP 205334 A2 |
| SQ-31727 | | |
| SQ 31765 | | |
| SQ 32321 | | |
| SQ 32324 | | |
| SQ 32547 | | EP 400665 A2 |
| SQ 32926 | | EP 400665 A2 |
| SQ-33351 | | WO 09006118 |
| SQ 33537 | | |
| SQ-34399 | | |
| SR-33805 | | EP 576347 A1 |
| SUN 5647 | | |
| SUN 6087 | | |
| SUN-N8075 | | WO 9923072 A1 |
| T-477 | | EP 00441539 |
| TA-993 | | JP 01050872 |
| taludipine | | |
| tamolarizine | | EP 00354068 |
| TDN-345 | | |
| Teczem | | |
| temiverine | Urespan, NS 21 | CAN 131: 193592 |
| terflavoxate | | EP 72620 A1 |
| terodiline TD 758 | Bicor | U.S. Pat. No. 3371014 |
| tetrandrine | | Clinical and Experimental |
| | | Pharmacology and |
| | | Physiology (1996), 23(8), |
| | | 751-753 |
| TH-1177 | | |
| TH-9229 | | WO 09607415 |
| thapsigargin | | British Journal of |
| | | Pharmacology (1985 Jul), |
| | | 85(3), 705-12 |
| tiapamil | | |
| tinctormine | | Chemical & Pharmaceutical |
| | | Bulletin (1992), 40 (12), |
| | | 3355-7 |
| TJN 220 O-Ethylfangchinoline | | JP 63179878 A2 |
| TMB 8 | | Journal of Cell Science |
| | | (1985 Nov), 79 151-60 |
| TN-871 | | European Journal of |
| | | Pharmacology (1998), |
| | | 342(2/3), 167-175 |
| TR 2957 | | |
| trapidil | | |
| trimetazidine | Adexor, Idaptan, | U.S. Pat. No. 3262852 |
| | Preductal, | |
| | S 5016, | |
| | Trimetazine, | |
| | Vastarel | |
| TY-10835 | | Pharmacometrics |
| | | 1998, 54: 3 (153) |
| U-88999 | | WO 9204338 |
| U-92032 | | WO 09204338 |
| U-92798 | | WO 9204338 A1 |
| UK 1745 | | EP 653426 A1 |
| UK-51656 | | EP 00089167 |
| UK 52831 | | JP 59118782 A2 |
| UK-55444 | | EP 00132375 |
| UK 56593 | | |
| UK-84149 | | EP 404359 A1 |
| ULAH 99 | | European Journal of |
| | | Pharmacology (1992 Dec. |
| | | 8), 229(1), 55-62 |
| vantanipidine | Calbren | EP 257616 A2 |
| verapamil, verelan | Calan, Covera, | U.S. Pat. No. 3261859 |
| | Isopin Verelan | |
| S-verapamil D-2024 | | WO 09509150 |
| levoverapamil | | |
| vexibinol Sophoraflavanone G | | Chemical and |
| | | Pharmaceutical Bulletin |
| | | (1990 Apr), 38(4), 1039-44 |
| vinigrol | | |
| vintoperol RGH 2981 RT 303 | | WO 9207851 |
| VUF-8929 | | EP 467435 A2 |
| VULM 993 | | |
| vantanipidine | Calbren | EP 257616 A2 |
| W 787 | | |
| WAS 4206 | | |
| WK 269 | | |
| WY 27569 | | |
| WY 44644 | | |
| WY 44705 | | |
| WY 46622 | | |
| WY 47324 | | |
| xanthonolol | | U.S. Pat. No. 5495005 |
| Y 19638 | | |
| Y-22516 | | WO 9323082 |
| Y 208835 | | |
| YC 114 | | |
| YH-334 | | EP 00366548 |
| YM 15430-1 (see YM 430) | | |
| YM-16151-4 (YM 151) | | EP 00167371 |
| YM-430 (YM 15430) | | WO 0209761 A2 |
| YS 035 | | BE 897244 A2 |
| YS 161 | | |
| Z-6568 | | Journal of Mass |
| | | Spectrometry (1996), |
| | | 31(1), 37-46 |
| ziconotide omega connotoxin | Prialt, CI 1009, | WO 9107980 |
| MVIIA SNX-111 | SNX 194 | |
| ZM-224832 | | EP 00343865 |
| zonisamide | Excegran, | U.S. Pat. No. 4172896 |
| | Zonegran | |

Generally speaking, the pharmacokinetics of the particular agent to be administered will dictate the most preferred method of administration and dosing regiment. The calcium modulating agent can be administered as a pharmaceutical composition with or without a carrier. The terms “pharmaceutically acceptable carrier” or a “carrier” refer to any generally acceptable excipient or drug delivery composition that is relatively inert and non-toxic. Exemplary carriers include sterile water, salt solutions (such as Ringer's solution), alcohols, gelatin, talc, viscous paraffin, fatty acid esters, hydroxymethylcellulose, polyvinyl pyrolidone, calcium carbonate, carbohydrates (such as lactose, sucrose, dextrose, mannose, albumin, starch, cellulose, silica gel, polyethylene glycol (PEG), dried skim milk, rice flour, magnesium stearate, and the like. Suitable formulations and additional carriers are described in Remington's Pharmaceutical Sciences, (17.sup.th Ed., Mack Pub. Co., Easton, Pa.). Such preparations can be sterilized and, if desired, mixed with auxiliary agents, e.g., lubricants, preservatives, stabilizers, wetting agents, emulsifiers, salts for influencing osmotic pressure, buffers, coloring, preservatives and/or aromatic substances and the like which do not deleteriously react with the active compounds. Typical preservatives can include, potassium sorbate, sodium metabisulfite, methyl paraben, propyl paraben, thimerosal, etc. The compositions can also be combined where desired with other active substances, e.g., enzyme inhibitors, to reduce metabolic degradation.

Moreover, the calcium modulating agent can be a liquid solution, suspension, emulsion, tablet, pill, capsule, sustained release formulation, or powder. The method of administration can dictate how the composition will be formulated. For example, the composition can be formulated as a suppository, with traditional binders and carriers such as triglycerides. Oral formulation can include standard carriers such as pharmaceutical grades of mannitol, lactose, starch, magnesium stearate, sodium saccharine, cellulose, or magnesium carbonate.

In another embodiment, the calcium modulating agent can be administered intravenously, parenterally, intramuscular, subcutaneously, orally, nasally, topically, by inhalation, by implant, by injection, or by suppository. For enteral or mucosal application (including via oral and nasal mucosa), particularly suitable are tablets, liquids, drops, suppositories or capsules. A syrup, elixir or the like can be used wherein a sweetened vehicle is employed. Liposomes, microspheres, and microcapsules are available and can be used. Pulmonary administration can be accomplished, for example, using any of various delivery devices known in the art such as an inhaler. See. e.g. S. P. Newman (1984) in Aerosols and the Lung, Clarke and Davis (eds.), Butterworths, London, England, pp. 197-224; PCT Publication No. WO 92/16192; PCT Publication No. WO 91/08760. For parenteral application, particularly suitable are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-polyoxypropylene block polymers, and the like.

The actual effective amounts of compound or drug can and will vary according to the specific composition being utilized, the mode of administration and the age, weight and condition of the subject. By way of example, as used herein, an effective amount of the calcium modulating agent is an amount that achieves the desired degree of inhibition of Ca²⁺ ion flow down the electrochemical gradient of one or more calcium channels. Dosages for a particular individual subject can be determined by one of ordinary skill in the art using conventional considerations. But in general, the amount of calcium modulating agent will be between about 10 to about 2500 milligrams per day. The daily dose can be administered in one to four doses per day.

In one embodiment, when the calcium modulating agent comprises nimodipine, typically the amount administered is within a range of from about 0.5 to about 500 milligrams per day, and even more typically, between about 40 to about 240 milligrams per day.

In another embodiment, when the calcium modulating agent is flunarizine, typically the amount administered is within a range of from about 0.5 to about 100 milligrams per day, and even more typically, between about 1 to about 10 milligrams per day.

In yet another embodiment, when the calcium modulating agent is bepridil, generally the amount administered is within a range of from about 0.5 to about 500 milligrams per day, and even more typically, between about 200 to about 400 milligrams per day.

In still another embodiment, when the calcium modulating agent is diltiazem, typically the amount administered is within a range of from about 0.5 to about 50 milligrams per hour, and even more typically, between about 5 to about 15 milligrams per hour.

In yet a further embodiment, when the calcium modulating agent is felodipine, typically the amount administered is within a range of from about 0.5 to about 50 milligrams per day, and even more typically, between about 5 to about 20 milligrams per day.

In still another embodiment, when the calcium modulating agent is isradipine, typically the amount administered is within a range of from about 0.5 to about 50 milligrams per day, and even more typically, between about 2.5 to about 20 milligrams per day.

In yet another embodiment, when the calcium modulating agent is nicardipine, typically the amount administered is within a range of from about 0.5 to about 100 milligrams per hour, and even more typically, between about 20 to about 40 milligrams per hour.

In yet a further embodiment, when the calcium modulating agent is nifedipine, typically the amount administered is within a range of from about 0.5 to about 500 milligrams per day, and even more typically, between about 30 to about 120 milligrams per day.

In still another embodiment, when the calcium modulating agent is verapamil, typically the amount administered is within a range of from about 0.5 to about 1000 milligrams per day, and even more typically, between about 180 to about 540 milligrams per day.

In one embodiment, when the calcium modulating agent comprises lacidipine, typically the amount administered is within a range of from about 0.5 to about 100 milligrams per day, and even more typically, between about 1 to about 10 milligrams per day.

In another embodiment, when the calcium modulating agent is lomerizine, typically the amount administered is within a range of from about 0.5 to about 100 milligrams per day, and even more typically, between about 1 to about 20 milligrams per day.

In yet another embodiment, when the calcium modulating agent is propiverine, generally the amount administered is within a range of from about 0.5 to about 100 milligrams per day, and even more typically, between about 15 to about 60 milligrams per day.

In still another embodiment, when the calcium modulating agent is trimetazidine, typically the amount administered is within a range of from about 0.5 to about 100 milligrams per day, and even more typically, between about 20 to about 60 milligrams per day.

In yet a further embodiment, when the calcium modulating agent is zonisamide, typically the amount administered is within a range of from about 0.5 to about 1000 milligrams per day, and even more typically, between about 100 to about 600 milligrams per day.

In still another embodiment, when the calcium modulating agent is lercanidipine, typically the amount administered is within a range of from about 0.5 to about 100 milligrams per day, and even more typically, between about 10 to about 20 milligrams per day.

In still another embodiment, when the calcium modulating agent is nilvadipine, typically the amount administered is within a range of from about 0.5 to about 50 milligrams per hour, and even more typically, between about 4 to about 16 milligrams per hour.

In yet a further embodiment, when the calcium modulating agent is benidipine, typically the amount administered is within a range of from about 0.5 to about 100 milligrams per day, and even more typically, between about 2 to about 20 milligrams per day.

In still another embodiment, when the calcium modulating agent is nisoldipine, typically the amount administered is within a range of from about 0.5 to about 100 milligrams per day, and even more typically, between about 10 to about 20 milligrams per day.

In one embodiment, when the calcium modulating agent comprises nitrendipine, typically the amount administered is within a range of from about 0.5 to about 100 milligrams per day, and even more typically, between about 10 to about 40 milligrams per day.

In another embodiment, when the calcium modulating agent is manidipine, typically the amount administered is within a range of from about 0.5 to about 100 milligrams per day, and even more typically, between about 10 to about 20 milligrams per day.

In yet another embodiment, when the calcium modulating agent is barnidipine, generally the amount administered is within a range of from about 0.5 to about 100 milligrams per day, and even more typically, between about 10 to about 30 milligrams per day.

In still another embodiment, when the calcium modulating agent is efonidipine, typically the amount administered is within a range of from about 0.5 to about 100 milligrams per day, and even more typically, between about 20 to about 40 milligrams per day.

In yet a further embodiment, when the calcium modulating agent is amlodipine, typically the amount administered is within a range of from about 0.5 to about 50 milligrams per day, and even more typically, between about 5 to about 10 milligrams per day.

In still another embodiment, when the calcium modulating agent is cilnidipine, typically the amount administered is within a range of from about 0.5 to about 50 milligrams per day, and even more typically, between about 5 to about 20 milligrams per day.

In still another embodiment, when the calcium modulating agent is lercanidipine, typically the amount administered is within a range of from about 0.5 to about 100 milligrams per hour, and even more typically, between about 10 to about 30 milligrams per hour.

In yet a further embodiment, when the calcium modulating agent is aranidipine, typically the amount administered is within a range of from about 0.5 to about 100 milligrams per day, and even more typically, between about 1.25 to about 20 milligrams per day.

In yet a further embodiment, when the calcium modulating agent is mibefradil, typically the amount administered is within a range of from about 0.5 to about 500 milligrams per day, and even more typically, between about 10 to about 100 milligrams per day.

Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

The timing of the administration of the cyclooxygenase-2 selective inhibitor in relation to the administration of the calcium modulating agent may also vary from subject to subject. In one embodiment, the cyclooxygenase-2 selective inhibitor and calcium modulating agent may be administered substantially simultaneously, meaning that both agents may be administered to the subject at approximately the same time. For example, the cyclooxygenase-2 selective is administered during a continuous period beginning on the same day as the beginning of the calcium modulating agent and extending to a period after the end of the calcium modulating agent. Alternatively, the cyclooxygenase-2 selective inhibitor and calcium modulating agent may be administered sequentially, meaning that they are administered at separate times during separate treatments. In one embodiment, for example, the cyclooxygenase-2 selective inhibitor is administered during a continuous period beginning prior to administration of the calcium modulating agent and ending after administration of the calcium modulating agent. Of course, it is also possible that the cyclooxygenase-2 selective inhibitor may be administered either more or less frequently than the calcium modulating agent. Moreover, it will be apparent to those skilled in the art that it is possible, and perhaps desirable, to combine various times and methods of administration in the practice of the present invention.

Combination Therapies

Generally speaking, it is contemplated that the composition employed in the practice of the invention may include one or more of any of the cyclooxygenase-2 selective inhibitors detailed above in combination with one or more of any of the calcium modulating agents detailed above. By way of a non-limiting example, Table 9a details a number of suitable combinations that are useful in the methods and compositions of the current invention. The combination may also include an isomer, a pharmaceutically acceptable salt, ester, or prodrug of any of the cyclooxygenase-2 selective inhibitors or calcium modulating agents listed in Table 9a.

TABLE 9a

| | Calcium |
|---|---|
| Cyclooxygenase-2 Selective Inhibitor | Modulating Agent |
| a compound having formula I | nimodipine |
| a compound having formula I | nicardipine |
| a compound having formula I | nifedipine |
| a compound having formula I | amolodipine |
| a compound having formula I | isradipine |
| a compound having formula I | diltiazem |
| a compound having formula I | verapamil |
| a compound having formula I | bepridil |
| a compound having formula I | gallopamil |
| a compound having formula I | flunarizine |
| a compound having formula I | pimozide |
| a compound having formula II | nimodipine |
| a compound having formula II | nicardipine |
| a compound having formula II | nifedipine |
| a compound having formula II | amolodipine |
| a compound having formula II | isradipine |
| a compound having formula II | diltiazem |
| a compound having formula II | verapamil |
| a compound having formula II | bepridil |
| a compound having formula II | gallopamil |
| a compound having formula II | flunarizine |
| a compound having formula II | pimozide |
| a compound having formula III | nimodipine |
| a compound having formula III | nicardipine |
| a compound having formula III | nifedipine |
| a compound having formula III | amolodipine |
| a compound having formula III | isradipine |
| a compound having formula III | diltiazem |
| a compound having formula III | verapamil |
| a compound having formula III | bepridil |
| a compound having formula III | gallopamil |
| a compound having formula III | flunarizine |
| a compound having formula III | pimozide |
| a compound having formula IV | nimodipine |
| a compound having formula IV | nicardipine |
| a compound having formula IV | nifedipine |
| a compound having formula IV | amolodipine |
| a compound having formula IV | isradipine |
| a compound having formula IV | diltiazem |
| a compound having formula IV | verapamil |
| a compound having formula IV | bepridil |
| a compound having formula IV | gallopamil |
| a compound having formula IV | flunarizine |
| a compound having formula IV | pimozide |
| a compound having formula V | nimodipine |
| a compound having formula V | nicardipine |
| a compound having formula V | nifedipine |
| a compound having formula V | amolodipine |
| a compound having formula V | isradipine |
| a compound having formula V | diltiazem |
| a compound having formula V | verapamil |
| a compound having formula V | bepridil |
| a compound having formula V | gallopamil |
| a compound having formula V | flunarizine |
| a compound having formula V | pimozide |

By way of further example, Table 9b details a number of suitable combinations that may be employed in the methods and compositions of the present invention. The combination may also include an isomer, a pharmaceutically acceptable salt, ester, or prodrug of any of the cyclooxygenase-2 selective inhibitors or calcium modulating agents listed in Table 9b.

TABLE 9b

| | Calcium |
|---|---|
| | Modulating |
| Cyclooxygenase-2 Selective Inhibitor | Agent |
| a compound selected from the group consisting of B-1, | nimodipine |
| B-2, B-3, B-4, B-5, B-6, B-7, B-8, B-9, B-10, B-11, | |
| B-12, B-13, B-14, B-15, B-16, B-17, B-18, B-19, | |
| B-20, B-21, B-22, B-23, B-24, B-25, B-26, B-27, | |
| B-28, B-29, B-30, B-31, B-32, B-33, B-34, B-35, B-36, | |
| B-37, B-38, B-39, B-40, B-41, B-42, B-43, B-44, | |
| B-45, B-46, B-47, B-48, B-49, B-50, B-51, B-52, | |
| B-53, B-54, B-55, B-56, B-57, B-58, B-59, B-60, | |
| B-61, B-62, B-63, B-64, B-65, B-66, B-67, B-68, | |
| B-69, B-70, B-71, B-72, B-73, B-74, B-75, B-76, | |
| B-77, B-78, B-79, B-80, B-81, B-82, B-83, B-84, | |
| B-85, B-86, B-87, B-88, B-89, B-90, B-91, B-92, | |
| B-93, B-94, B-95, B-96, B-97, B-98, B-99, | |
| B-100, B-101, B-102, B-103, B-104, B-105, B-106, | |
| B-107, B-108, B-109, B-110, B-111, B-112, B-113, | |
| B-114, B-115, B-116, B-117, B-118, B-119, B-120, | |
| B-121, B-122, B-123, B-124, B-125, B-126, B-127, | |
| B-128, B-129, B-130, B-131, B-132, B-133, B-134, | |
| B-135, B-136, B-137, B-138, B-139, B-140, B-141, | |
| B-142, B-143, B-144, B-145, B-146, B-147, B-148, | |
| B-149, B-150, B-151, B-152, B-153, B-154, B-155, | |
| B-156, B-157, B-158, B-159, B-160, B-161, B-162, | |
| B-163, B-164, B-165, B-166, B-167, B-165, B-169, | |
| B-170, B-171, B-172, B-173, B-174, B-175, B-176, | |
| B-177, B-178, B-179, B-180, B-181, B-182, B-183, | |
| B-184, B-185, B-186, B-187, B-188, B-189, B-190, | |
| B-191, B-192, B-193, B-194, B-195, B-196, B-197, | |
| B-198, B-199, B-200, B-201, B-202, B-203, B-204, | |
| B-205, B-206, B-207, B-208, B-209, B-210, B-211, | |
| B-212, B-213, B-214, B-215, B-216, B-217, B-218, | |
| B-219, B-220, B-221, B-222, B-223, B-224, B-225, | |
| B-226, B-227, B-228, B-229, B-230, B-231, B-232, | |
| B233, B-234, B-235, B-236, B-237, B-238, B-239, | |
| B-240, B-241, B-242, B-243 B-244, B-245, B-246, | |
| B-247, B-248, B-249, B-250, B-251, B-252 | |
| a compound selected from the group consisting of B-1, | nicardipine |
| B-2, B-3, B-4, B-5, B-6, B-7, B-8, B-9, B-10, B-11, | |
| B-12, B-13, B-14, B-15, B-16, B-17, B-18, B-19, | |
| B-20, B-21, B-22, B-23, B-24, B-25, B-26, B-27, | |
| B-28, B-29, B-30, B-31, B-32, B-33, B-34, B-35, B-36, | |
| B-37, B-38, B-39, B-40, B-41, B-42, B-43, B-44, | |
| B-45, B-46, B-47, B-48, B-49, B-50, B-51, B-52, | |
| B-53, B-54, B-55, B-56, B-57, B-58, B-59, B-60, | |
| B-61, B-62, B-63, B-64, B-65, B-66, B-67, B-68, | |
| B-69, B-70, B-71, B-72, B-73, B-74, B-75, B-76, | |
| B-77, B-78, B-79, B-80, B-81, B-82, B-83, B-84, | |
| B-85, B-86, B-87, B-88, B-89, B-90, B-91, B-92, | |
| B-93, B-94, B-95, B-96, B-97, B-98, B-99, | |
| B-100, B-101, B-102, B-103, B-104, B-105, B-106, | |
| B-107, B-108, B-109, B-110, B-111, B-112, B-113, | |
| B-114, B-115, B-116, B-117, B-118, B-119, B-120, | |
| B-121, B-122, B-123, B-124, B-125, B-126, B-127, | |
| B-128, B-129, B-130, B-131, B-132, B-133, B-134, | |
| B-135, B-136, B-137, B-138, B-139, B-140, B-141, | |
| B-142, B-143, B-144, B-145, B-146, B-147, B-148, | |
| B-149, B-150, B-151, B-152, B-153, B-154, B-155, | |
| B-156, B-157, B-158, B-159, B-160, B-161, B-162, | |
| B-163, B-164, B-165, B-166, B-167, B-168, B-169, | |
| B-170, B-171, B-172, B-173, B-174, B-175, B-176, | |
| B-177, B-178, B-179, B-180, B-181, B-182, B-183, | |
| B-184, B-185, B-186, B-187, B-188, B-189, B-190, | |
| B-191, B-192, B-193, B-194, B-195, B-196, B-197, | |
| B-198, B-199, B-200, B-201, B-202, B-203, B-204, | |
| B-205, B-206, B-207, B-208, B-209, B-210, B-211, | |
| B-212, B-213, B-214, B-215, B-216, B-217, B-218, | |
| B-219, B-220, B-221, B-222, B-223, B-224, B-225, | |
| B-226, B-227, B-228, B-229, B-230, B-231, B-232, | |
| B233, B-234, B-235, B-236, B-237, B-238, B-239, | |
| B-240, B-241, B-242, B-243 B-244, B-245, B-246, | |
| B-247, B-248, B-249, B-250, B-251, B-252 | |
| a compound selected from the group consisting of B-1, | nifedipine |
| B-2, B-3, B-4, B-5, B-6, B-7, B-8, B-9, B-10, B-11, | |
| B-12, B-13, B-14, B-15, B-16, B-17, B-18, B-19, | |
| B-20, B-21, B-22, B-23, B-24, B-25, B-26, B-27, | |
| B-28, B-29, B-30, B-31, B-32, B-33, B-34, B-35, B-36, | |
| B-37, B-38, B-39, B-40, B-41, B-42, B-43, B-44, | |
| B-45, B-46, B-47, B-48, B-49, B-50, B-51, B-52, | |
| B-53, B-54, B-55, B-56, B-57, B-58, B-59, B-60, | |
| B-61, B-62, B-63, B-64, B-65, B-66, B-67, B-68, | |
| B-69, B-70, B-71, B-72, B-73, B-74, B-75, B-76, | |
| B-77, B-78, B-79, B-80, B-81, B-82, B-83, B-84, | |
| B-85, B-86, B-87, B-88, B-89, B-90, B-91, B-92, | |
| B-93, B-94, B-95, B-96, B-97, B-98, B-99, | |
| B-100, B-101, B-102, B-103, B-104, B-105, B-106, | |
| B-107, B-108, B-109, B-110, B-111, B-112, B-113, | |
| B-114, B-115, B-116, B-117, B-118, B-119, B-120, | |
| B-121, B-122, B-123, B-124, B-125, B-126, B-127, | |
| B-128, B-129, B-130, B-131, B-132, B-133, B-134, | |
| B-135, B-136, B-137, B-138, B-139, B-140, B-141, | |
| B-142, B-143, B-144, B-145, B-146, B-147, B-148, | |
| B-149, B-150, B-151, B-152, B-153, B-154, B-155, | |
| B-156, B-157, B-158, B-159, B-160, B-161, B-162, | |
| B-163, B-164, B-165, B-166, B-167, B-168, B-169, | |
| B-170, B-171, B-172, B-173, B-174, B-175, B-176, | |
| B-177, B-178, B-179, B-180, B-181, B-182, B-183, | |
| B-184, B-185, B-186, B-187, B-188, B-189, B-190, | |
| B-191, B-192, B-193, B-194, B-195, B-196, B-197, | |
| B-198, B-199, B-200, B-201, B-202, B-203, B-204, | |
| B-205, B-206, B-207, B-208, B-209, B-210, B-211, | |
| B-212, B-213, B-214, B-215, B-216, B-217, B-218, | |
| B-219, B-220, B-221, B-222, B-223, B-224, B-225, | |
| B-226, B-227, B-228, B-229, B-230, B-231, B-232, | |
| B233, B-234, B-235, B-236, B-237, B-238, B-239, | |
| B-240, B-241, B-242, B-243 B-244, B-245, B-246, | |
| B-247, B-248, B-249, B-250, B-251, B-252 | |
| a compound selected from the group consisting of B-1, | amolodipine |
| B-2, B-3, B-4, B-5, B-6, B-7, B-8, B-9, B-10, B-11, | |
| B-12, B-13, B-14, B-15, B-16, B-17, B-18, B-19, | |
| B-20, B-21, B-22, B-23, B-24, B-25, B-26, B-27, | |
| B-28, B-29, B-30, B-31, B-32, B-33, B-34, B-35, B-36, | |
| B-37, B-38, B-39, B-40, B-41, B-42, B-43, B-44, | |
| B-45, B-46, B-47, B-48, B-49, B-50, B-51, B-52, | |
| B-53, B-54, B-55, B-56, B-57, B-58, B-59, B-60, | |
| B-61, B-62, B-63, B-64, B-65, B-66, B-67, B-68, | |
| B-69, B-70, B-71, B-72, B-73, B-74, B-75, B-76, | |
| B-77, B-78, B-79, B-80, B-81, B-82, B-83, B-84, | |
| B-85, B-86, B-87, B-88, B-89, B-90, B-91, B-92, | |
| B-93, B-94, B-95, B-96, B-97, B-98, B-99, | |
| B-100, B-101, B-102, B-103, B-104, B-105, B-106, | |
| B-107, B-108, B-109, B-110, B-111, B-112, B-113, | |
| B-114, B-115, B-116, B-117, B-118, B-119, B-120, | |
| B-121, B-122, B-123, B-124, B-125, B-126, B-127, | |
| B-128, B-129, B-130, B-131, B-132, B-133, B-134, | |
| B-135, B-136, B-137, B-138, B-139, B-140, B-141, | |
| B-142, B-143, B-144, B-145, B-146, B-147, B-148, | |
| B-149, B-150, B-151, B-152, B-153, B-154, B-155, | |
| B-156, B-157, B-158, B-159, B-160, B-161, B-162, | |
| B-163, B-164, B-165, B-166, B-167, B-168, B-169, | |
| B-170, B-171, B-172, B-173, B-174, B-175, B-176, | |
| B-177, B-178, B-179, B-180, B-181, B-182, B-183, | |
| B-184, B-185, B-186, B-187, B-188, B-189, B-190, | |
| B-191, B-192, B-193, B-194, B-195, B-196, B-197, | |
| B-198, B-199, B-200, B-201, B-202, B-203, B-204, | |
| B-205, B-206, B-207, B-208, B-209, B-210, B-211, | |
| B-212, B-213, B-214, B-215, B-216, B-217, B-218, | |
| B-219, B-220, B-221, B-222, B-223, B-224, B-225, | |
| B-226, B-227, B-228, B-229, B-230, B-231, B-232, | |
| B233, B-234, B-235, B-236, B-237, B-238, B-239, | |
| B-240, B-241, B-242, B-243 B-244, B-245, B-246, | |
| B-247, B-248, B-249, B-250, B-251, B-252 | |
| a compound selected from the group consisting of B-1, | isradipine |
| B-2, B-3, B-4, B-5, B-6, B-7, B-8, B-9, B-10, B-11, | |
| B-12, B-13, B-14, B-15, B-16, B-17, B-18, B-19, | |
| B-20, B-21, B-22, B-23, B-24, B-25, B-26, B-27, | |
| B-28, B-29, B-30, B-31, B-32, B-33, B-34, B-35, B-36, | |
| B-37, B-38, B-39, B-40, B-41, B-42, B-43, B-44, | |
| B-45, B-46, B-47, B-48, B-49, B-50, B-51, B-52, | |
| B-53, B-54, B-55, B-56, B-57, B-58, B-59, B-60, | |
| B-61, B-62, B-63, B-64, B-65, B-66, B-67, B-68, | |
| B-69, B-70, B-71, B-72, B-73, B-74, B-75, B-76, | |
| B-77, B-78, B-79, B-80, B-81, B-82, B-83, B-84, | |
| B-85, B-86, B-87, B-88, B-89, B-90, B-91, B-92, | |
| B-93, B-94, B-95, B-96, B-97, B-98, B-99, | |
| B-100, B-101, B-102, B-103, B-104, B-105, B-106, | |
| B-107, B-108, B-109, B-110, B-111, B-112, B-113, | |
| B-114, B-115, B-116, B-117, B-118, B-119, B-120, | |
| B-121, B-122, B-123, B-124, B-125, B-126, B-127, | |
| B-128, B-129, B-130, B-131, B-132, B-133, B-134, | |
| B-135, B-136, B-137, B-138, B-139, B-140, B-141, | |
| B-142, B-143, B-144, B-145, B-146, B-147, B-148, | |
| B-149, B-150, B-151, B-152, B-153, B-154, B-155, | |
| B-156, B-157, B-158, B-159, B-160, B-161, B-162, | |
| B-163, B-164, B-165, B-166, B-167, B-168, B-169, | |
| B-170, B-171, B-172, B-173, B-174, B-175, B-176, | |
| B-177, B-178, B-179, B-180, B-181, B-182, B-183, | |
| B-184, B-185, B-186, B-187, B-188, B-189, B-190, | |
| B-191, B-192, B-193, B-194, B-195, B-196, B-197, | |
| B-198, B-199, B-200, B-201, B-202, B-203, B-204, | |
| B-205, B-206, B-207, B-208, B-209, B-210, B-211, | |
| B-212, B-213, B-214, B-215, B-216, B-217, B-218, | |
| B-219, B-220, B-221, B-222, B-223, B-224, B-225, | |
| B-226, B-227, B-228, B-229, B-230, B-231, B-232, | |
| B233, B-234, B-235, B-236, B-237, B-238, B-239, | |
| B-240, B-241, B-242, B-243 B-244, B-245, B-246, | |
| B-247, B-248, B-249, B-250, B-251, B-252 | |
| a compound selected from the group consisting of B-1, | diltiazem |
| B-2, B-3, B-4, B-5, B-6, B-7, B-8, B-9, B-10, B-11, | |
| B-12, B-13, B-14, B-15, B-16, B-17, B-18, B-19, | |
| B-20, B-21, B-22, B-23, B-24, B-25, B-26, B-27, | |
| B-28, B-29, B-30, B-31, B-32, B-33, B-34, B-35, B-36, | |
| B-37, B-38, B-39, B-40, B-41, B-42, B-43, B-44, | |
| B-45, B-46, B-47, B-48, B-49, B-50, B-51, B-52, | |
| B-53, B-54, B-55, B-56, B-57, B-58, B-59, B-60, | |
| B-61, B-62, B-63, B-64, B-65, B-66, B-67, B-68, | |
| B-69, B-70, B-71, B-72, B-73, B-74, B-75, B-76, | |
| B-77, B-78, B-79, B-80, B-81, B-82, B-83, B-84, | |
| B-85, B-86, B-87, B-88, B-89, B-90, B-91, B-92, | |
| B-93, B-94, B-95, B-96, B-97, B-98, B-99, | |
| B-100, B-101, B-102, B-103, B-104, B-105, B-106, | |
| B-107, B-108, B-109, B-110, B-111, B-112, B-113, | |
| B-114, B-115, B-116, B-117, B-118, B-119, B-120, | |
| B-121, B-122, B-123, B-124, B-125, B-126, B-127, | |
| B-128, B-129, B-130, B-131, B-132, B-133, B-134, | |
| B-135, B-136, B-137, B-138, B-139, B-140, B-141, | |
| B-142, B-143, B-144, B-145, B-146, B-147, B-148, | |
| B-149, B-150, B-151, B-152, B-153, B-154, B-155, | |
| B-156, B-157, B-158, B-159, B-160, B-161, B-162, | |
| B-163, B-164, B-165, B-166, B-167, B-168, B-169, | |
| B-170, B-171, B-172, B-173, B-174, B-175, B-176, | |
| B-177, B-178, B-179, B-180, B-181, B-182, B-183, | |
| B-184, B-185, B-186, B-187, B-188, B-189, B-190, | |
| B-191, B-192, B-193, B-194, B-195, B-196, B-197, | |
| B-198, B-199, B-200, B-201, B-202, B-203, B-204, | |
| B-205, B-206, B-207, B-208, B-209, B-210, B-211, | |
| B-212, B-213, B-214, B-215, B-216, B-217, B-218, | |
| B-219, B-220, B-221, B-222, B-223, B-224, B-225, | |
| B-226, B-227, B-228, B-229, B-230, B-231, B-232, | |
| B233, B-234, B-235, B-236, B-237, B-238, B-239, | |
| B-240, B-241, B-242, B-243 B-244, B-245, B-246, | |
| B-247, B-248, B-249, B-250, B-251, B-252 | |
| a compound selected from the group consisting of B-1, | verapamil |
| B-2, B-3, B-4, B-5, B-6, B-7, B-8, B-9, B-10, B-11, | |
| B-12, B-13, B-14, B-15, B-16, B-17, B-18, B-19, | |
| B-20, B-21, B-22, B-23, B-24, B-25, B-26, B-27, | |
| B-28, B-29, B-30, B-31, B-32, B-33, B-34, B-35, B-36, | |
| B-37, B-38, B-39, B-40, B-41, B-42, B-43, B-44, | |
| B-45, B-46, B-47, B-48, B-49, B-50, B-51, B-52, | |
| B-53, B-54, B-55, B-56, B-57, B-58, B-59, B-60, | |
| B-61, B-62, B-63, B-64, B-65, B-66, B-67, B-68, | |
| B-69, B-70, B-71, B-72, B-73, B-74, B-75, B-76, | |
| B-77, B-78, B-79, B-80, B-81, B-82, B-83, B-84, | |
| B-85, B-86, B-87, B-88, B-89, B-90, B-91, B-92, | |
| B-93, B-94, B-95, B-96, B-97, B-98, B-99, | |
| B-100, B-101, B-102, B-103, B-104, B-105, B-106, | |
| B-107, B-108, B-109, B-110, B-111, B-112, B-113, | |
| B-114, B-115, B-116, B-117, B-118, B-119, B-120, | |
| B-121, B-122, B-123, B-124, B-125, B-126, B-127, | |
| B-128, B-129, B-130, B-131, B-132, B-133, B-134, | |
| B-135, B-136, B-137, B-138, B-139, B-140, B-141, | |
| B-142, B-143, B-144, B-145, B-146, B-147, B-148, | |
| B-149, B-150, B-151, B-152, B-153, B-154, B-155, | |
| B-156, B-157, B-158, B-159, B-160, B-161, B-162, | |
| B-163, B-164, B-165, B-166, B-167, B-168, B-169, | |
| B-170, B-171, B-172, B-173, B-174, B-175, B-176, | |
| B-177, B-178, B-179, B-180, B-181, B-182, B-183, | |
| B-184, B-185, B-186, B-187, B-188, B-189, B-190, | |
| B-191, B-192, B-193, B-194, B-195, B-196, B-197, | |
| B-198, B-199, B-200, B-201, B-202, B-203, B-204, | |
| B-205, B-206, B-207, B-208, B-209, B-210, B-211, | |
| B-212, B-213, B-214, B-215, B-216, B-217, B-218, | |
| B-219, B-220, B-221, B-222, B-223, B-224, B-225, | |
| B-226, B-227, B-228, B-229, B-230, B-231, B-232, | |
| B233, B-234, B-235, B-236, B-237, B-238, B-239, | |
| B-240, B-241, B-242, B-243 B-244, B-245, B-246, | |
| B-247, B-248, B-249, B-250, B-251, B-252 | |
| a compound selected from the group consisting of B-1, | bepridil |
| B-2, B-3, B-4, B-5, B-6, B-7, B-8, B-9, B-10, B-11, | |
| B-12, B-13, B-14, B-15, B-16, B-17, B-18, B-19, | |
| B-20, B-21, B-22, B-23, B-24, B-25, B-26, B-27, | |
| B-28, B-29, B-30, B-31, B-32, B-33, B-34, B-35, B-36, | |
| B-37, B-38, B-39, B-40, B-41, B-42, B-43, B-44, | |
| B-45, B-46, B-47, B-48, B-49, B-50, B-51, B-52, | |
| B-53, B-54, B-55, B-56, B-57, B-58, B-59, B-60, | |
| B-61, B-62, B-63, B-64, B-65, B-66, B-67, B-68, | |
| B-69, B-70, B-71, B-72, B-73, B-74, B-75, B-76, | |
| B-77, B-78, B-79, B-80, B-81, B-82, B-83, B-84, | |
| B-85, B-86, B-87, B-88, B-89, B-90, B-91, B-92, | |
| B-93, B-94, B-95, B-96, B-97, B-98, B-99, | |
| B-100, B-101, B-102, B-103, B-104, B-105, B-106, | |
| B-107, B-108, B-109, B-110, B-111, B-112, B-113, | |
| B-114, B-115, B-116, B-117, B-118, B-119, B-120, | |
| B-121, B-122, B-123, B-124, B-125, B-126, B-127, | |
| B-128, B-129, B-130, B-131, B-132, B-133, B-134, | |
| B-135, B-136, B-137, B-138, B-139, B-140, B-141, | |
| B-142, B-143, B-144, B-145, B-146, B-147, B-148, | |
| B-149, B-150, B-151, B-152, B-153, B-154, B-155, | |
| B-156, B-157, B-158, B-159, B-160, B-161, B-162, | |
| B-163, B-164, B-165, B-166, B-167, B-168, B-169, | |
| B-170, B-171, B-172, B-173, B-174, B-175, B-176, | |
| B-177, B-178, B-179, B-180, B-181, B-182, B-183, | |
| B-184, B-185, B-186, B-187, B-188, B-189, B-190, | |
| B-191, B-192, B-193, B-194, B-195, B-196, B-197, | |
| B-198, B-199, B-200, B-201, B-202, B-203, B-204, | |
| B-205, B-206, B-207, B-208, B-209, B-210, B-211, | |
| B-212, B-213, B-214, B-215, B-216, B-217, B-218, | |
| B-219, B-220, B-221, B-222, B-223, B-224, B-225, | |
| B-226, B-227, B-228, B-229, B-230, B-231, B-232, | |
| B233, B-234, B-235, B-236, B-237, B-238, B-239, | |
| B-240, B-241, B-242, B-243 B-244, B-245, B-246, | |
| B-247, B-248, B-249, B-250, B-251, B-252 | |
| a compound selected from the group consisting of B-1, | gallopamil |
| B-2, B-3, B-4, B-5, B-6, B-7, B-8, B-9, B-10, B-11, | |
| B-12, B-13, B-14, B-15, B-16, B-17, B-18, B-19, | |
| B-20, B-21, B-22, B-23, B-24, B-25, B-26, B-27, | |
| B-28, B-29, B-30, B-31, B-32, B-33, B-34, B-35, B-36, | |
| B-37, B-38, B-39, B-40, B-41, B-42, B-43, B-44, | |
| B-45, B-46, B-47, B-48, B-49, B-50, B-51, B-52, | |
| B-53, B-54, B-55, B-56, B-57, B-58, B-59, B-60, | |
| B-61, B-62, B-63, B-64, B-65, B-66, B-67, B-68, | |
| B-69, B-70, B-71, B-72, B-73, B-74, B-75, B-76, | |
| B-77, B-78, B-79, B-80, B-81, B-82, B-83, B-84, | |
| B-85, B-86, B-87, B-88, B-89, B-90, B-91, B-92, | |
| B-93, B-94, B-95, B-96, B-97, B-98, B-99, | |
| B-100, B-101, B-102, B-103, B-104, B-105, B-106, | |
| B-107, B-108, B-109, B-110, B-111, B-112, B-113, | |
| B-114, B-115, B-116, B-117, B-118, B-119, B-120, | |
| B-121, B-122, B-123, B-124, B-125, B-126, B-127, | |
| B-128, B-129, B-130, B-131, B-132, B-133, B-134, | |
| B-135, B-136, B-137, B-138, B-139, B-140, B-141, | |
| B-142, B-143, B-144, B-145, B-146, B-147, B-148, | |
| B-149, B-150, B-151, B-152, B-153, B-154, B-155, | |
| B-156, B-157, B-158, B-159, B-160, B-161, B-162, | |
| B-163, B-164, B-165, B-166, B-167, B-168, B-169, | |
| B-170, B-171, B-172, B-173, B-174, B-175, B-176, | |
| B-177, B-178, B-179, B-180, B-181, B-182, B-183, | |
| B-184, B-185, B-186, B-187, B-188, B-189, B-190, | |
| B-191, B-192, B-193, B-194, B-195, B-196, B-197, | |
| B-198, B-199, B-200, B-201, B-202, B-203, B-204, | |
| B-205, B-206, B-207, B-208, B-209, B-210, B-211, | |
| B-212, B-213, B-214, B-215, B-216, B-217, B-218, | |
| B-219, B-220, B-221, B-222, B-223, B-224, B-225, | |
| B-226, B-227, B-228, B-229, B-230, B-231, B-232, | |
| B233, B-234, B-235, B-236, B-237, B-238, B-239, | |
| B-240, B-241, B-242, B-243 B-244, B-245, B-246, | |
| B-247, B-248, B-249, B-250, B-251, B-252 | |
| a compound selected from the group consisting of B-1, | flunarizine |
| B-2, B-3, B-4, B-5, B-6, B-7, B-8, B-9, B-10, B-11, | |
| B-12, B-13, B-14, B-15, B-16, B-17, B-18, B-19, | |
| B-20, B-21, B-22, B-23, B-24, B-25, B-26, B-27, | |
| B-28, B-29, B-30, B-31, B-32, B-33, B-34, B-35, B-36, | |
| B-37, B-38, B-39, B-40, B-41, B-42, B-43, B-44, | |
| B-45, B-46, B-47, B-48, B-49, B-50, B-51, B-52, | |
| B-53, B-54, B-55, B-56, B-57, B-58, B-59, B-60, | |
| B-61, B-62, B-63, B-64, B-65, B-66, B-67, B-68, | |
| B-69, B-70, B-71, B-72, B-73, B-74, B-75, B-76, | |
| B-77, B-78, B-79, B-80, B-81, B-82, B-83, B-84, | |
| B-85, B-86, B-87, B-88, B-89, B-90, B-91, B-92, | |
| B-93, B-94, B-95, B-96, B-97, B-98, B-99, | |
| B-100, B-101, B-102, B-103, B-104, B-105, B-106, | |
| B-107, B-108, B-109, B-110, B-111, B-112, B-113, | |
| B-114, B-115, B-116, B-117, B-118, B-119, B-120, | |
| B-121, B-122, B-123, B-124, B-125, B-126, B-127, | |
| B-128, B-129, B-130, B-131, B-132, B-133, B-134, | |
| B-135, B-136, B-137, B-138, B-139, B-140, B-141, | |
| B-142, B-143, B-144, B-145, B-146, B-147, B-148, | |
| B-149, B-150, B-151, B-152, B-153, B-154, B-155, | |
| B-156, B-157, B-158, B-159, B-160, B-161, B-162, | |
| B-163, B-164, B-165, B-166, B-167, B-168, B-169, | |
| B-170, B-171, B-172, B-173, B-174, B-175, B-176, | |
| B-177, B-178, B-179, B-180, B-181, B-182, B-183, | |
| B-184, B-185, B-186, B-187, B-188, B-189, B-190, | |
| B-191, B-192, B-193, B-194, B-195, B-196, B-197, | |
| B-198, B-199, B-200, B-201, B-202, B-203, B-204, | |
| B-205, B-206, B-207, B-208, B-209, B-210, B-211, | |
| B-212, B-213, B-214, B-215, B-216, B-217, B-218, | |
| B-219, B-220, B-221, B-222, B-223, B-224, B-225, | |
| B-226, B-227, B-228, B-229, B-230, B-231, B-232, | |
| B233, B-234, B-235, B-236, B-237, B-238, B-239, | |
| B-240, B-241, B-242, B-243 B-244, B-245, B-246, | |
| B-247, B-248, B-249, B-250, B-251, B-252 | |
| a compound selected from the group consisting of B-1, | pimozide |
| B-2, B-3, B-4, B-5, B-6, B-7, B-8, B-9, B-10, B-11, | |
| B-12, B-13, B-14, B-15, B-16, B-17, B-18, B-19, | |
| B-20, B-21, B-22, B-23, B-24, B-25, B-26, B-27, | |
| B-28, B-29, B-30, B-31, B-32, B-33, B-34, B-35, B-36, | |
| B-37, B-38, B-39, B-40, B-41, B-42, B-43, B-44, | |
| B-45, B-46, B-47, B-48, B-49, B-50, B-51, B-52, | |
| B-53, B-54, B-55, B-56, B-57, B-58, B-59, B-60, | |
| B-61, B-62, B-63, B-64, B-65, B-66, B-67, B-68, | |
| B-69, B-70, B-71, B-72, B-73, B-74, B-75, B-76, | |
| B-77, B-78, B-79, B-80, B-81, B-82, B-83, B-84, | |
| B-85, B-86, B-87, B-88, B-89, B-90, B-91, B-92, | |
| B-93, B-94, B-95, B-96, B-97, B-98, B-99, | |
| B-100, B-101, B-102, B-103, B-104, B-105, B-106, | |
| B-107, B-108, B-109, B-110, B-111, B-112, B-113, | |
| B-114, B-115, B-116, B-117, B-118, B-119, B-120, | |
| B-121, B-122, B-123, B-124, B-125, B-126, B-127, | |
| B-128, B-129, B-130, B-131, B-132, B-133, B-134, | |
| B-135, B-136, B-137, B-138, B-139, B-140, B-141, | |
| B-142, B-143, B-144, B-145, B-146, B-147, B-148, | |
| B-149, B-150, B-151, B-152, B-153, B-154, B-155, | |
| B-156, B-157, B-158, B-159, B-160, B-161, B-162, | |
| B-163, B-164, B-165, B-166, B-167, B-168, B-169, | |
| B-170, B-171, B-172, B-173, B-174, B-175, B-176, | |
| B-177, B-178, B-179, B-180, B-181, B-182, B-183, | |
| B-184, B-185, B-186, B-187, B-188, B-189, B-190, | |
| B-191, B-192, B-193, B-194, B-195, B-196, B-197, | |
| B-198, B-199, B-200, B-201, B-202, B-203, B-204, | |
| B-205, B-206, B-207, B-208, B-209, B-210, B-211, | |
| B-212, B-213, B-214, B-215, B-216, B-217, B-218, | |
| B-219, B-220, B-221, B-222, B-223, B-224, B-225, | |
| B-226, B-227, B-228, B-229, B-230, B-231, B-232, | |
| B233, B-234, B-235, B-236, B-237, B-238, B-239, | |
| B-240, B-241, B-242, B-243 B-244, B-245, B-246, | |
| B-247, B-248, B-249, B-250, B-251, B-252 | |

By way of yet further example, Table 9c details additional suitable combinations that may be employed in the methods and compositions of the current invention. The combination may also include an isomer, a pharmaceutically acceptable salt, ester, or prodrug of any of the cyclooxygenase-2 selective inhibitors or calcium modulating agents listed in Table 9c.

TABLE 9c

| Cyclooxygenase-2 Selective Inhibitor | Calcium Modulating Agent |
|---|---|
| Celecoxib | nimodipine |
| Celecoxib | nicardipine |
| Celecoxib | nifedipine |
| Celecoxib | amolodipine |
| Celecoxib | isradipine |
| Celecoxib | diltiazem |
| Celecoxib | verapamil |
| Celecoxib | bepridil |
| Celecoxib | gallopamil |
| Celecoxib | flunarizine |
| Celecoxib | pimozide |
| Deracoxib | nimodipine |
| Deracoxib | nicardipine |
| Deracoxib | nifedipine |
| Deracoxib | amolodipine |
| Deracoxib | isradipine |
| Deracoxib | diltiazem |
| Deracoxib | verapamil |
| Deracoxib | bepridil |
| Deracoxib | gallopamil |
| Deracoxib | flunarizine |
| Deracoxib | pimozide |
| Valdecoxib | nimodipine |
| Valdecoxib | nicardipine |
| Valdecoxib | nifedipine |
| Valdecoxib | amolodipine |
| Valdecoxib | isradipine |
| Valdecoxib | diltiazem |
| Valdecoxib | verapamil |
| Valdecoxib | bepridil |
| Valdecoxib | gallopamil |
| Valdecoxib | flunarizine |
| Valdecoxib | pimozide |
| Rofecoxib | nimodipine |
| Rofecoxib | nicardipine |
| Rofecoxib | nifedipine |
| Rofecoxib | amolodipine |
| Rofecoxib | isradipine |
| Rofecoxib | diltiazem |
| Rofecoxib | verapamil |
| Rofecoxib | bepridil |
| Rofecoxib | gallopamil |
| Rofecoxib | flunarizine |
| Rofecoxib | pimozide |
| Etoricoxib | nimodipine |
| Etoricoxib | nicardipine |
| Etoricoxib | nifedipine |
| Etoricoxib | amolodipine |
| Etoricoxib | isradipine |
| Etoricoxib | diltiazem |
| Etoricoxib | verapamil |
| Etoricoxib | bepridil |
| Etoricoxib | gallopamil |
| Etoricoxib | flunarizine |
| Etoricoxib | pimozide |
| meloxicam | nimodipine |
| meloxicam | nicardipine |
| meloxicam | nifedipine |
| meloxicam | amolodipine |
| meloxicam | isradipine |
| meloxicam | diltiazem |
| meloxicam | verapamil |
| meloxicam | bepridil |
| meloxicam | gallopamil |
| meloxicam | flunarizine |
| meloxicam | pimozide |
| Parecoxib | nimodipine |
| Parecoxib | nicardipine |
| Parecoxib | nifedipine |
| Parecoxib | amolodipine |
| Parecoxib | isradipine |
| Parecoxib | diltiazem |
| Parecoxib | verapamil |
| Parecoxib | bepridil |
| Parecoxib | gallopamil |
| Parecoxib | flunarizine |
| Parecoxib | pimozide |
| 4-(4-cyclohexyl-2-methyloxazol-5-yl)-2- | nimodipine |
| fluorobenzenesulfonamide | |
| 4-(4-cyclohexyl-2-methyloxazol-5-yl)-2- | nicardipine |
| fluorobenzenesulfonamide | |
| 4-(4-cyclohexyl-2-methyloxazol-5-yl)-2- | nifedipine |
| fluorobenzenesulfonamide | |
| 4-(4-cyclohexyl-2-methyloxazol-5-yl)-2- | amolodipine |
| fluorobenzenesulfonamide | |
| 4-(4-cyclohexyl-2-methyloxazol-5-yl)-2- | isradipine |
| fluorobenzenesulfonamide | |
| 4-(4-cyclohexyl-2-methyloxazol-5-yl)-2- | diltiazem |
| fluorobenzenesulfonamide | |
| 4-(4-cyclohexyl-2-methyloxazol-5-yl)-2- | verapamil |
| fluorobenzenesulfonamide | |
| 4-(4-cyclohexyl-2-methyloxazol-5-yl)-2- | bepridil |
| fluorobenzenesulfonamide | |
| 4-(4-cyclohexyl-2-methyloxazol-5-yl)-2- | gallopamil |
| fluorobenzenesulfonamide | |
| 4-(4-cyclohexyl-2-methyloxazol-5-yl)-2- | flunarizine |
| fluorobenzenesulfonamide | |
| 4-(4-cyclohexyl-2-methyloxazol-5-yl)-2- | pimozide |
| fluorobenzenesulfonamide | |
| 2-(3,5-difluorophenyl)-3-(4- | nimodipine |
| (methylsulfonyl)phenyl)-2-cyclopenten-1-one | |
| 2-(3,5-difluorophenyl)-3-(4- | nicardipine |
| (methylsulfonyl)phenyl)-2-cyclopenten-1-one | |
| 2-(3,5-difluorophenyl)-3-(4- | nifedipine |
| (methylsulfonyl)phenyl)-2-cyclopenten-1-one | |
| 2-(3,5-difluorophenyl)-3-(4- | amolodipine |
| (methylsulfonyl)phenyl)-2-cyclopenten-1-one | |
| 2-(3,5-difluorophenyl)-3-(4- | isradipine |
| (methylsulfonyl)phenyl)-2-cyclopenten-1-one | |
| 2-(3,5-difluorophenyl)-3-(4- | diltiazem |
| (methylsulfonyl)phenyl)-2-cyclopenten-1-one | |
| 2-(3,5-difluorophenyl)-3-(4- | verapamil |
| (methylsulfonyl)phenyl)-2-cyclopenten-1-one | |
| 2-(3,5-difluorophenyl)-3-(4- | bepridil |
| (methylsulfonyl)phenyl)-2-cyclopenten-1-one | |
| 2-(3,5-difluorophenyl)-3-(4- | gallopamil |
| (methylsulfonyl)phenyl)-2-cyclopenten-1-one | |
| 2-(3,5-difluorophenyl)-3-(4- | flunarizine |
| (methylsulfonyl)phenyl)-2-cyclopenten-1-one | |
| 2-(3,5-difluorophenyl)-3-(4- | pimozide |
| (methylsulfonyl)phenyl)-2-cyclopenten-1-one | |
| N-[2-(cyclohexyloxy)-4- | nimodipine |
| nitrophenyl]methanesulfonamide | |
| N-[2-(cyclohexyloxy)-4- | nicardipine |
| nitrophenyl]methanesulfonamide | |
| N-[2-(cyclohexyloxy)-4- | nifedipine |
| nitrophenyl]methanesulfonamide | |
| N-[2-(cyclohexyloxy)-4- | amolodipine |
| nitrophenyl]methanesulfonamide | |
| N-[2-(cyclohexyloxy)-4- | isradipine |
| nitrophenyl]methanesulfonamide | |
| N-[2-(cyclohexyloxy)-4- | diltiazem |
| nitrophenyl]methanesulfonamide | |
| N-[2-(cyclohexyloxy)-4- | verapamil |
| nitrophenyl]methanesulfonamide | |
| N-[2-(cyclohexyloxy)-4- | bepridil |
| nitrophenyl]methanesulfonamide | |
| N-[2-(cyclohexyloxy)-4- | gallopamil |
| nitrophenyl]methanesulfonamide | |
| N-[2-(cyclohexyloxy)-4- | flunarizine |
| nitrophenyl]methanesulfonamide | |
| N-[2-(cyclohexyloxy)-4- | pimozide |
| nitrophenyl]methanesulfonamide | |
| 2-(3,4-difluorophenyl)-4-(3-hydroxy-3- | nimodipine |
| methylbutoxy)-5-[4-(methylsulfonyl)phenyl]- | |
| 3(2H)-pyridazinone | |
| 2-(3,4-difluorophenyl)-4-(3-hydroxy-3- | nicardipine |
| methylbutoxy)-5-[4-(methylsulfonyl)phenyl]- | |
| 3(2H)-pyridazinone | |
| 2-(3,4-difluorophenyl)-4-(3-hydroxy-3- | nifedipine |
| methylbutoxy)-5-[4-(methylsulfonyl)phenyl]- | |
| 3(2H)-pyridazinone | |
| 2-(3,4-difluorophenyl)-4-(3-hydroxy-3- | amolodipine |
| methylbutoxy)-5-[4-(methylsulfonyl)phenyl]- | |
| 3(2H)-pyridazinone | |
| 2-(3,4-difluorophenyl)-4-(3-hydroxy-3- | isradipine |
| methylbutoxy)-5-[4-(methylsulfonyl)phenyl]- | |
| 3(2H)-pyridazinone | |
| 2-(3,4-difluorophenyl)-4-(3-hydroxy-3- | diltiazem |
| methylbutoxy)-5-[4-(methylsulfonyl)phenyl]- | |
| 3(2H)-pyridazinone | |
| 2-(3,4-difluorophenyl)-4-(3-hydroxy-3- | verapamil |
| methylbutoxy)-5-[4-(methylsulfonyl)phenyl]- | |
| 3(2H)-pyridazinone | |
| 2-(3,4-difluorophenyl)-4-(3-hydroxy-3- | bepridil |
| methylbutoxy)-5-[4-(methylsulfonyl)phenyl]- | |
| 3(2H)-pyridazinone | |
| 2-(3,4-difluorophenyl)-4-(3-hydroxy-3- | gallopamil |
| methylbutoxy)-5-[4-(methylsulfonyl)phenyl]- | |
| 3(2H)-pyridazinone | |
| 2-(3,4-difluorophenyl)-4-(3-hydroxy-3- | flunarizine |
| methylbutoxy)-5-[4-(methylsulfonyl)phenyl]- | |
| 3(2H)-pyridazinone | |
| 2-(3,4-difluorophenyl)-4-(3-hydroxy-3- | pimozide |
| methylbutoxy)-5-[4-(methylsulfonyl)phenyl]- | |
| 3(2H)-pyridazinone | |
| 2-[(2,4-dichloro-6-methylphenyl)amino]-5- | nimodipine |
| ethyl-benzeneacetic acid | |
| 2-[(2,4-dichloro-6-methylphenyl)amino]-5- | nicardipine |
| ethyl-benzeneacetic acid | |
| 2-[(2,4-dichloro-6-methylphenyl)amino]-5- | nifedipine |
| ethyl-benzeneacetic acid | |
| 2-[(2,4-dichloro-6-methylphenyl)amino]-5- | amolodipine |
| ethyl-benzeneacetic acid | |
| 2-[(2,4-dichloro-6-methylphenyl)amino]-5- | isradipine |
| ethyl-benzeneacetic acid | |
| 2-[(2,4-dichloro-6-methylphenyl)amino]-5- | diltiazem |
| ethyl-benzeneacetic acid | |
| 2-[(2,4-dichloro-6-methylphenyl)amino]-5- | verapamil |
| ethyl-benzeneacetic acid | |
| 2-[(2,4-dichloro-6-methylphenyl)amino]-5- | bepridil |
| ethyl-benzeneacetic acid | |
| 2-[(2,4-dichloro-6-methylphenyl)amino]-5- | gallopamil |
| ethyl-benzeneacetic acid | |
| 2-[(2,4-dichloro-6-methylphenyl)amino]-5- | flunarizine |
| ethyl-benzeneacetic acid | |
| 2-[(2,4-dichloro-6-methylphenyl)amino]-5- | pimozide |
| ethyl-benzeneacetic acid | |
| (3Z)-3-[(4-chlorophenyl)[4- | nimodipine |
| (methylsulfonyl)phenyl]methylene]dihydro- | |
| 2(3H)-furanone | |
| (3Z)-3-[(4-chlorophenyl)[4- | nicardipine |
| (methylsulfonyl)phenyl]methylene]dihydro- | |
| 2(3H)-furanone | |
| (3Z)-3-[(4-chlorophenyl)[4- | nifedipine |
| (methylsulfonyl)phenyl]methylene]dihydro- | |
| 2(3H)-furanone | |
| (3Z)-3-[(4-chlorophenyl)[4- | amolodipine |
| (methylsulfonyl)phenyl]methylene]dihydro- | |
| 2(3H)-furanone | |
| (3Z)-3-[(4-chlorophenyl)[4- | isradipine |
| (methylsulfonyl)phenyl]methylene]dihydro- | |
| 2(3H)-furanone | |
| (3Z)-3-[(4-chlorophenyl)[4- | diltiazem |
| (methylsulfonyl)phenyl]methylene]dihydro- | |
| 2(3H)-furanone | |
| (3Z)-3-[(4-chlorophenyl)[4- | verapamil |
| (methylsulfonyl)phenyl]methylene]dihydro- | |
| 2(3H)-furanone | |
| (3Z)-3-[(4-chlorophenyl)[4- | bepridil |
| (methylsulfonyl)phenyl]methylene]dihydro- | |
| 2(3H)-furanone | |
| (3Z)-3-[(4-chlorophenyl)[4- | gallopamil |
| (methylsulfonyl)phenyl]methylene]dihydro- | |
| 2(3H)-furanone | |
| (3Z)-3-[(4-chlorophenyl)[4- | flunarizine |
| (methylsulfonyl)phenyl]methylene]dihydro- | |
| 2(3H)-furanone | |
| (3Z)-3-[(4-chlorophenyl)[4- | pimozide |
| (methylsulfonyl)phenyl]methylene]dihydro- | |
| 2(3H)-furanone | |
| (S)-6,8-dichloro-2-(trifluoromethyl)-2H-1- | nimodipine |
| benzopyran-3-carboxylic acid | |
| (S)-6,8-dichloro-2-(trifluoromethyl)-2H-1- | nicardipine |
| benzopyran-3-carboxylic acid | |
| (S)-6,8-dichloro-2-(trifluoromethyl)-2H-1- | nifedipine |
| benzopyran-3-carboxylic acid | |
| (S)-6,8-dichloro-2-(trifluoromethyl)-2H-1- | amolodipine |
| benzopyran-3-carboxylic acid | |
| (S)-6,8-dichloro-2-(trifluoromethyl)-2H-1- | isradipine |
| benzopyran-3-carboxylic acid | |
| (S)-6,8-dichloro-2-(trifluoromethyl)-2H-1- | diltiazem |
| benzopyran-3-carboxylic acid | |
| (S)-6,8-dichloro-2-(trifluoromethyl)-2H-1- | verapamil |
| benzopyran-3-carboxylic acid | |
| (S)-6,8-dichloro-2-(trifluoromethyl)-2H-1- | bepridil |
| benzopyran-3-carboxylic acid | |
| (S)-6,8-dichloro-2-(trifluoromethyl)-2H-1- | gallopamil |
| benzopyran-3-carboxylic acid | |
| (S)-6,8-dichloro-2-(trifluoromethyl)-2H-1- | flunarizine |
| benzopyran-3-carboxylic acid | |
| (S)-6,8-dichloro-2-(trifluoromethyl)-2H-1- | pimozide |
| benzopyran-3-carboxylic acid | |
| lumiracoxib | nimodipine |
| lumiracoxib | nicardipine |
| lumiracoxib | nifedipine |
| lumiracoxib | amolodipine |
| Lumiracoxib | isradipine |
| Lumiracoxib | diltiazem |
| Lumiracoxib | verapamil |
| Lumiracoxib | bepridil |
| Lumiracoxib | gallopamil |
| Lumiracoxib | flunarizine |
| Lumiracoxib | pimozide |

Indication to be Treated

Generally speaking, the composition comprising a therapeutically effective amount of a cyclooxygenase-2 selective inhibitor and a therapeutically effective amount of a calcium modulating agent may be employed for symptomatic treatment of pain sensation and to treat inflammation, and inflammation mediated disorder.

One aspect of the invention encompasses administering the composition to a subject for symptomatic treatment of neuropathic pain. Neuropathic pain is pain that is due to functional abnormalities of the nervous system. In general, there are a variety of possible mechanisms by which nerve dysfunction can cause neuropathic pain: hyperactivity in primary afferent or central nervous system nociceptive neurons, loss of central inhibitory connections, and increased activity in sympathetic efferents. The composition of the invention may be utilized to treat neuropathic pain irrespective of the underlying mechanism causing the pain. Examples of causes of painful nerve injury that may be treated by the composition of the invention include accidental trauma, tumors, cerval or lumbar spine disease, and surgical procedures. Additionally, there are also toxic, metabolic, and hereditary causes of painful polyneuropathies, e.g., alcohol abuse, diabetes mellitus that may be treated by the composition of the invention.

In an alternative of this embodiment, the composition may be employed to treat allodynia and hyperalgesia neuropathic pain. Generally speaking, allodynia and hyperalgesia describes a particular type of pain sensation that differs from the customary perception of painful stimuli. Subjects who suffer from hyperalgesic pain feel painful stimuli more strongly than healthy subjects do. Alternatively, subjects who suffer from allodynia perceive stimuli that are not painful per se, such as contact or heat/cold, as pain.

Another aspect of the invention encompasses administering the composition to a subject for symptomatic treatment of nociceptive pain. Nociceptive pain includes all forms of somatic pain that result from damage or dysfunction of non-neural tissue. The composition may be employed to treat either acute or chronic nociceptive pain. Typically, acute nociceptive pain includes pain resulting from tissue-damaging stimulation such as that produced by injury or disease. Examples include postoperative pain, post traumatic pain, acute pancreatis, labor pain, muscle pain and pain accompanying myocardial infarction. Chronic nociceptive pain typically lasts for a longer duration of time relative to the duration of acute pain. Examples of chronic pain that may be treated by the composition include inflammatory pain; arthritis pain, cancer pain and other forms of persistent pain deriving from damaged or inflamed somatic tissue.

Yet another aspect of the invention encompasses administering the composition to lessen symptomatic pain resulting from a number of different disorders or disease states. In one embodiment, the composition may be administered to treat long-lasting allodynia resulting from herpes zoster (shingles) infection. In another embodiment, the composition may be administered to an AIDS patient, to treat pain in various stages of the disorder. In yet another embodiment, the composition may be administered to a subject with cancer to relieve pain resulting from either the cancer itself or for pain resulting from the treatment of cancer. By way of example, therapy with high doses of cytostatics for cancer generally causes pain. By way of further example, a tumor disorder itself can also elicit neuropathic pain that may be treated by the composition of the invention. In still another embodiment, a subject with chronic back pain, such as resulting from a compression of nerve roots of the spinal cord, can be treated by the composition of the invention. In yet another embodiment, a subject with a spinal cord injury, which often results in very severe pain sensations, may be treated by the composition of the invention.

A further aspect of the invention comprises administering the composition to treat inflammation or inflammation mediated disorders, such as those mediated by cyclooxygenase-2. Typical conditions benefited by cyclooxygenase-2 selective inhibition include the treatment or prevention of inflammation, and for treatment or prevention of other inflammation-associated disorders, such as, an analgesic in the treatment of pain and headaches, or as an antipyretic for the treatment of fever. For example, the composition is useful to treat or prevent arthritis, including but not limited to rheumatoid arthritis, spondyloarthopathies, gouty arthritis, osteoarthritis, systemic lupus erythematosus and juvenile arthritis. The composition is also useful in the treatment or prevention of asthma, bronchitis, menstrual cramps, tendinitis, bursitis, skin-related conditions such as psoriasis, eczema, burns and dermatitis, and from post-operative inflammation including ophthalmic surgery such as cataract surgery and refractive surgery. Moreover, the composition may be employed to treat or prevent gastrointestinal conditions such as inflammatory bowel disease, Crohn's disease, gastritis, irritable bowel syndrome and ulcerative colitis. The composition may also be employed in treating or preventing inflammation in such diseases as vascular diseases, migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury, myocardial ischemia, and the like.

### EXAMPLES

A combination therapy of a COX-2 selective inhibitor and a calcium modulating agent for the treatment of pain, inflammation or inflammation mediated disorders in a subject can be evaluated as described in the following tests detailed below.

A particular combination therapy comprising a calcium modulating agent and a COX-2 inhibitor can be evaluated in comparison to a control treatment such as a placebo treatment, administration of a COX-2 inhibitor-only, or administration of a calcium modulating agent only. By way of example, a combination therapy may contain any of the calcium modulating agents and COX-2 inhibitors detailed in the present invention, including the combinations set forth in Tables 9a, 9b, or 9c may be tested as a combination therapy. The dosages of a calcium modulating agent and COX-2 inhibitor in a particular therapeutic combination may be readily determined by a skilled artisan conducting the study. The length of the study treatment will vary on a particular study and can also be determined by one of ordinary skill in the art. By way of example, the combination therapy may be administered for 4 weeks. The calcium modulating agent and COX-2 inhibitor can be administered by any route as described herein, but are preferably administered orally for human subjects.

### Example 1

### Evaluation of COX-1 and COX-2 Activity In Vitro

The COX-2 inhibitors suitable for use in this invention exhibit selective inhibition of COX-2 over COX-1 when tested in vitro according to the following activity assays.

Preparation of Recombinant COX Baculoviruses

Recombinant COX-1 and COX-2 are prepared as described by Gierse et al, [J. Biochem., 305, 479-84 (1995)]. A 2.0 kb fragment containing the coding region of either human or murine COX-1 or human or murine COX-2 is cloned into a BamH1 site of the baculovirus transfer vector pVL1393 (Invitrogen) to generate the baculovirus transfer vectors for COX-1 and COX-2 in a manner similar to the method of D. R. O'Reilly et al (Baculovirus Expression Vectors: A Laboratory Manual (1992)). Recombinant baculoviruses are isolated by transfecting 4 μg of baculovirus transfer vector DNA into SF9 insect cells (2×10⁸) along with 200 ng of linearized baculovirus plasmid DNA by the calcium phosphate method. See M. D. Summers and G. E. Smith, A Manual of Methods for Baculovirus Vectors and Insect Cell Culture Procedures, Texas Agric. Exp. Station Bull. 1555 (1987). Recombinant viruses are purified by three rounds of plaque purification and high titer (10⁷-10⁸ pfu/mL) stocks of virus are prepared. For large scale production, SF9 insect cells are infected in 10 liter fermentors (0.5×106/mL) with the recombinant baculovirus stock such that the multiplicity of infection is 0.1. After 72 hours the cells are centrifuged and the cell pellet is homogenized in Tris/Sucrose (50 mM: 25%, pH 8.0) containing 1% 3-[(3-cholamidopropyl)-dimethylammonio]-1-propanesulfonate (CHAPS). The homogenate is centrifuged at 10,000×G for 30 minutes, and the resultant supernatant is stored at −80° C. before being assayed for COX activity.

Assay for COX-1 and COX-2 Activity

COX activity is assayed as PGE2 formed/μg protein/time using an ELISA to detect the prostaglandin released. CHAPS-solubilized insect cell membranes containing the appropriate COX enzyme are incubated in a potassium phosphate buffer (50 mM, pH 8.0) containing epinephrine, phenol, and heme with the addition of arachidonic acid (10 μM). Compounds are pre-incubated with the enzyme for 10-20 minutes prior to the addition of arachidonic acid. Any reaction between the arachidonic acid and the enzyme is stopped after ten minutes at 37° C. by transferring 40 μl of reaction mix into 160 μl ELISA buffer and 25 μM indomethacin. The PGE2 formed is measured by standard ELISA technology (Cayman Chemical).

Fast Assay for COX-1 and COX-2 Activity

COX activity is assayed as PGE2 formed/μg protein/time using an ELISA to detect the prostaglandin released. CHAPS-solubilized insect cell membranes containing the appropriate COX enzyme are incubated in a potassium phosphate buffer (0.05 M Potassium phosphate, pH 7.5, 2 μM phenol, 1 μM heme, 300 μM epinephrine) with the addition of 20 μl of 100 μM arachidonic acid (10 μM). Compounds are pre-incubated with the enzyme for 10 minutes at 25° C. prior to the addition of arachidonic acid. Any reaction between the arachidonic acid and the enzyme is stopped after two minutes at 37° C. by transferring 40 μl of reaction mix into 160 μl ELISA buffer and 25 μM indomethacin. Indomethacin, a non-selective COX-2/COX-1 inhibitor, may be utilized as a positive control. The PGE₂ formed is typically measured by standard ELISA technology utilizing a PGE2 specific antibody, available from a number of commercial sources.

Each compound to be tested may be individually dissolved in 2 ml of dimethyl sulfoxide (DMSO) for bioassay testing to determine the COX-1 and COX-2 inhibitory effects of each particular compound. Potency is typically expressed by the IC₅₀ value expressed as g compound/ml solvent resulting in a 50% inhibition of PGE2 production. Selective inhibition of COX-2 may be determined by the IC₅₀ ratio of COX-1/COX-2.

By way of example, a primary screen may be performed in order to determine particular compounds that inhibit COX-2 at a concentration of 10 ug/ml. The compound may then be subjected to a confirmation assay to determine the extent of COX-2 inhibition at three different concentrations (e.g., 10 ug/ml, 3.3 ug/ml and 1.1 ug/ml). After this screen, compounds can then be tested for their ability to inhibit COX-1 at a concentration of 10 ug/ml. With this assay, the percentage of COX inhibition compared to control can be determined, with a higher percentage indicating a greater degree of COX inhibition. In addition, the IC₅₀ value for COX-1 and COX-2 can also be determined for the tested compound. The selectivity for each compound may then be determined by the IC₅₀ ratio of COX-1/COX-2, as set-forth above.

### Example 2

### Rat Carrageenan Foot Pad Edema Test

The anti-inflammatory properties of COX-2 selective inhibitors for use, along with their combination with a calcium modulating agent, in the present methods can be determined by the rat carrageenan footpad edema test. The carrageenan foot edema test is performed with materials, reagents and procedures essentially as described by Winter, et al., (Proc. Soc. Exp. Biol. Med., 111: 544, 1962). Male Sprague-Dawley rats are selected in each group so that the average body weight is as close as possible. Rats are fasted with free access to water for over sixteen hours prior to the test. The rats are dosed orally (1 mL) with compounds suspended in vehicle containing 0.5% methylcellulose and 0.025% surfactant, or with vehicle alone. One hour later, a subplantar injection of 0.1 mL of 1% solution of carrageenan/sterile 0.9% saline is administered and the volume of the injected foot is measured with a displacement plethysmometer connected to a pressure transducer with a digital indicator. Three hours after the injection of the carrageenan, the volume of the foot is again measured. The average foot swelling in a group of drug-treated animals is compared with that of a group of placebo-treated animals and the percentage inhibition of edema is determined (Otterness and Bliven, Laboratory Models for Testing NSAIDs, in Non-steroidal Anti-Inflammatory Drugs, (J. Lombardino, ed. 1985)). The percentage inhibition shows the percentage decrease from control paw volume determined in this procedure.

Rats may be administered any COX-2 inhibitor and any calcium modulating agent described herein. By way of example, the COX-2 inhibitor may be selected from the group consisting of celecoxib, rofecoxib, valdecoxib, etoricoxib, parecoxib, and deracoxib and the calcium modulating agent may be selected from the group consisting of gallopamil, bepridil, mibefradil, nickel chloride, ethosuximide, pimozide, ziconotide, bepridil, verapamil, nimodipine, nicardipine, nifedipine, amolodipine and isradipine.

### Example 3

### Rat Plantar Test

The ability of COX-2 selective inhibitors along with a calcium modulating agent for use in the method of the present invention to prevent hyperalgesia can be determined by the rat plantar test. The rat plantar test is performed with materials, reagents and procedures essentially as described by Hargreaves et al. (Pain. (1988) 32:77-88). Male Sprague-Dawley rats are selected in each group so that the average body weight is as close as possible. An inflammation is induced in the rats by intraplantar injection of an approximately 0.05% suspension of Mycobacterium butyricum. Six hours after this injection, a heat stimulus is applied by infrared ray onto the plantar face of the hind paw of the rat. The nociceptive reaction of the rat manifests itself by the withdrawal or the licking of the paw. The time of this pain reaction is then measured. Additionally the COX-2 selective inhibitor and calcium modulating agent are administered via the oral route approximately one hour before the plantar test. The average time of pain reaction in a group of drug-treated animals is then compared with that of a group of placebo-treated animals in order to determine the hyperalgesia preventative effect of the composition of the present invention.

Rats may be administered any COX-2 inhibitor and any calcium modulating agent described herein. By way of example, the COX-2 inhibitor may be selected from the group consisting of celecoxib, rofecoxib, valdecoxib, etoricoxib, parecoxib, and deracoxib and the calcium modulating agent may be selected from the group consisting of gallopamil, bepridil, mibefradil, nickel chloride, ethosuximide, pimozide, ziconotide, bepridil, verapamil, nimodipine, nicardipine, nifedipine, amolodipine and isradipine.

### Example 4

### Phenylbenzoquinone Test

The analgesic properties of COX-2 selective inhibitors along with a calcium modulating agent for use in the present methods can be determined by the phenylbenzoquinone test. The phenylbenzoquinone test is performed with the materials, reagents, and procedures essentially as described in Siegmund et al. (Proc. Sec. Exp. Biol. Med. (1957) 95:729-731). Male Sprague-Dawley rats are selected in each group so that the average body weight is as close as possible. One hour after the oral administration of the composition of the present invention, a 0.02% solution of phenylbenzoquinone is administered via the intra-peritoneal route to each rat. The number of pain reactions, measured as abdominal torsions and stretches, is then counted between the fifth and sixth minute after injection of the phenylbenzoquinone. The average number of pain reactions in a group of drug-treated animals is then compared with that of a group of placebo-treated animals in order to determine the analgesic properties of the composition of the present invention.

Rats may be administered any COX-2 inhibitor and any calcium modulating agent described herein. By way of example, the COX-2 inhibitor may be selected from the group consisting of celecoxib, rofecoxib, valdecoxib, etoricoxib, parecoxib, and deracoxib and the calcium modulating agent may be selected from the group consisting of gallopamil, bepridil, mibefradil, nickel chloride, ethosuximide, pimozide, ziconotide, bepridil, verapamil, nimodipine, nicardipine, nifedipine, amolodipine and isradipine.

What is claimed is:

1. A method of treating pain, inflammation or an inflammation mediated disorder, the method comprising:
(a) diagnosing a subject in need of treatment for pain, inflammation or an inflammation mediated disorder; and
(b) administering to the subject a cyclooxygenase-2 selective inhibitor or an isomer, a pharmaceutically acceptable salt, ester, or prodrug thereof and a calcium modulating agent or an isomer, a pharmaceutically acceptable salt, ester, or prodrug thereof.

2. The method of claim 1 wherein the cyclooxgenase-2 selective inhibitor has a selectivity ratio of COX-1 IC₅₀ to COX-2 IC₅₀ not less than about 50.

3. The method of claim 1 wherein the cyclooxgenase-2 selective inhibitor has a selectivity ratio of COX-1 IC₅₀ to COX-2 IC₅₀ not less than about 100.

4. The method of claim 1 wherein the cyclooxygenase-2 selective inhibitor is selected from the group consisting of celecoxib, deracoxib, valdecoxib, rofecoxib, lumiracoxib, etoricoxib, meloxicam, parecoxib, 4-(4-cyclohexyl-2-methyloxazol-5-yl)-2-fluorobenzenesulfonamide, 2-(3,5-difluorophenyl)-3-(4-(methylsulfonyl)phenyl)-2-cyclopenten-1-one, N-[2-(cyclohexyloxy)-4-nitrophenyl]methanesulfonamide, 2-(3,4-difluorophenyl)-4-(3-hydroxy-3-methylbutoxy)-5-[4-(methylsulfonyl)phenyl]-3(2H )-pyridazinone, 2-[(2,4-dichloro-6-methylphenyl)amino]-5-ethyl-benzeneacetic acid, (3Z)-3-[(4-chlorophenyl)[4-(methylsulfonyl)phenyl]methylene]dihydro-2(3H)-furanone, and (S)-6,8-dichloro-2-(trifluoromethyl )-2H-1-benzopyran-3-carboxylic acid.

5. The method of claim 1 wherein the calcium modulating agent is selected from the group consisting of nimodipine, nicardipine, nifedipine, amolodipine, isradipine, diltiazem, verapamil, bepridil, gallopamil, flunarizine, and pimozide.

6. The method of claim 4 wherein the calcium modulating agent is selected from the group consisting of nimodipine, nicardipine, nifedipine, amolodipine, isradipine, diltiazem, verapamil, bepridil, gallopamil, flunarizine, and pimozide.

7. A method of treating pain, inflammation or an inflammation mediated disorder, the method comprising:
(a) diagnosing a subject in need of treatment for pain, inflammation or an inflammation mediated disorder; and
(b) administering to the subject a calcium modulating agent or an isomer, a pharmaceutically acceptable salt, ester, or prodrug thereof and a cyclooxygenase-2 selective inhibitor or an isomer, a pharmaceutically acceptable salt, ester, or prodrug thereof, wherein the cyclooxygenase-2 selective inhibitor is a chromene compound, the chromene compound comprising a benzothiopyran, a dihydroquinoline or a dihydronaphthalene.

8. The method of claim 7 wherein the cyclooxgenase-2 selective inhibitor has a selectivity ratio of COX-1 IC₅₀ to COX-2 IC₅₀ not less than about 50.

9. The method of claim 7 wherein the cyclooxgenase-2 selective inhibitor has a selectivity ratio of COX-1 IC₅₀ to COX-2 IC₅₀ not less than about 100.

10. The method of claim 7 wherein the cyclooxygenase-2 selective inhibitor is a compound having the formula 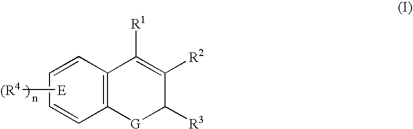
wherein:
n is an integer which is 0, 1, 2, 3 or 4;
G is O, S or NR^{a};
R^{a} is alkyl;
R¹ is selected from the group consisting of H and aryl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from alkylthio, nitro and alkylsulfonyl; and
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or R⁴ together with the carbon atoms to which it is attached and the remainder of ring E forms a naphthyl radical.

11. The method of claim 7 wherein the cyclooxgyenase-2 selective inhibitor is (S)-6,8-dichloro-2-(trifluoromethyl)-2H-1-benzopyran-3-carboxylic acid.

12. The method of claim 7 wherein the calcium modulating agent is selected from the group consisting of nimodipine, nicardipine, nifedipine, amolodipine, isradipine, diltiazem, verapamil, beprildil, gallopamil, flunarizine, and pimozide.

13. A method of treating pain, inflammation or an inflammation mediated disorder, the method comprising:
(a) diagnosing a subject in need of treatment for pain, inflammation or an inflammation mediated disorder; and
(b) administering to the subject a calcium modulating agent or an isomer, a pharmaceutically acceptable salt, ester, or prodrug thereof and a cyclooxygenase-2 selective inhibitor or an isomer, a pharmaceutically acceptable salt, ester, or prodrug thereof, wherein the cyclooxygenase-2 selective inhibitor is a tricyclic compound, the tricyclic compound comprising a benzenesulfonamide or methylsulfonylbenzene.

14. The method of claim 13 wherein the cyclooxgenase-2 selective inhibitor has a selectivity ratio of COX-1 IC₅₀ to COX-2 IC₅₀ not less than about 50.

15. The method of claim 13 wherein the cyclooxgenase-2 selective inhibitor has a selectivity ratio of COX-1 IC₅₀ to COX-2 IC₅₀ not less than about 100.

16. The method of claim 13 wherein the cyclooxygenase-2 selective inhibitor is a compound of the formula: 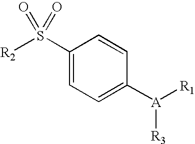
wherein:
A is selected from the group consisting of partially unsaturated or unsaturated heterocyclyl and partially unsaturated or unsaturated carbocyclic rings;
R₁ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R₂ is selected from the group consisting of methyl and amino; and
R₃ is selected from the group consisting of H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, and N-alkyl-N-arylaminosulfonyl.

17. The method of claim 13 wherein the cyclooxygenase-2 selective inhibitor is selected from the group consisting of celecoxib, valdecoxib, parecoxib, deracoxib, rofecoxib, etoricoxib, and 2-(3,4-difluorophenyl)-4-(3-hydroxy-3-methylbutoxy)-5-[4-(methylsulfonyl)phenyl]-3(2H)-pyridazinone.

18. The method of claim 13 wherein the calcium modulating agent is selected from the group consisting of nimodipine, nicardipine, nifedipine, amolodipine, isradipine, diltiazem, verapamil, beprildil, gallopamil, flunarizine, and pimozide.

19. A method of treating pain, inflammation or an inflammation mediated disorder, the method comprising:
(a) diagnosing a subject in need of treatment for pain, inflammation or an inflammation mediated disorder; and
(b) administering to the subject a calcium modulating agent or an isomer, a pharmaceutically acceptable salt, ester, or prodrug thereof and a cyclooxygenase-2 selective inhibitor or an isomer, a pharmaceutically acceptable salt, ester, or prodrug thereof, wherein the cyclooxygenase-2 selective inhibitor is a phenyl acetic acid compound.

20. The method of claim 19 wherein the cyclooxgenase-2 selective inhibitor has a selectivity ratio of COX-1 IC₅₀ to COX-2 IC₅₀ not less than about 50.

21. The method of claim 19 wherein the cyclooxgenase-2 selective inhibitor has a selectivity ratio of COX-1 IC₅₀ to COX-2 IC₅₀ not less than about 100.

22. The method of claim 19 wherein the cyclooxygenase-2 selective inhibitor is a compound having the formula: 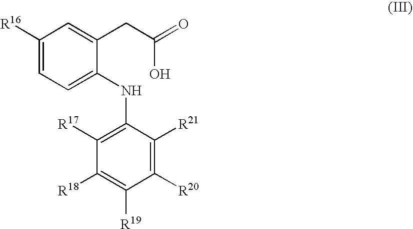
wherein:
R₁₆ is methyl or ethyl;
R₁₇ is chloro or fluoro;
R₁₈ is hydrogen or fluoro;
R₁₉ is hydrogen, fluoro, chloro, methyl, ethyl, methoxy, ethoxy or hydroxy;
R₂₀ is hydrogen or fluoro; and
R₂₁ is chloro, fluoro, trifluoromethyl or methyl; and
provided that each of R₁₇, R₁₈, R₁₉ and R₂₀ is not fluoro when R₁₆ is ethyl and R₁₉ is H.

23. The method of claim 22 wherein:
R₁₆ is ethyl;
R₁₇ and R₁₉ are chloro;
R₁₈ and R₂₀ are hydrogen; and
R₂₁ is methyl.

24. The method of claim 19 wherein the calcium modulating agent is selected from the group consisting of nimodipine, nicardipine, nifedipine, amolodipine, isradipine, diltiazem, verapamil, beprildil, gallopamil, flunarizine, and pimozide.

25. A method of treating pain, inflammation or an inflammation mediated disorder, the method comprising:
(a) diagnosing a subject in need of treatment for pain, inflammation or an inflammation mediated disorder; and
(b) administering to the subject a cyclooxygenase-2 selective inhibitor selected from the group consisting of celecoxib, deracoxib, valdecoxib, rofecoxib, lumiracoxib, etoricoxib, parecoxib, 2-(3,4-difluorophenyl)-4-(3-hydroxy-3-methylbutoxy)-5-[4-(methylsulfonyl)phenyl]-3(2H)-pyridazinone, and (S)-6,8-dichloro-2-(trifluoromethyl)-2H-1-benzopyran-3-carboxylic acid; and
a calcium modulating agent selected from the group consisting of nimodipine, nicardipine, nifedipine, amolodipine, isradipine, diltiazem, verapamil, beprildil, gallopamil, flunarizine, and pimozide.

26. The method of claim 25 wherein the cyclooxygenase-2 selective inhibitor is celecoxib.

27. The method of claim 25 wherein the cyclooxygenase-2 selective inhibitor is deracoxib.

28. The method of claim 25 wherein the cyclooxygenase-2 selective inhibitor is valdecoxib.

29. The method of claim 25 wherein the cyclooxygenase-2 selective inhibitor is rofecoxib.

30. The method of claim 25 wherein the cyclooxygenase-2 selective inhibitor is etoricoxib.

31. The method of claim 25 wherein the cyclooxygenase-2 selective inhibitor is parecoxib.

32. The method of claim 25 wherein the cyclooxygenase-2 selective inhibitor is 2-(3,4-difluorophenyl)-4-(3-hydroxy-3-methylbutoxy)-5-[4-(methylsulfonyl)phenyl]-3(2H)-pyridazinone.

33. The method of claim 25 wherein the cyclooxygenase-2 selective inhibitor is (S)-6,8-dichloro-2-(trifluoromethyl)-2H-1-benzopyran-3-carboxylic acid.

34. The method of claim 25 wherein the cyclooxygenase-2 selective inhibitor is lumiracoxib.

35. The method of claim 1 wherein the inflammation mediated disorder is arthritis.

36. The method of claim 1 wherein the inflammation mediated disorder is a gastrointestinal disorder.

37. The method of claim 36 wherein the gastrointestinal disorder is selected from the group consisting of inflammatory bowel disease, Crohn's disease, gastritis, irritable bowel syndrome and ulcerative colitis.


SEQUENCE LISTING (26 sequences)

SEQ ID NO: 1 - protein, 1873 aa, Homo sapiens
MEPSSPQDEGLRKKQPKKPVPEILPSPPRALFCLTLENPLRKACISIVEWKPFETIILLTIFANCVALAVYLPMPEDDNNSLNLGLEKLEYFFLIVFSIEAAMKIIAYGFLFHQDAYLRSGWNVLDFTIVFLGVFTVILEQVNVIQSHTAPMSSKGAGLDVKALRAFRVLRPLRLVSGVPSLQVVLNSIFKAMLPLFHIALLVLFMVIIYAIIGLELFKGKMHKTCYFIGTDIVATVENEEPSPCARTGSGRRCTINGSECRGGWPGPNHGITHFDNFGFSMLTVYQCITMEGWTDVLYWVNDAIGNEWPWIYFVTLILLGSFFILNLVLGVLSGEFTKEREKAKSRGTFQKLREKQQLDEDLRGYMSWITQGEVMDVEDFREGKLSLDEGGSDTESLYEIAGLNKIIQFIRHWRQWNRIFRWKCHDIVKSKVFYWLVILIVALNTLSIASEHHNQPLWLTRLQDIANRVLLSLFTTEMLMKMYGLGLRQYFMSIFNRFDCFVVCSGILEILLVESGAMTPLGISVLRCIRLLRIFKITKYWTSLSNLVASLLNSIRSIASLLLLLFLFIVIFALLGMQLFGGRYDFEDTEVRRSNFDNFPQALISVFQVLTGEDWTSMMYNGIMAYGGPSYPGMLVCIYFIILFVCGNYILLNVFLAIAVDNLAEAESLTSAQKAKAEEKKRRKMSKGLPDKSEEEKSTMAKKLEQKPKGEGIPTTAKLKIDEFESNVNEVKDPYPSADFPGDDEEDEPEIPLSPRPRPLAELQLKEKAVPIPEASSFFIFSPTNKIRVLCHRIVNATWFTNFILLFILLSSAALAAEDPIRADSMRNQILKHFDIGFTSVFTVEIVLKMTTYGAFLHKGSFCRNYFNMLDLLVVAVSLISMGLESSAISVVKILRVLRVLRPLRAINRAKGLKHVVQCMFVAISTIGNIVLVTTLLQFMFACIGVQLFKGKFFRCTDLSKMTEEECRGYYYVYKDGDPMQIELRHREWVHSDFHFDNVLSAMMSLFTVSTFEGWPQLLYKAIDSNAEDVGPIYNNRVEMAIFFIIYIILIAFFMMNIFVGFVIVTFQEQGETEYKNCELDKNQRQCVQYALKARPLRCYIPKNPYQYQVWYIVTSSYFEYLMFALIMLNTICLGMQHYNQSEQMNHISDILNVAFTIIFTLEMILKLMAFKARGYFGDPWNVFDFLIVIGSIIDVILSEIDTFLASSGGLYCLGGGCGNVDPDESARISSAFFRLFRVMRLIKLLSRAEGVRTLLWTFIKSFQALPYVALLIVMLFFIYAVIGMQMFGKIALVDGTQINRNNNFQTFPQAVLLLFRCATGEAWQEILLACSYGKLCDPESDYAPGEEYTCGTNFAYYYFISFYMLCAFLVINLFVAVIMDNFDYLTRDWSILGPHHLDEFKAIWAEYDPEAKGRIKHLDVVTLLRRIQPPLGFGKFCPHRVACKRLVGMNMPLNSDGTVTFNATLFALVGTALKIKTEGNFEQANEELRAIIKKIWKRTSMKLLDQVMPPIGDDEVTVGKFYATFLIQEDFRKFMKRQEEYYGYRPKKDIVQIQAGLRTIEEEAAPEICRTVSGDLAAEEELERAMVEAAMEEGIFRRTGGLFGQVDNFLERTNSLPPVMANQRPLQFAEIEMEEMESPVFLEDFPQDPRTNPLARANTNNANANVAYGNSNHSNSHVFSSVHYEREFPEETETPATRGRALGQPCRVLGPHSKPCVEMLKGLLTQRAMPRGQAPPAPCQCPRVESSMPEDRKSSTPGSLHEETPHSRSTRENTSRCSAPATALLIQKALVRGGLGTLAADANFIMATGQALGDACQMEPEEVEIMATELLKGREAPEGMASSLGCLNLGSSLGSLDQHQGSQETLIPPRL

SEQ ID NO: 2 - protein, 1873 aa, Homo sapiens
MEPSSPQDEGLRKKQPKKPVPEILPRPPRALFCLTLENPLRKACISIVEWKPFETIILLTIFANCVALAVYLPMPEDDNNSLNLGLEKLEYFFLIVFSIEAAMKIIAYGFLFHQDAYLRSGWNVLDFTIVFLGVFTVILEQVNVIQSHTAPMSSKGAGLDVKALRAFRVLRPLRLVSGVPSLQVVLNSIFKAMLPLFHIALLVLFMVIIYAIIGLELFKGKMHKTCYFIGTDIVATVENEEPSPCARTGSGRRCTINGSECRGGCPGPNHGITHFDNFGFSMLTVYQCITMEGWTDVLYWVNDAIGNEWPWIYFVTLILLGSFFILNLVLGVLSGEFTKEREKAKSRGTFQKLREKQQLDEDLRGYMSWITQGEVMDVEDFREGKLSLDEGGSDTESLYEIAGLNKIIQFIRHWRQWNRIFRWKCHDIVKSKVFYWLVILIVALNTLSIASEHHNQPHWLTRLQDIANRVLLSLFTTEMLMKMYGLGLRQYFMSIFNRFDCFVVCSGILEILLVESGAMTPLGISVLRCIRLLRIFKITKYWTSLSNLVASLLNSIRSIASLLLLLFLFIVIFRLLGMQLFGGRYDFEDTEVRRSNFDNFPQALISVFQVLTGEDWTSMMYNGIMASSGPSYPGMLVCIYFIILFVCGNYILLNVFLAIAVDNLAEAESLTSAQKAKAEEKKRRKMSKGLPDKSEEEKSTMAKKLEQKPKGEGIPTTAKLKIDEFESNVNEVKDPYPSADFPGDDEEDEPEIPLSPRPRPLAELQLKEKAVPIPEASSFFIFSPTNKIRVLCHRIVNATWFTNFILLFILLSSAALAAEDPIRADSMRNQILKHFDIGFTSVFTVEIVLKMTTYGAFLHKGSFCRNYFNMLDLLVVAVSLISMGLESSAISVVKILRVLRVLRPLRAINRAKGLKHVARCMFVAISTIGNIVLVTTLLQFMFACIGVQLFKGKFFRCTDLSKMTEEECRGYYYVYKDGDPMQIELRHREWVHSDFHFDNVLSAMMSLFTVSTFEGWPQLLYKAIDSNAEDVGPIYNNRVEMAIFFIIYIILIAFFMMNIFVGFVIVTFQEQGETEYKNCELDKNQRQCVQYALKARPLRCYIPKNPYQYQVWYIVTSSYFEYLMFALIMLNTICLGMQHYNQSEQMNHISDILNVAFTIIFTLEMILKLMAFKARGYFGNPWNVFDFLIVIGSIIDVILSEIDTFLASSGGLYCLGGGCGNVDPDESARISSAFFRLFRVMRLIKLLSRAEGVRTLLWTFIKSFQALPYVALLIVMLFFIYAVIGMQMFGKIALVDGTQINRNNNFQTFPQAVLLLFRCATGEAWQEILLACSYGKLCDPESDYAPGEEYTCGTNFAYYYFISFYMLCAFLVINLFVAVIMDNFDYLTRDWSILGPHHLDEFKAIWAEYDPEAKGRIKHLDVVTLLRRIQPPLGFGKFCPHRVACKRLVGMNMPLNSDGTVTFNATLFALVRTALKIKTEGNFEQANEELRAIIKKIWKRTSMKLLDQVIPPIGDDEVTVGKFYATFLIQEHFRKFMKRQEEYYGYRPKKDIVQIQAGLRTIEEEAAPEICRTVSGDLAAEEELERAMVEAAMEEGIFRRTGGLFGQVDNFLERTNSLPPVMANQRPLQFAEIEMEEMESPVFLEDFPQDPRTNPLARANTNNANANVAYANSNHSNSHVFSSVHYEREFPEETETPATRGRALGQPCRSLGPHSKPCVEMLKGLLTQRAMPRGQAPPAPCQCPRVESSMPEDRKSSTPGSLHEETPHSRSTRENTSRCSAPATALLIQKALVRGGLGTLAADANFIMATGQALGDACQMEPEEVEIMATELLKGREAPDGMASSLGCLNLGSSLGSLDQHQGSQETLIPPRL

SEQ ID NO: 3 - protein, 2221 aa, Homo sapiens
MVNENTRMYIPEENHQGSNYGSPRPAHANMNANAAAGLAPEHIPTPGAALSWQAAIDAARQAKLMGSAGNATISTVSSTQRKRQQYGKPKKQGSTTATRPPRALLCLTLKNPIRRACISIVEWKPFEIIILLTIFANCVALAIYIPFPEDDSNATNSNLERVEYLFLIIFTVEAFLKVIAYGLLFHPNAYLRNGWNLLDFIIVVVGLFSAILEQATKADGANALGGKGAGFDVKALRAFRVLRPLRLVSGVPSLQVVLNSIIKAMVPLLHIALLVLFVIIIYAIIGLELFMGKMHKTCYNQEGIADVPAEDDPSPCALETGHGRQCQNGTVCKPGWDGPKHGITNFDNFAFAMLTVFQCITMEGWTDVLYWVNDAVGRDWPWIYFVTLIIIGSFFVLNLVLGVLSGEFSKEREKAKARGDFQKLREKQQLEEDLKGYLDWITQAEDIDPENEDEGMDEEKPRNMSMPTSETESVNTENVAGGDIEGENCGARLAHRISKSKFSRYWRRWNRFCRRKCRAAVKSNVFYWLVIFLVFLNTLTIASEHYNQPNWLTEVQDTANKALLALFTAEMLLKMYSLGLQAYFVSLFNRFDCFVVCGGILETILVETKIMSPLGISVLRCVRLLRIFKITRYWNSLSNLVASLLNSVRSIASLLLLLFLFIIIFSLLGMQLFGGKFNFDEMQTRRSTFDNFPQSLLTVFQILTGEDWNSVMYDGIMAYGGPSFPGMLVCIYFIILFICGNYILLNVFLAIAVDNLADAESLTSAQKEEEEEKERKKLARTASPEKKQELVEKPAVGESKEEKIELKSITADGESPPATKINMDDLQPNENEDKSPYPNPETTGEEDEEEPEMPVGPRPRPLSELHLKEKAVPMPEASAFFIFSSNNRFRLQCHRIVNDTIFTNLILFFILLSSISLAAEDPVQHTSFRNHILFYFDIVFTTIFTIEIALKILGNADYVFTSIFTLEIILKMTAYGAFLHKGSFCRNYFNILDLLVVSVSLISFGIQSSAINVVKILRVLRVLRPLRAINRAKGLKHVVQCVFVAIRTIGNIVIVTTLLQFMFACIGVQLFKGKLYTCSDSSKQTEAECKGNYITYKDGEVDHPIIQPRSWENSKFDFDNVLAAMMALFTVSTFEGWPELLYRSIDSHTEDKGPIYNYRVEISIFFIIYIIIIAFFMMNIFVGFVIVTFQEQGEQEYKNCELDKNQRQCVEYALKARPLRRYIPKNQHQYKVWYVVNSTYFEYLMFVLILLNTICLAMQHYGQSCLFKIAMNILNMLFTGLFTVEMILKLIAFKPKGYFSDPWNVFDFLIVIGSIIDVILSETNHYFCDAWNTFDALIVVGSIVDIAITEVNPAEHTQCSPSMNAEENSRISITFFRLFRVMRLVKLLSRGEGIRTLLWTFIKSFQALPYVALLIVMLFFIYAVIGMQVFGKIALNDTTEINRNNNFQTFPQAVLLLFRCATGEAWQDIMLACMPGKKCAPESEPSNSTEGETPCGSSFAVFYFISFYMLCAFLIINLFVAVIMDNFDYLTRDWSILGPHHLDEFKRIWAEYDPEAKGRIKHLDVVTLLRRIQPPLGFGKLCPHRVACKRLVSMNMPLNSDGTVMFNATLFALVRTALRIKTEGNLEQANEELRAIIKKIWKRTSMKLLDQVVPPAGDDEVTVGKFYATFLIQEYFRKFKKRKEQGLVGKPSQRNALSLQAGLRTLHDIGPEIRRAISGDLTAEEELDKAMKEAVSAASEDDIFRRAGGLFGNHVSYYQSDGRSAFPQTFTTQRPLHINKAGSSQGDTESPSHEKLVDSTFTPSSYSSTGSNANINNANNTALGRLPRPAGYPSTVSTVEGHGPPLSPAIRVQEVAWKLSSNRERHVPVCEDLELRRDSGSAGTQAHCLLLRRANPSRCHSRESQAAMAGQEETSQDETYEVKMNHDTEACSEPSLLSTEMLSYQDDENRQLTLPEEDKRDIRQSPKRGFLRSASLGRRASFHLECLKRQKDRGGDISQKTVLPLHLVHHQALAVAGLSPLLQRSHSPASFPRPFATPPATPGSRGWPPQPVPTLRLEGVESSEKLNSSFPSIHCGSWAETTPGGGGSSAARRVRPVSLMVPSQAGAPGRQFHGSASSLVEAVLISEGLGQFAQDPKFIEVTTQELADACDMTIEEMESAADNILSGGAPQSPNGALLPFVNCRDAGQDRAGGEEDAGCVRARGAPSEEELQDSRVYVSSL

SEQ ID NO: 4 - protein, 2161 aa, Homo sapiens [sequence omitted]

SEQ ID NO: 5 - protein, 1966 aa, Homo sapiens
MSESEGGKDTTPEPSPANGAGPGPEWGLCPGPPAVEGESSGASGLGTPKRRNQHSKHKTVAVASAQRSPRALFCLTLANPLRRSCISIVEWKPFDILILLTIFANCVALGVYIPFPEDDSNTANHNLEQVEYVFLVIFTVETVLKIVAYGLVLHPSAYIRNGWNLLDFIIVVVGLFSVLLEQGPGRPGDAPHTGGKPGGFDVKALRAFRVLRPLRLVSGVPSLHIVLNSIMKALVPLLHIALLVLFVIIIYAIIGLELFLGRMHKTCYFLGSDMEAEEDPSPCASSGSGRACTLNQTECRGRWPGPNGGITNFDNFFFAMLTVFQCVTMEGWTDVLYWMQDAMGYELPWVYFVSLVIFGSFFVLNLVLGVLSGEFSKEREKAKARGDFQKQREKQQMEEDLRGYLDWITQAEELDMEDPSADDNLGPQLAELTNRRRGRLRWFSHSTRSTHSTSSHASLPASDTGSMTETQGDEDEEEGALASCTRCLNKIMKTRVCRRLRRANRVLRARCRRAVKSNACYWAVLLLVFLNTLTIASEHHGQPVWLTQIQEYANKVLLCLFTVEMLLKLYGLGPSAYVSSFFNRFDCFVVCGGILETTLVEVGAMQPLGISVLRCVRLLRIFKVTRHWASLSNLVASLLNSMKSIASLLLLLFLFIIIFSLLGMQLFGGKFNFDQTHTKRSTFDTFPQALLTVFQILTGEDWNVVMYDGIMAYGGPFFPGMLVCIYFIILFICGNYILLNVFLAIAVDNLASGDAGTAKDKGGEKSNEKDLPQENEGLVPGVEKEEEEGARREGADMEEEEEEEEEEEEEEEEEGAGGVELLQEVVPKEKVVPIPEGSAFFCLSQTNPLRKGCHTLIHHHVFTNLILVFIILSSVSLAAEDPIRAHSFRNHILGYFDYAFTSIFTVEILLKMTVFGAFLHRGSFCRSWFNMLDLLVVSVSLISFGIHSSAISVVKILRVLRVLRPLRAINRAKGLKHVVQCVFVAIRTIGNIMIVTTLLQFMFACIGVQLFKGKFYTCTDEAKHTPQECKGSFLVYPDGDVSRPLVRERLWVNSDFNFDNVLSAMMALFTVSTFEGWPALLYKAIDAYAEDHGPIYNYRVEISVFFIVYIIIIAFFMMNIFVGFVIITFRAQGEQEYQNCELDKNQRQCVEYALKAQPLRRYIPKNPHQYRVWATVNSAAFEYLMFLLILLNTVALAMQHYEQTAPFNYAMDILNMVFTGLFTIEMVLKIIAFKPKHYFTDAWNTFDALIVVGSIVDIAVTEVNNGGHLGESSEDSSRISITFFRLFRVMRLVKLLSKGEGIRTLLWTFIKSFQALPYVALLIAMIFFIYAVIGMQMFGKVALQDGTQINRNNNFQTFPQAVLLLFRCATGEAWQEIMLASLPGNRCDPESDFGPGEEFTCGSNFAIAYFISFFMLCAFLIINLFVAVIMDNFDYLTRDWSILGPHHLDEFKRIWSEYDPGAKGRIKHLDVVALLRRIQPPLGFGKLCPHRVACKRLVAMNMPLNSDGTVTFNATLFALVRTSLKIKTEGNLEQANQELRIVIKKIWKRMKQKLLDEVIPPPDEEEVTVGKFYATFLIQDYFRKFRRRKEKGLLGNDAAPSTSSALQAGLRSLQDLGPEMRQALTCDTEEEEEEGQEGVEEEDEKDLETNKATMVSQPSARRGSGISVSLPVGDRLPDSLSFGPSDDDRGTPTSSQPSVPQAGSNTHRRGSGALIFTIPEEGNSQPKGTKGQNKQDEDEEVPDRLSYLDEQAGTPPCSVLLPPHRAQRYMDGHLVPRRRLLPPTPAGRKPSFTIQCLQRQGSCEDLPIPGTYHRGRNSGPNRAQGSWATPPQRGRLLYAPLLLVEEGAAGEGYLGRSSGPLRTFTCLHVPGTHSDPSHGKRGSADSLVEAVLISEGLGLFARDPRFVALAKQEIADACRLTLDEMDNAASDLLAQGTSSLYSDEESILSRFDEEDLGDEMACVHAL

SEQ ID NO: 6 - protein, 1966 aa, Homo sapiens; identical to SEQ ID NO: 5

SEQ ID NO: 7 - protein, 1977 aa, Homo sapiens
MSESEGGKDTTPEPSPANGAGPGPEWGLCPGPPAVEGESSGASGLGTPKRRNQHSKHKTVAVASAQRSPRALFCLTLANPLRRSCISIVEWKPFDILILLTIFANCVALGVYIPFPEDDSNTANHNLEQVEYVFLVIFTVETVLKIVAYGLVLHPSAYIRNGWNLLDFIIVVVGLFSVLLEQGPGRPGDAPHTGGKPGGFDVKALRAFRVLRPLRLVSGVPSLHIVLNSIMKALVPLLHIALLVLFVIIIYAIIGLELFLGRMHKTCYFLGSDMEAEEDPSPCASSGSGRACTLNQTECRGRWPGPNGGITNFDNFFFAMLTVFQCVTMEGWTDVLYWMQDAMGYELPWVYFVSLVIFGSFFVLNLVLGVLSGEFSKEREKAKARGDFQKQREKQQMEEDLRGYLDWITQAEELDMEDPSADDNLGSMAEEGRAGHRPQLAELTNRRRGRLRWFSHSTRSTHSTSSHASLPASDTGSMTETQGDEDEEEGALASCTRCLNKIMKTRVCRRLRRANRVLRARCRRAVKSNACYWAVLLLVFLNTLTIASEHHGQPVWLTQIQEYANKVLLCLFTVEMLLKLYGLGPSAYVSSFFNRFDCFVVCGGILETTLVEVGAMQPLGISVLRCVRLLRIFKVTRHWASLSNLVASLLNSMKSIASLLLLLFLFIIIFSLLGMQLFGGKFNFDQTHTKRSTFDTFPQALLTVFQILTGEDWNVVMYDGIMAYGGPFFPGMLVCIYFIILFICGNYILLNVFLAIAVDNLASGDAGTAKDKGGEKSNEKDLPQENEGLVPGVEKEEEEGARREGADMEEEEEEEEEEEEEEEEEGAGGVELLQEVVPKEKVVPIPEGSAFFCLSQTNPLRKGCHTLIHHHVFTNLILVFIILSSVSLAAEDPIRAHSFRNHILGYFDYAFTSIFTVEILLKMTVFGAFLHRGSFCRSWFNMLDLLVVSVSLISFGIHSSAISVVKILRVLRVLRPLRAINRAKGLKHVVQCVFVAIRTIGNIMIVTTLLQFMFACIGVQLFKGKFYTCTDEAKHTPQECKGSFLVYPDGDVSRPLVRERLWVNSDFNFDNVLSAMMALFTVSTFEGWPALLYKAIDAYAEDHGPIYNYRVEISVFFIVYIIIIAFFMMNIFVGFVIITFRAQGEQEYQNCELDKNQRQCVEYALKAQPLRRYIPKNPHQYRVWATVNSAAFEYLMFLLILLNTVALAMQHYEQTAPFNYAMDILNMVFTGLFTIEMVLKIIAFKPKHYFTDAWNTFDALIVVGSIVDIAVTEVNNGGHLGESSEDSSRISITFFRLFRVMRLVKLLSKGEGIRTLLWTFIKSFQALPYVALLIAMIFFIYAVIGMQMFGKVALQDGTQINRNNNFQTFPQAVLLLFRCATGEAWQEIMLASLPGNRCDPESDFGPGEEFTCGSNFAIAYFISFFMLCAFLIINLFVAVIMDNFDYLTRDWSILGPHHLDEFKRIWSEYDPGAKGRIKHLDVVALLRRIQPPLGFGKLCPHRVACKRLVAMNMPLNSDGTVTFNATLFALVRTSLKIKTEGNLEQANQELRIVIKKIWKRMKQKLLDEVIPPPDEEEVTVGKFYATFLIQDYFRKFRRRKEKGLLGNDAAPSTSSALQAGLRSLQDLGPEMRQALTCDTEEEEEEGQEGVEEEDEKDLETNKATMVSQPSARRGSGISVSLPVGDRLPDSLSFGPSDDDRGTPTSSQPSVPQAGSNTHRRGSGALIFTIPEEGNSQPKGTKGQNKQDEDEEVPDRLSYLDEQAGTPPCSVLLPPHRAQRYMDGHLVPRRRLLPPTPAGRKPSFTIQCLQRQGSCEDLPIPGTYHRGRNSGPNRAQGSWATPPQRGRLLYAPLLLVEEGAAGEGYLGRSSGPLRTFTCLHVPGTHSDPSHGKRGSADSLVEAVLISEGLGLFARDPRFVALAKQEIADACRLTLDEMDNAASDLLAQGTSSLYSDEESILSRFDEEDLGDEMACVHAL

SEQ ID NO: 8 - protein, 2339 aa, Homo sapiens [sequence omitted]

SEQ ID NO: 9 - protein, 2505 aa, Homo sapiens
MARFGDEMPARYGGGGSGAAAGVVVGSGGGRGAGGSRQGGQPGAQRMYKQSMAQRARTMALYNPIPVRQNCLTVNRSLFLFSEDNVVRKYAKKITEWPPFEYMILATIIANCIVLALEQHLPDDDKTPMSERLDDTEPYFIGIFCFEAGIKIIALGFAFHKGSYLRNGWNVMDFVVVLTGILATVGTEFDLRTLRAVRVLRPLKLVSGIPSLQVVLKSIMKAMIPLLQIGLLLFFAILIFAIIGLEFYMGKFHTTCFEEGTDDIQGESPAPCGTEEPARTCPNGTKCQPYWEGPNNGITQFDNILFAVLTVFQCITMEGWTDLLYNSNDASGNTWNWLYFIPLIIIGSFFMLNLVLGVLSGEFAKERERVENRRAFLKLRRQQQIERELNGYMEWISKAEEVILAEDETDGEQRHPFDGALRRTTIKKSKTDLLNPEEAEDQLADIASVGSPFARASIKSAKLENSTFFHKKERRMRFYIRRMVKTQAFYWTVLSLVALNTLCVAIVHYNQPEWLSDFLYYAEFIFLGLFMSEMFIKMYGLGTRPYFHSSFNCFDCGVIIGSIFEVIWAVIKPGTSFGISVLRALRLLRIFKVTKYWASLRNLVVSLLNSMKSIISLLFLLFLFIVVFALLGMQLFGGQFNFDEGTPPTNFDTFPAAIMTVFQILTGEDWNEVMYDGIKSQGGVQGGMVFSIYFIVLTLFGNYTLLNVFLAIAVDNLANAQELTKDEQEEEEAANQKLALQKAKEVAEVSPLSAANMSIAVKEQQKNQKPAKSVWEQRTSEMRKQNLLASREALYNEMDPDERWKAAYTRHLRPDMKTHLDRPLVVDPQENRNNNTNKSRAAEPTVDQRLGQQRAEDFLRKQARYHDRARDPSGSAGLDARRPWAGSQEAELSREGPYGRESDHHAREGSLEQPGFWEGEAERGKAGDPHRRHVHRQGGSRESRSGSPRTGADGEHRRHRAHRRPGEEGPEDKAERRARHREGSRPARGGEGEGEGPDGGERRRRHRHGAPATYEGDARREDKERRHRRRKENQGSGVPVSGPNLSTTRPIQQDLGRQDPPLAEDIDNMKNNKLATAESAAPHGSLGHAGLPQSPAKMGNSTDPGPMLAIPAMATNPQNAASRRTPNNPGNPSNPGPPKTPENSLIVTNPSGTQTNSAKTARKPDHTTVDIPPACPPPLNHTVVQVNKNANPDPLPKKEEEKKEEEEDDRGEDGPKPMPPYSSMFILSTTNPLRRLCHYILNLRYFEMCILMVIAMSSIALAAEDPVQPNAPRNNVLRYFDYVFTGVFTFEMVIKMIDLGLVLHQGAYFRDLWNILDFIVVSGALVAFAFTGNSKGKDINTIKSLRVLRVLRPLKTIKRLPKLKAVFDCVVNSLKNVFNILIVYMLFMFIFAVVAVQLFKGKFFHCTDESKEFEKDCRGKYLLYEKNEVKARDREWKKYEFHYDNVLWALLTLFTVSTGEGWPQVLKHSVDATFENQGPSPGYRMEMSIFYVVYFVVFPFFFVNIFVALIIITFQEQGDKMMEEYSLEKNERACIDFAISAKPLTRHMPQNKQSFQYRMWQFVVSPPFEYTIMAMIALNTIVLMMKFYGASVAYENALRVFNIVFTSLFSLECVLKVMAFGILNYFRDAWNIFDFVTVLGSITDILVTEFGNNFINLSFLRLFRAARLIKLLRQGYTIRILLWTFVQSFKALPYVCLLIAMLFFIYAIIGMQVFGNIGIDVEDEDSDEDEFQITEHNNFRTFFQALMLLFRSATGEAWHNIMLSCLSGKPCDKNSGILTRECGNEFAYFYFVSFIFLCSFLMLNLFVAVIMDNFEYLTRDSSILGPHHLDEYVRVWAEYDPAAWGRMPYLDMYQMLRHMSPPLGLGKKCPARVAYKRLLRMDLPVADDNTVHFNSTLMALIRTALDIKIAKGGADKQQMDAELRKEMMAIWPNLSQKTLDLLVTPHKSTDLTVGKIYAAMMIMEYYRQSKAKKLQAMREEQDRTPLMFQRMEPPSPTQEGGPGQNALPSTQLDPGGALMAHESGLKESPSWVTQRAQEMFQKTGTWSPEQGPPTDMPNSQPNSQSVEMREMGRDGYSDSEHYLPMEGQGRAASMPRLPAENQRRRGRPRGNNLSTISDTSPMKRSASVLGPKARRLDDYSLERVPPEENQRHHQRRRDRSHRASERSLGRYTDVDTGLGTDLSMTTQSGDLPSKERDQERGRPKDRKHRQHHHHHHHHHHPPPPDKDRYAQERPDHGRARARDQRWSRSPSEGREHMAHRQGSSSVSGSPAPSTSGTSTPRRGRRQLPQTPSTPRPHVSYSPVIRKAGGSGPPQQQQQQQQQQQAVARPGRAATSGPRRYPGPTAEPLAGDRPPTGGHSSGRSPRMERRVPGPARSESPRACRHGGARWPASGPHVSEGPPGPRHHGYYRGSDYDEADGPGSGGGEEAMAGAYDAPPPVRHASSGATGRSPRTPRASGPACASPSRHGRRLPNGYYPAHGLARPRGPGSRKGLHEPYSESDDDWC

SEQ ID NO: 10 - protein, 2261 aa, Homo sapiens
MARFGDEMPARYGGGGSGAAAGVVVGSGGGRGAGGSRQGGQPGAQRMYKQSMAQRARTMALYNPIPVRQNCLTVNRSLFLFSEDNVVRKYAKKITEWPPFEYMILATIIANCIVLALEQHLPDDDKTPMSERLDDTEPYFIGIFCFEAGIKIIALGFAFHKGSYLRNGWNVMDFVVVLTGILATVGTEFDLRTLRAVRVLRPLKLVSGIPSLQVVLKSIMKAMIPLLQIGLLLFFAILIFAIIGLEFYMGKFHTTCFEEGTDDIQGESPAPCGTEEPARTCPNGTKCQPYWEGPNNGITQFDNILFAVLTVFQCITMEGWTDLLYNSNDASGNTWNWLYFIPLIIIGSFFMLNLVLGVLSGEFAKERERVENRRAFLKLRRQQQIERELNGYMEWISKAEEVILAEDETDGEQRHPFDGALRRTTIKKSKTDLLNPEEAEDQLADIASVGSPFARASIKSAKLENSTFFHKKERRMRFYIRRMVKTQAFYWTVLSLVALNTLCVAIVHYNQPEWLSDFLYYAEFIFLGLFMSEMFIKMYGLGTRPYFHSSFNCFDCGVIIGSIFEVIWAVIKPGTSFGISVLRALRLLRIFKVTKYWASLRNLVVSLLNSMKSIISLLFLLFLFIVVFALLGMQLFGGQFNFDEGTPPTNFDTFPAAIMTVFQILTGEDWNEVMYDGIKSQGGVQGGMVFSIYFIVLTLFGNYTLLNVFLAIAVDNLANAQELTKDEQEEEEAANQKLALQKAKEVAEVSPLSAANMSIAVKEQQKNQKPAKSVWEQRTSEMRKQNLLASREALYNEMDPDERWKAAYTRHLRPDMKTHLDRPLVVDPQENRNNNTNKSRAAEPTVDQRLGQQRAEDFLRKQARYHDRARDPSGSAGLDARRPWAGSQEAELSREGPYGRESDHHAREGSLEQPGFWEGEAERGKAGDPHRRHVHRQGGSRESRSGSPRTGADGEHRRHRAHRRPGEEGPEDKAERRARHREGSRPARGGEGEGEGPDGGERRRRHRHGAPATYEGDARREDKERRHRRRKENQGSGVPVSGPNLSTTRPIQQDLGRQDPPLAEDIDNMKNNKLATAESAAPHGSLGHAGLPQSPAKMGNSTDPGPMLAIPAMATNPQNAASRRTPNNPGNPSNPGPPKTPENSLIVTNPSGTQTNSAKTARKPDHTTVDIPPACPPPLNHTVVQVNKNANPDPLPKKEEEKKEEEEDDRGEDGPKPMPPYSSMFILSTTNPLRRLCHYILNLRYFEMCILMVIAMSSIALAAEDPVQPNAPRNNVLRYFDYVFTGVFTFEMVIKMIDLGLVLHQGAYFRDLWNILDFIVVSGALVAFAFTGNSKGKDINTIKSLRVLRVLRPLKTIKRLPKLKAVFDCVVNSLKNVFNILIVYMLFMFIFAVVAVQLFKGKFFHCTDESKEFEKDCRGKYLLYEKNEVKARDREWKKYEFHYDNVLWALLTLFTVSTGEGWPQVLKHSVDATFENQGPSPGYRMEMSIFYVVYFVVFPFFFVNIFVALIIITFQEQGDKMMEEYSLEKNERACIDFAISAKPLTRHMPQNKQSFQYRMWQFVVSPPFEYTIMAMIALNTIVLMMKFYGASVAYENALRVFNIVFTSLFSLECVLKVMAFGILNYFRDAWNIFDFVTVLGSITDILVTEFGNNFINLSFLRLFRAARLIKLLRQGYTIRILLWTFVQSFKALPYVCLLIAMLFFIYAIIGMQVFGNIGIDVEDEDSDEDEFQITEHNNFRTFFQALMLLFRSATGEAWHNIMLSCLSGKPCDKNSGILTRECGNEFAYFYFVSFIFLCSFLMLNLFVAVIMDNFEYLTRDSSILGPHHLDEYVRVWAEYDPAACGRIHYKDMYSLLRVISPPLGLGKKCPHRVACKRLLRMDLPVADDNTVHFNSTLMALIRTALDIKIAKGGADKQQMDAELRKEMMAIWPNLSQKTLDLLVTPHKSTDLTVGKIYAAMMIMEYYRQSKAKKLQAMREEQDRTPLMFQRMEPPSPTQEGGPGQNALPSTQLDPGGALMAHESGLKESPSWVTQRAQEMFQKTGTWSPEQGPPTDMPNSQPNSQSVEMREMGRDGYSDSEHYLPMEGQGRAASMPRLPAENQRRRGRPRGNNLSTISDTSPMKRSASVLGPKARRLDDYSLERVPPEENQRHHQRRRDRSHRASERSLGRYTDVDTGLGTDLSMTTQSGDLPSKERDQERGRPKDRKHRQHHHHHHHHHHPPPPDKDRYAQERPDHGRARARDQRWSRSPSEGREHMAHRQ

SEQ ID NO: 11 - protein, 2505 aa, Homo sapiens
MARFGDEMPARYGGGGSGAAAGVVVGSGGGRGAGGSRQGGQPGAQRMYKQSMAQRARTMALYNPIPVRQNCLTVNRSLFLFSEDNVVRKYAKKITEWPPFEYMILATIIANCIVLALEQHLPDDDKTPMSERLDDTEPYFIGIFCFEAGIKIIALGFAFHKGSYLRNGWNVMDFVVVLTGILATVGTEFDLRTLRAVRVLRPLKLVSGIPSLQVVLKSIMKAMIPLLQIGLLLFFAILIFAIIGLEFYMGKFHTTCFEEGTDDIQGESPAPCGTEEPARTCPNGTKCQPYWEGPNNGITQFDNILFAVLTVFQCITMEGWTDLLYNSNDASGNTWNWLYFIPLIIIGSFFMLNLVLGVLSGEFAKERERVENRRAFLKLRRQQQIERELNGYMEWISKAEEVILAEDETDGEQRHPFDGALRRTTIKKSKTDLLNPEEAEDQLADIASVGSPFARASIKSAKLENSTFFHKKERRMRFYIRRMVKTQAFYWTVLSLVALNTLCVAIVHYNQPEWLSDFLYYAEFIFLGLFMSEMFIKMYGLGTRPYFHSSFNCFDCGVIIGSIFEVIWAVIKPGTSFGISVLRALRLLRIFKVTKYWASLRNLVVSLLNSMKSIISLLFLLFLFIVVFALLGMQLFGGQFNFDEGTPPTNFDTFPAAIMTVFQILTGEDWNEVMYDGIKSQGGVQGGMVFSIYFIVLTLFGNYTLLNVFLAIAVDNLANAQELTKDEQEEEEAANQKLALQKAKEVAEVSPLSAANMSIAVKEQQKNQKPAKSVWEQRTSEMRKQNLLASREALYNEMDPDERWKAAYTRHLRPDMKTHLDRPLVVDPQENRNNNTNKSRAAEPTVDQRLGQQRAEDFLRKQARYHDRARDPSGSAGLDARRPWAGSQEAELSREGPYGRESDHHAREGSLEQPGFWEGEAERGKAGDPHRRHVHRQGGSRESRSGSPRTGADGEHRRHRAHRRPGEEGPEDKAERRARHREGSRPARGGEGEGEGPDGGERRRRHRHGAPATYEGDARREDKERRHRRRKENQGSGVPVSGPNLSTTRPIQQDLGRQDPPLAEDIDNMKNNKLATAESAAPHGSLGHAGLPQSPAKMGNSTDPGPMLAIPAMATNPQNAASRRTPNNPGNPSNPGPPKTPENSLIVTNPSGTQTNSAKTARKPDHTTVDIPPACPPPLNHTVVQVNKNANPDPLPKKEEEKKEEEEDDRGEDGPKPMPPYSSMFILSTTNPLRRLCHYILNLRYFEMCILMVIAMSSIALAAEDPVQPNAPRNNVLRYFDYVFTGVFTFEMVIKMIDLGLVLHQGAYFRDLWNILDFIVVSGALVAFAFTGNSKGKDINTIKSLRVLRVLRPLKTIKRLPKLKAVFDCVVNSLKNVFNILIVYMLFMFIFAVVAVQLFKGKFFHCTDESKEFEKDCRGKYLLYEKNEVKARDREWKKYEFHYDNVLWALLTLFTVSTGEGWPQVLKHSVDATFENQGPSPGYRMEMSIFYVVYFVVFPFFFVNIFVALIIITFQEQGDKMMEEYSLEKNERACIDFAISAKPLTRHMPQNKQSFQYRMWQFVVSPPFEYTIMAMIALNTIVLMMKFYGASVAYENALRVFNIVFTSLFSLECVLKVMAFGILNYFRDAWNIFDFVTVLGSITDILVTEFGNNFINLSFLRLFRAARLIKLLRQGYTIRILLWTFVQSFKALPYVCLLIAMLFFIYAIIGMQVFGNIGIDVEDEDSDEDEFQITEHNNFRTFFQALMLLFRSATGEAWHNIMLSCLSGKPCDKNSGILTRECGNEFAYFYFVSFIFLCSFLMLNLFVAVIMDNFEYLTRDSSILGPHHLDEYVRVWAEYDPAACGRIHYKDMYSLLRVISPPLGLGKKCPHRVACKRLLRMDLPVADDNTVHFNSTLMALIRTALDIKIAKGGADKQQMDAELRKEMMAIWPNLSQKTLDLLVTPHKSTDLTVGKIYAAMMIMEYYRQSKAKKLQAMREEQDRTPLMFQRMEPPSPTQEGGPGQNALPSTQLDPGGALMAHESGLKESPSWVTQRAQEMFQKTGTWSPEQGPPTDMPNSQPNSQSVEMREMGRDGYSDSEHYLPMEGQGRAASMPRLPAENQRRRGRPRGNNLSTISDTSPMKRSASVLGPKARRLDDYSLERVPPEENQRHHQRRRDRSHRASERSLGRYTDVDTGLGTDLSMTTQSGDLPSKERDQERGRPKDRKHRQHHHHHHHHHHPPPPDKDRYAQERPDHGRARARDQRWSRSPSEGREHMAHRQGSSSVSGSPAPSTSGTSTPRRGRRQLPQTPSTPRPHVSYSPVIRKAGGSGPPQQQQQQQQQQQAVARPGRAATSGPRRYPGPTAEPLAGDRPPTGGHSSGRSPRMERRVPGPARSESPRACRHGGARWPASGPHVSEGPPGPRHHGYYRGSDYDEADGPGSGGGEEAMAGAYDAPPPVRHASSGATGRSPRTPRASGPACASPSRHGRRLPNGYYPAHGLARPRGPGSRKGLHEPYSESDDDWC

SEQ ID NO: 12 - protein, 2312 aa, Homo sapiens
MARFGEAVVARPGSGDGDSDQSRNRQGTPVPASGQAAAYKQTKAQRARTMALYNPIPVRQNCFTVNRSLFIFGEDNIVRKYAKKLIDWPPFEYMILATIIANCIVLALEQHLPEDDKTPMSRRLEKTEPYFIGIFCFEAGIKIVALGFIFHKGSYLRNGWNVMDFIVVLSGILATAGTHFNTHVDLRTLRAVRVLRPLKLVSGIPSLQIVLKSIMKAMVPLLQIGLLLFFAILMFAIIGLEFYSGKLHRACFMNNSGILEGFDPPHPCGVQGCPAGYECKDWIGPNDGITQFDNILFAVLTVFQCITMEGWTTVLYNTNDALGATWNWLYFIPLIIIGSFFVLNLVLGVLSGEFAKERERVENRRAFMKLRRQQQIERELNGYRAWIDKAEEVMLAEENKNAGTSALEVLRRATIKRSRTEAMTRDSSDEHCVDISSVGTPLARASIKSAKVDGVSYFRHKERLLRISIRHMVKSQVFYWIVLSLVALNTACVAIVHHNQPQWLTHLLYYAEFLFLGLFLLEMSLKMYGMGPRLYFHSSFNCFDFGVTVGSIFEVVWAIFRPGTSFGISVLRALRLLRIFKITKYWASLRNLVVSLMSSMKSIISLLFLLFLFIVVFALLGMQLFGGRFNFNDGTPSANFDTFPAAIITVFQILTGEDWNEVMYNGIRSQGGVSSGMWSAIYFIVLTLFGNYTLLNVFLAIAVDNLANAQELTKDEQEEEEAFNQKHALQKAKEVSPMSAPNMPSIERDRRRRHHMSMWEPRSSHLRERRRRHHMSVWEQRTSQLRKHMQMSSQEALNREEAPTMNPLNPLNPLSSLNPLNAHPSLYRRPRAIEGWPGLALEKFEEERISRGGSLKGDGGDRSSALDNQRTPLSLGQREPPWLARPCHGNCDPTQQEAGGGEAVVTFEDRARHRQSQRRSRHRRVRTEGKESSSASRSRSASQERSLDEAMPTEGEKDHELRGNHGAKEPTIQEERAQDLRRTNSLMVSRGSGLAGGLDEADTPLVLPHPELEVGKHVVLTEQEPEGSSEQALLGNVQLDMGRVISQSEPDLSCITANTDKATTESTSVTVAIPDVDPLVDSTVVHISNKTDGEASPLKEAEIREDEEEVEKKKQKKEKRETGKAMVPHSSMFIFSTTNPIRRACHYIVNLRYFEMCILLVIAASSIALAAEDPVLTNSERNKVLRYFDYVFTGVFTFEMVIKMIDQGLILQDGSYFRDLWNILDFVVVVGALVAFALANALGTNKGRDIKTIKSLRVLRVLRPLKTIKRLPKLKAVFDCVVTSLKNVFNILIVYKLFMFIFAVIAVQLFKGKFFYCTDSSKDTEKECIGNYVDHEKNKMEVKGREWKRHEFHYDNIIWALLTLFTVSTGEGWPQVLQHSVDVTEEDRGPSRSNRMEMSIFYVVYFVVFPFFFVNIFVALIIITFQEQGDKMMEECSLEKNERACIDFAISAKPLTRYMPQNRHTFQYRVWHFVVSPSFEYTIMAMIALNTVVLMMKYYSAPCTYELALKYLNIAFTMVFSLECVLKVIAFGFLNYFRDTWNIFDFITVIGSITEIILTDSKLVNTSGFNMSFLKLFRAARLIKLLRQGYTIRILLWTFVQSFKALPYVCLLIAMLFFIYAIIGMQVFGNIKLDEESHINRHNNFRSFFGSLMLLFRSATGEAWQEIMLSCLGEKGCEPDTTAPSGQNENERCGTDLAYVYFVSFIFFCSFLMLNLFVAVIMDNFEYLTRDSSILGPHHLDEFVRVWAEYDRAACGRIHYTEMYEMLTLMSPPLGLGKRCPSKVAYKRLVLMNMPVAEDMTVHFTSTLMALIRTALDIKIAKGGADRQQLDSELQKETLAIWPHLSQKMLDLLVPMPKASDLTVGKIYAAMMIMDYYKQSKVKKQRQQLEEQKNAPMFQRMEPSSLPQEIIANAKALPYLQQDPVSGLSGRSGYPSMSPLSPQDIFQLACMDPTDDGQFQERQSLEPEVSELKSVQPSNHGIYLPSDTQEHAGSGRASSMPRLTVDPQVVTDPSSMRRSFSTIRDKRSNSSWLEEFSMERSSENTYKSRRRSYHSSLRLSAHRLNSDSGHKSDTHRSGGRERGRSKERKHLLSPDVSRCNSEERGTQADWESPERRQSRSPSEGRSQTPNRQGTGSLSESSIPSVSDTSTPRRSRRQLPPVPPKPRPLLSYSSLIRHAGSISPPADGSEEGSPLTSQALESNNACLTESSNSPHPQQSQHASPQRYISEPYLALHEDSHASDCGEEETLTFEAAVATSLGRSNTIGSAPPLRHSWQMPNGHYRRRRRGGPGPGMMCGAVNNLLSDTEEDDKC

SEQ ID NO: 13 - protein, 2162 aa, Homo sapiens
PFEYMILATIIANCIVLALEQHLPEDDKTPMSRRLEKTEPYFIGIFCFEAGIKIVALGFIFHKGSYLRNGWNVMDFIVVLSGILATAGTHFNTHVDLRTLRAVRVLRPLKLVSGIPSLQIVLKSIMKAMVPLLQIGLLLFFAILMFAIIGLEFYSGKLHRACFMNNSGILEGFDPPHPCGVQGCPAGYECKDWIGPNDGITQFDNILFAVLTVFQCITMEGWTTVLYNTNDALGATWNWLYFIPLIIIGSFFVLNLVLGVLSGEFAKERERVENRRAFMKLRRQQQIERELNGYRAWIDKAEEVMLAEENKNAGTSALEVLRRATIKRSRTEAMTRDSSDEHCVDISSVGTPLARASIKSAKVDGVSYFRHKERLLRISIRHMVKSQVFYWIVLSLVALNTACVAIVHHNQPQWLTHLLYYAEFLFLGLFLLEMSLKMYGMGPRLYFHSSFNCFDFGVTVGSIFEVVWAIFRPGTSFGISVLRALRLLRIFKITKYWASLRNLVVSLMSSMKSIISLLFLLFLFIVVFALLGMQLFGGRFNFNDGTPSANFDTFPAAIMTVFQILTGEDWNEVMYNGIRSQGGVSSGMWSAIYFIVLTLFGNYTLLNVFLAIAVDNLANAQELTKDEQEEEEAFNQKHALQKAKEVSPMSAPNMPSIERERRRRHHMSVWEQRTSQLRKHMQMSSQEALNREEAPTMNPLNPLNPLSSLNPLNAHPSLYRRPRAIEGLALGLALEKFEEERISRGGSLKGDGGDRSSALDNQRTPLSLGQREPPWLARPCHGNCDPTQQEAGGGEAVVTFEDRARHRQSQRRSRHRRVRTEGKESSSASRSRSASQERSLDEAMPTEGEKDHELRGNHGAKEPTIQEERAQDLRRTNSLMVSRGSGLAGGLDEADTPLVLPHPELEVGKHVVLTEQEPEGSSEQALLGNVQLDMGRVISQSEPDLSCITANTDKATTESTSVTVAIPDVDPLVDSTVVHISNKTDGEASPLKEAEIREDEEEVEKKKQKKEKRETGKAMVPHSSMFIFSTTNPIRRACHYIVNLRYFEMCILLVIAASSIALAAEDPVLTNSERNKVLRYFDYVFTGVFTFEMVIKMIDQGLILQDGSYFRDLWNILDFVVVVGALVAFALANALGTNKGRDIKTIKSLRVLRVLRPLKTIKRLPKLKAVFDCVVTSLKNVFNILIVYKLFMFIFAVIAVQLFKGKFFYCTDSSKDTEKECIGNYVDHEKNKMEVKGREWKRHEFHYDNIIWALLTLFTVSTGEGWPQVLQHSVDVTEEDRGPSRSNRMEMSIFYVVYFVVFPFFFVNIFVALIIITFQEQGDKMMEECSLEKNERACIDFAISAKPLTRYMPQNRHTFQYRVWHFVVSPSFEYTIMAMIALNTVVLMMKYYSAPCTYELALKYLNIAFTMVFSLECVLKVIAFGFLNYFRDTWNIFDFITVIGSITEIILTDSKLVNTSGFNMSFLKLFRAARLIKLLRQGYTIRILLWTFVQSFKALPYVCLLIAMLFFIYAIIGMQVFGNIKLDEESHINRHNNFRSFFGSLMLLFRSATGEAWQEIMLSCLGEKGCEPDTTAPSGQNENERCGTDLAYVYFVSFIFFCSFLMLNLFVAVIMDNFEYLTRDSSILGPHHLDEFVRVWAEYDRAACGRIPYKDMYKLVRVISPPLGLGENCPYRVACKRLVLMNMPVAEDMTVHFTSTLMALIRTALDIKIAKGGADRQQLDSELQKETLAIWPHLSQKMLDLLVPMPKASDLTVGKIYAAMMIMDYYKQSKVKKQRQQLEEQKNAPMFQRMEPSSLPQEIIANAKALPYLQQDPVSGLSGRSGYPSMSPLSPQDIFQLACMDPADDGQFQERQSLVVTDPSSMRRSFSTIRDKRSNSSWLEEFSMERSSENTYKSRRRSYHSSLRLSAHRLNSDSGHKSDTHRSGGRERGRSKERKHLLSPDVSRCNSEERGTQADWESPERRQSRSPSEGRSQTPNRQGTGSLSESSIPSVSDTSTPRRSRRQLPPVPPKPRPLLSYSSLIRHAGSISPPADGSEEGSPLTSQALESNNACLTESSNSPHPQQSQHASPQRYISEPYLALHEDSHASDCGEEETLTFEAAVATSLGRSNTIGSAPPLRHSWQMPNGHYRRRRRGGPGPGMMCGAVNNLLSDTEEDDKC

SEQ ID NO: 14 - protein, 2224 aa, Homo sapiens
PFEYMILATIIANCIVLALEQHLPEDDKTPMSRRLEKTEPYFIGIFCFEAGIKIVALGFIFHKGSYLRNGWNVMDFIVVLSGILATAGTHFNTHVDLRTLRAVRVLRPLKLVSGIPSLQIVLKSIMKAMVPLLQIGLLLFFAILMFAIIGLEFYSGKLHRACFMNNSGILEGFDPPHPCGVQGCPAGYECKDWIGPNDGITQFDNILFAVLTVFQCITMEGWTTVLYNTNDALGATWNWLYFIPLIIIGSFFVLNLVLGVLSGEFAKERERVENRRAFMKLRRQQQIERELNGYRAWIDKAEEVMLAEENKNAGTSALEVLRRATIKRSRTEAMTRDSSDEHCVDISSVGTPLARASIKSAKVDGVSYFRHKERLLRISIRHMVKSQVFYWIVLSLVALNTACVAIVHHNQPQWLTHLLYYAEFLFLGLFLLEMSLKMYGMGPRLYFHSSFNCFDFGVTVGSIFEVVWAIFRPGTSFGISVLRALRLLRIFKITKYWASLRNLVVSLMSSMKSIISLLFLLFLFIVVFALLGMQLFGGRFNFNDGTPSANFDTFPAAIMTVFQILTGEDWNEVMYNGIRSQGGVSSGMWSAIYFIVLTLFGNYTLLNVFLAIAVDNLANAQELTKDEQEEEEAFNQKHALQKAKEVSPMSAPNMPSIERDRRRRHHMSMWEPRSSHLRERRRRHHMSVWEQRTSQLRKHMQMSSQEALNREEAPTMNPLNPLNPLSSLNPLNAHPSLYRRPRAIEGLALGLALEKFEEERISRGGSLKGDGGDRSSALDNQRTPLSLGQREPPWLARPCHGNCDPTQQEAGGGEAVVTFEDRARHRQSQRRSRHRRVRTEGKESSSASRSRSASQERSLDEAMPTEGEKDHELRGNHGAKEPTIQEERAQDLRRTNSLMVSRGSGLAGGLDEADTPLVLPHPELEVGKHVVLTEQEPEGSSEQALLGNVQLDMGRVISQSEPDLSCITANTDKATTESTSVTVAIPDVDPLVDSTVVHISNKTDGEASPLKEAEIREDEEEVEKKKQKKEKRETGKAMVPHSSMFIFSTTNPIRRACHYIVNLRYFEMCILLVIAASSIALAAEDPVLTNSERNKVLRYFDYVFTGVFTFEMVIKMIDQGLILQDGSYFRDLWNILDFVVVVGALVAFALANALGTNKGRDIKTIKSLRVLRVLRPLKTIKRLPKLKAVFDCVVTSLKNVFNILIVYKLFMFIFAVIAVQLFKGKFFYCTDSSKDTEKECIGNYVDHEKNKMEVKGREWKRHEFHYDNIIWALLTLFTVSTGEGWPQVLQHSVDVTEEDRGPSRSNRMEMSIFYVVYFVVFPFFFVNIFVALIIITFQEQGDKMMEECSLEKNERACIDFAISAKPLTRYMPQNRHTFQYRVWHFVVSPSFEYTIMAMIALNTVVLMMKYYSAPCTYELALKYLNIAFTMVFSLECVLKVIAFGFLNYFRDTWNIFDFITVIGSITEIILTDSKLVNTSGFNMSFLKLFRAARLIKLLRQGYTIRILLWTFVQSFKALPYVCLLIAMLFFIYAIIGMQVFGNIKLDEESHINRHNNFRSFFGSLMLLFRSATGEAWQEIMLSCLGEKGCEPDTTAPSGQNENERCGTDLAYVYFVSFIFFCSFLMLNLFVAVIMDNFEYLTRDSSILGPHHLDEFVRVWAEYDRAACGRIHYTEMYEMLTLMSPPLGLGKRCPSKVAYKRLVLMNMPVAEDMTVHFTSTLMALIRTALDIKIAKGGADRQQLDSELQKETLAIWPHLSQKMLDLLVPMPKASDLTVGKIYAAMMIMDYYKQSKVKKQRQQLEEQKNAPMFQRMEPSSLPQEIIANAKALPYLQQDPVSGLSGRSGYPSMSPLSPQDIFQLACMDPADDGQFQERQSLEPEVSELKSVQPSNHGIYLPSDTQEHAGSGRASSMPRLTVDPQVVTDPSSMRRSFSTIRDKRSNSSWLEEFSMERSSENTYKSRRRSYHSSLRLSAHRLNSDSGHKSDTHRSGGRERGRSKERKHLLSPDVSRCNSEERGTQADWESPERRQSRSPSEGRSQTPNRQGTGSLSESSIPSVSDTSTPRRSRRQLPPVPPKPRPLLSYSSLIRHAGSISPPADGSEEGSPLTSQALESNNACLTESSNSPHPQQSQHASPQRYISEPYLALHEDSHASDCGEEETLTFEAAVATSLGRSNTIGSAPPLRHSWQMPNGHYRRRRRGGPGPGMMCGAVNNLLSDTEEDDKC

SEQ ID NO: 15 - protein, 2377 aa, Homo sapiens
MDEEEDGAGAEESGQPRSFMRLNDLSGAGGRPGPGSAEKDPGSADSEAEGLPYPALAPVVFFYLSQDSRPRSWCLRTVCNPWFERISMLVILLNCVTLGMFRPCEDIACDSQRCRILQAFDDFIFAFFAVEMVVKMVALGIFGKKCYLGDTWNRLDFFIVIAGMLEYSLDLQNVSFSAVRTVRVLRPLRAINRVPSMRILVTLLLDTLPMLGNVLLLCFFVFFIFGIVGVQLWAGLLRNRCFLPENFSLPLSVDLERYYQTENEDESPFICSQPRENGMRSCRSVPTLRGDGGGGPPCGLDYEAYNSSSNTTCVNWNQYYTNCSAGEHNPFKGAINFDNIGYAWIAIFQVITLEGWVDIMYFVMDAHSFYNFIYFILLIIVGSFFMINLCLVVIATQFSETKQRESQLMREQRVRFLSNASTLASFSEPGSCYEELLKYLVYILRKAARRLAQVSRAAGVRVGLLSSPAPLGGQETQPSSSCSRSHRRLSVHHLVHHHHHHHHHYHLGNGTLRAPRASPEIQDRDANGSRRLMLPPPSTPALSGAPPGGAESVHSFYHADCHLEPVRCQAPPPRSPSEASGRTVGSGKVYPTVHTSPPPETLKEKALVEVAASSGPPTLTSLNIPPGPYSSMHKLLETQSTGACQSSCKISSPCLKADSGACGPDSCPYCARAGAGEVELADREMPDSDSEAVYEFTQDAQHSDLRDPHSRRQRSLGPDAEPSSVLAFWRLICDTFRKIVDSKYFGRGIMIAILVNTLSMGIEYHEQPEELTNALEISNIVFTSLFALEMLLKLLVYGPFGYIKNPYNIFDGVIVVISVWEIVGQQGGGLSVLRTFRLMRVLKLVRFLPALQRQLVVLMKTMDNVATFCMLLMLFIFIFSILGMHLFGCKFASERDGDTLPDRKNFDSLLWAIVTVFQILTQEDWNKVLYNGMASTSSWAALYFIALMTFGNYVLFNLLVAILVEGFQAEEISKREDASGQLSCIQLPVDSQGGDANKSESEPDFFSPSLDGDGDRKKCLALVSLGEHPELRKSLLPPLIIHTAATPMSLPKSTSTGLGEALGPASRRTSSSGSAEPGAAHEMKSPPSARSSPHSPWSAASSWTSRRSSRNSLGRAPSLKRRSPSGERRSLLSGEGQESQDEEESSEEERASPAGSDHRHRGSLEREAKSSFDLPDTLQVPGLHRTASGRGSASEHQDCNGKSASGRLARALRPDDPPLDGDDADDEGNLSKGERVRAWIRARLPACCLERDSWSAYIFPPQSRFRLLCHRIITHKMFDHVVLVIIFLNCITIAMERPKIDPHSAERIFLTLSNYIFTAVFLAEMTVKVVALGWCFGEQAYLRSSWNVLDGLLVLISVIDILVSMVSDSGTKILGMLRVLRLLRTLRPLRVISRAQGLKLVVETLMSSLKPIGNIVVICCAFFIIFGILGVQLFKGKFFVCQGEDTRNITNKSDCAEASYRWVRHKYNFDNLGQALMSLFVLASKDGWVDIMYDGLDAVGVDQQPIMNHNPWMLLYFISFLLIVAFFVLNMFVGVVVENFHKCRQHQEEEEARRREEKRLRRLEKKRRNLMLDDVIASGSSASAASEAQCKPYYSDYSRFRLLVHHLCTSHYLDLFITGVIGLNVVTMAMEHYQQPQILDEALKICNYIFTVIFVLESVFKLVAFGFRRFFQDRWNQLDLAIVLLSIMGITLEEIEVNASLPINPTIIRIMRVLRIARVLKLLKMAVGMRALLDTVMQALPQVGNLGLLFMLLFFIFAALGVELFGDLECDETHPCEGLGRHATFRNFGMAFLTLFRVSTGDNWNGIMKDTLRDCDQESTCYNTVISPIYFVSFVLTAQFVLVNVVIAVLMKHLEESNKEAKEEAELEAELELEMKTLSPQPHSPLGSPFLWPGVEGPDSPDSPKPGALHPAAHARSASHFSLEHPTDRQLFDTISLLIQGSLEWELKLMDELAGPGGQPSAFPSAPSLGGSDPQIPLAEMEALSLTSEIVSEPSCSLALTDDSLPDDMHTLLLSALESNMQPHPTELPGPDLLTVRKSGVSRTHSLPNDSYMCRHGSTAEGPLGHRGWGLPKAQSGSVLSVHSQPADTSYILQLPKDAPHLLQPHSAPTWGTIPKLPPPGRSPLAQRPLRRQAAIRTDSLDVQGLGSREDLLAEVSGPSPPLARAYSFWGQSSTQAQQHSRSHSKISKHMTPPAPCPGPEPNWGKGPPETRSSLELDTELSWISGDLLPPGGQEEPPSPRDLKKCYSVEAQSCQRRPTSWLDEQRRHSIAVSCLDSGSQPHLGTDPSNLGGQPLGGPGSRPKKKLSPPSITIDPPESQGPRTPPSPGICLRRRAPSSDSKDPLASGPPDSMAASPSPKKDVLSLSGLSSDPADLDP

SEQ ID NO: 16 - protein, 2250 aa, Homo sapiens
MDEEEDGAGAEESGQPRSFMRLNDLSGAGGRPGPGSAEKDPGSADSEAEGLPYPALAPVVFFYLSQDSRPRSWCLRTVCNPWFERISMLVILLNCVTLGMFRPCEDIACDSQRCRILQAFDDFIFAFFAVEMVVKMVALGIFGKKCYLGDTWNRLDFFIVIAGMLEYSLDLQNVSFSAVRTVRVLRPLRAINRVPSMRILVTLLLDTLPMLGNVLLLCFFVFFIFGIVGVQLWAGLLRNRCFLPENFSLPLSVDLERYYQTENEDESPFICSQPRENGMRSCRSVPTLRGDGGGGPPCGLDYEAYNSSSNTTCVNWNQYYTNCSAGEHNPFKGAINFDNIGYAWIAIFQVITLEGWVDIMYFVMDAHSFYNFIYFILLIIVGSFFMINLCLVVIATQFSETKQRESQLMREQRVRFLSNASTLASFSEPGSCYEELLKYLVYILRKAARRLAQVSRAAGVRVGLLSSPAPLGGQETQPSSSCSRSHRRLSVHHLVHHHHHHHHHYHLGNGTLRAPRASPEIQDRDANGSRRLMLPPPSTPALSGAPPGGAESVHSFYHADCHLEPVRCQAPPPRSPSEASGRTVGSGKVYPTVHTSPPPETLKEKALVEVAASSGPPTLTSLNIPPGPYSSMHKLLETQSTGACQSSCKISSPCLKADSGACGPDSCPYCARAGAGEVELADREMPDSDSEAVYEFTQDAQHSDLRDPHSRRQRSLGPDAEPSSVLAFWRLICDTFRKIVDSKYFGRGIMIAILVNTLSMGIEYHEQPEELTNALEISNIVFTSLFALEMLLKLLVYGPFGYIKNPYNIFDGVIVVISVWEIVGQQGGGLSVLRTFRLMRVLKLVRFLPALQRQLVVLMKTMDNVATFCMLLMLFIFIFSILGMHLFGCKFASERDGDTLPDRKNFDSLLWAIVTVFQILTQEDWNKVLYNGMASTSSWAALYFIALMTFGNYVLFNLLVAILVEGFQAEGDANKSESEPDFFSPSLDGDGDRKKCLALVSLGEHPELRKSLLPPLIIHTAATPMSLPKSTSTGLGEALGPASRRTSSSGSAEPGAAHEMKSPPSARSSPHSPWSAASSWTSRRSSRNSLGRAPSLKRRSPSGERRSLLSGEGQESQDEEESSEEERASPAGSDHRHRGSLEREAKSSFDLPDTLQVPGLHRTASGRGSASEHQDCNGKSASGRLARALRPDDPPLDGDDADDEGNLSKGERVRAWIRARLPACCLERDSWSAYIFPPQSRFRLLCHRIITHKMFDHVVLVIIFLNCITIAMERPKIDPHSAERIFLTLSNYIFTAVFLAEMTVKVVALGWCFGEQAYLRSSWNVLDGLLVLISVIDILVSMVSDSGTKILGMLRVLRLLRTLRPLRVISRAQGLKLVVETLMSSLKPIGNIVVICCAFFIIFGILGVQLFKGKFFVCQGEDTRNITNKSDCAEASYRWVRHKYNFDNLGQALMSLFVLASKDGWVDIMYDGLDAVGVDQQPIMNHNPWMLLYFISFLLIVAFFVLNMFVGVVVENFHKCRQHQEEEEARRREEKRLRRLEKKRRSKEKQMAEAQCKPYYSDYSRFRLLVHHLCTSHYLDLFITGVIGLNVVTMAMEHYQQPQILDEALKICNYIFTVIFVLESVFKLVAFGFRRFFQDRWNQLDLAIVLLSIMGITLEEIEVNASLPINPTIIRIMRVLRIARVLKLLKMAVGMRALLDTVMQALPQVGNLGLLFMLLFFIFAALGVELFGDLECDETHPCEGLGRHATFRNFGMAFLTLFRVSTGDNWNGIMKDTLRDCDQESTCYNTVISPIYFVSFVLTAQFVLVNVVIAVLMKHLEESNKEAKEEAELEAELELEMKTLSPQPHSPLGSPFLWPGVEGPDSPDSPKPGALHPAAHARSASHFSLEHPTMQPHPTELPGPDLLTVRKSGVSRTHSLPNDSYMCRHGSTAEGPLGHRGWGLPKAQSGSVLSVHSQPADTSYILQLPKDAPHLLQPHSAPTWGTIPKLPPPGRSPLAQRPLRRQAAIRTDSLDVQGLGSREDLLAEVSGPSPPLARAYSFWGQSSTQAQQHSRSHSKISKHMTPPAPCPGPEPNWGKGPPETRSSLELDTELSWISGDLLPPGGQEEPPSPRDLKKCYSVEAQSCQRRPTSWLDEQRRHSIAVSCLDSGSQPHLGTDPSNLGGQPLGGPGSRPKKKLSPPSITIDPPESQGPRTPPSPGICLRRRAPSSDSKDPLASGPPDSMAASPSPKKDVLSLSGLSSDPADLDP

SEQ ID NO: 17 - protein, 2243 aa, Homo sapiens
MDEEEDGAGAEESGQPRSFMRLNDLSGAGGRPGPGSAEKDPGSADSEAEGLPYPALAPVVFFYLSQDSRPRSWCLRTVCNPWFERISMLVILLNCVTLGMFRPCEDIACDSQRCRILQAFDDFIFAFFAVEMVVKMVALGIFGKKCYLGDTWNRLDFFIVIAGMLEYSLDLQNVSFSAVRTVRVLRPLRAINRVPSMRILVTLLLDTLPMLGNVLLLCFFVFFIFGIVGVQLWAGLLRNRCFLPENFSLPLSVDLERYYQTENEDESPFICSQPRENGMRSCRSVPTLRGDGGGGPPCGLDYEAYNSSSNTTCVNWNQYYTNCSAGEHNPFKGAINFDNIGYAWIAIFQVITLEGWVDIMYFVMDAHSFYNFIYFILLIIVGSFFMINLCLVVIATQFSETKQRESQLMREQRVRFLSNASTLASFSEPGSCYEELLKYLVYILRKAARRLAQVSRAAGVRVGLLSSPAPLGGQETQPSSSCSRSHRRLSVHHLVHHHHHHHHHYHLGNGTLRAPRASPEIQDRDANGSRRLMLPPPSTPALSGAPPGGAESVHSFYHADCHLEPVRCQAPPPRSPSEASGRTVGSGKVYPTVHTSPPPETLKEKALVEVAASSGPPTLTSLNIPPGPYSSMHKLLETQSTGACQSSCKISSPCLKADSGACGPDSCPYCARAGAGEVELADREMPDSDSEAVYEFTQDAQHSDLRDPHSRRQRSLGPDAEPSSVLAFWRLICDTFRKIVDSKYFGRGIMIAILVNTLSMGIEYHEQPEELTNALEISNIVFTSLFALEMLLKLLVYGPFGYIKNPYNIFDGVIVVISVWEIVGQQGGGLSVLRTFRLMRVLKLVRFLPALQRQLVVLMKTMDNVATFCMLLMLFIFIFSILGMHLFGCKFASERDGDTLPDRKNFDSLLWAIVTVFQILTQEDWNKVLYNGMASTSSWAALYFIALMTFGNYVLFNLLVAILVEGFQAEGDANKSESEPDFFSPSLDGDGDRKKCLALVSLGEHPELRKSLLPPLIIHTAATPMSLPKSTSTGLGEALGPASRRTSSSGSAEPGAAHEMKSPPSARSSPHSPWSAASSWTSRRSSRNSLGRAPSLKRRSPSGERRSLLSGEGQESQDEEESSEEERASPAGSDHRHRGSLEREAKSSFDLPDTLQVPGLHRTASGRGSASEHQDCNGKSASGRLARALRPDDPPLDGDDADDEGNLSKGERVRAWIRARLPACCLERDSWSAYIFPPQSRFRLLCHRIITHKMFDHVVLVIIFLNCITIAMERPKIDPHSAERIFLTLSNYIFTAVFLAEMTVKVVALGWCFGEQAYLRSSWNVLDGLLVLISVIDILVSMVSDSGTKILGMLRVLRLLRTLRPLRVISRAQGLKLVVETLMSSLKPIGNIVVICCAFFIIFGILGVQLFKGKFFVCQGEDTRNITNKSDCAEASYRWVRHKYNFDNLGQALMSLFVLASKDGWVDIMYDGLDAVGVDQQPIMNHNPWMLLYFISFLLIVAFFVLNMFVGVVVENFHKCRQHQEEEEARRREEKRLRRLEKKRRKAQCKPYYSDYSRFRLLVHHLCTSHYLDLFITGVIGLNVVTMAMEHYQQPQILDEALKICNYIFTVIFVLESVFKLVAFGFRRFFQDRWNQLDLAIVLLSIMGITLEEIEVNASLPINPTIIRIMRVLRIARVLKLLKMAVGMRALLDTVMQALPQVGNLGLLFMLLFFIFAALGVELFGDLECDETHPCEGLGRHATFRNFGMAFLTLFRVSTGDNWNGIMKDTLRDCDQESTCYNTVISPIYFVSFVLTAQFVLVNVVIAVLMKHLEESNKEAKEEAELEAELELEMKTLSPQPHSPLGSPFLWPGVEGPDSPDSPKPGALHPAAHARSASHFSLEHPTMQPHPTELPGPDLLTVRKSGVSRTHSLPNDSYMCRHGSTAEGPLGHRGWGLPKAQSGSVLSVHSQPADTSYILQLPKDAPHLLQPHSAPTWGTIPKLPPPGRSPLAQRPLRRQAAIRTDSLDVQGLGSREDLLAEVSGPSPPLARAYSFWGQSSTQAQQHSRSHSKISKHMTPPAPCPGPEPNWGKGPPETRSSLELDTELSWISGDLLPPGGQEEPPSPRDLKKCYSVEAQSCQRRPTSWLDEQRRHSIAVSCLDSGSQPHLGTDPSNLGGQPLGGPGSRPKKKLSPPSITIDPPESQGPRTPPSPGICLRRRAPSSDSKDPLASGPPDSMAASPSPKKDVLSLSGLSSDPADLDP

SEQ ID NO: 18 - protein, 1207 aa, Homo sapiens
MDEEEDGAGAEESGQPRSFMRLNDLSGAGGRPGPGSAEKDPGSADSEAEGLPYPALAPVVFFYLSQDSRPRSWCLRTVCNPWFERISMLVILLNCVTLGMFRPCEDIACDSQRCRILQAFDDFIFAFFAVEMVVKMVALGIFGKKCYLGDTWNRLDFFIVIAGMLEYSLDLQNVSFSAVRTVRVLRPLRAINRVPSMRILVTLLLDTLPMLGNVLLLCFFVFFIFGIVGVQLWAGLLRNRCFLPENFSLPLSVDLERYYQTENEDESPFICSQPRENGMRSCRSVPTLRGDGGGGPPCGLDYEAYNSSSNTTCVNWNQYYTNCSAGEHNPFKGAINFDNIGYAWIAIFQVITLEGWVDIMYFVMDAHSFYNFIYFILLIIVGSFFMINLCLVVIATQFSETKQRESQLMREQRVRFLSNASTLASFSEPGSCYEELLKYLVYILRKAARRLAQVSRAAGVRVGLLSSPAPLGGQETQPSSSCSRSHRRLSVHHLVHHHHHHHHHYHLGNGTLRAPRASPEIQDRDANGSRRLMLPPPSTPALSGAPPGGAESVHSFYHADCHLEPVRCQAPPPRSPSEASGRTVGSGKVYPTVHTSPPPETLKEKALVEVAASSGPPTLTSLNIPPGPYSSMHKLLETQSTGACQSSCKISSPCLKADSGACGPDSCPYCARAGAGEVELADREMPDSDSEAVYEFTQDAQHSDLRDPHSRRQRSLGPDAEPSSVLAFWRLICDTFRKIVDSKYFGRGIMIAILVNTLSMGIEYHEQPEELTNALEISNIVFTSLFALEMLLKLLVYGPFGYIKNPYNIFDGVIVVISVWEIVGQQGGGLSVLRTFRLMRVLKLVRFLPALQRQLVVLMKTMDNVATFCMLLMLFIFIFSILGMHLFGCKFASERDGDTLPDRKNFDSLLWAIVTVFQILTQEDWNKVLYNGMASTSSWAALYFIALMTFGNYVLFNLLVAILVEGFQAEGDANKSESEPDFFSPSLDGDGDRKKCLALVSLGEHPELRKSLLPPLIIHTAATPMSLPKSTSTGLGEALGPASRRTSSSGSAEPGAAHEMKSPPSARSSPHSPWSAASSWTSRRSSRNSLGRAPSLKRRSPSGERRSLLSGEGQESQDEEESSEEERASPAGSDHRHRGSLEREAKSSFDLPDTLQVPGLHRTASGRGSASEHQDCNGKSASGRLARALRPDDPPLDGDDADDEGNL

SEQ ID NO: 19 - protein, 2353 aa, Homo sapiens
MTEGARAADEVRVPLGAPPPGPAALVGASPESPGAPGREAERGSELGVSPSESPAAERGAELGADEEQRVPYPALAATVFFCLGQTTRPRSWCLRLVCNPWFEHVSMLVIMLNCVTLGMFRPCEDVECGSERCNILEAFDAFIFAFFAVEMVIKMVALGLFGQKCYLGDTWNRLDFFIVVAGMMEYSLDGHNVSLSAIRTVRVLRPLRAINRVPSMRILVTLLLDTLPMLGNVLLLCFFVFFIFGIVGVQLWAGLLRNRCFLDSAFVRNNNLTFLRPYYQTEEGEENPFICSSRRDNGMQKCSHIPGRRELRMPCTLGWEAYTQPQAEGVGAARNACINWNQYYNVCRSGDSNPHNGAINFDNIGYAWIAIFQVITLEGWVDIMYYVMDAHSFYNFIYFILLIIVGSFFMINLCLVVIATQFSETKQRESQLMREQRARHLSNDSTLASFSEPGSCYEELLKYVGHIFRKVKRRSLRLYARWQSRWRKKVDPSAVQGQGPGHRQRRAGRHTASVHHLVYHHHHHHHHHYHFSHGSPRRPGPEPGACDTRLVRAGAPPSPPSPGRGPPDAESVHSIYHADCHIEGPQERARVAHAAATAAASLRLATGLGTMNYPTILPSGVGSGKGSTSPGPKGKWAGGPPGTGGHGPLSLNSPDPYEKIPHVVGEHGLGQAPGHLSGLSVPCPLPSPPAGTLTCELKSCPYCTRALEDPEGELSGSESGDSDGRGVYEFTQDVRHGDRWDPTRPPRATDTPGPGPGSPQRRAQQRAAPGEPGWMGRLWVTFSGKLRRIVDSKYFSRGIMMAILVNTLSMGVEYHEQPEELTNALEISNIVFTSMFALEMLLKLLACGPLGYIRNPYNIFDGIIVVISVWEIVGQADGGLSVLRTFRLLRVLKLVRFLPALRRQLVVLVKTMDNVATFCTLLMLFIFIFSILGMHLFGCKFSLKTDTGDTVPDRKNFDSLLWAIVTVFQILTQEDWNVVLYNGMASTSSWAALYFVALMTFGNYVLFNLLVAILVEGFQAEGDANRSDTDEDKTSVHFEEDFHKLRELQTTELKMCSLAVTPNGHLEGRGSLSPPLIMCTAATPMPTPKSSPFLDAAPSLPDSRRGSSSSGDPPLGDQKPPASLRSSPCAPWGPSGAWSSRRSSWSSLGRAPSLKRRGQCGERESLLSGEGKGSTDDEAEDGRAAPGPRATPLRRAESLDPRPLRPAALPPTKCRDRDGQVVALPSDFFLRIDSHREDAAELDDDSEDSCCLRLHKVLEPYKPQWCRSREAWALYLFSPQNRFRVSCQKVITHKMFDHVVLVFIFLNCVTIALERPDIDPGSTERVFLSVSNYIFTAIFVAEMMVKVVALGLLSGEHAYLQSSWNLLDGLLVLVSLVDIVVAMASAGGAKILGVLRVLRLLRTLRPLRVISRAPGLKLVVETLISSLRPIGNIVLICCAFFIIFGILGVQLFKGKFYYCEGPDTRNISTKAQCRAAHYRWVRRKYNFDNLGQALMSLFVLSSKDGWVNIMYDGLDAVGVDQQPVQNHNPWMLLYFISFLLIVSFFVLNMFVGVVVENFHKCRQHQEAEEARRREEKRLRRLERRRRSTFPSPEAQRRPYYADYSPTRRSIHSLCTSHYLDLFITFIICVNVITMSMEHYNQPKSLDEALKYCNYVFTIVFVFEAALKLVAFGFRRFFKDRWNQLDLAIVLLSLMGITLEEIEMSAALPINPTIIRIMRVLRIARVLKLLKMATGMRALLDTVVQALPQVGNLGLLFMLLFFIYAALGVELFGRLECSEDNPCEGLSRHATFSNFGMAFLTLFRVSTGDNWNGIMKDTLRECSREDKHCLSYLPALSPVYFVTFVLVAQFVLVNVVVAVLMKHLEESNKEAREDAELDAEIELEMAQGPGSARRVDADRPPLPQESPGARDAPNLVARKVSVSRMLSLPNDSYMFRPVVPASAPHPRPLQEVEMETYGAGTPLGSVASVHSPPAESCASLQIPLAVSSPARSGEPLHALSPRGTARSPSLSRLLCRQEAVHTDSLEGKIDSPRDTLDPAEPGEKTPVRPVTQGGSLQSPPRSPRPASVRTRKHTFGQRCVSSRPAAPGGEEAEASDPADEEVSHITSSACPWQPTAEPHGPEASPVAGGERDLRRLYSVDAQGFLDKPGRADEQWRPSAELGSGEPGEAKAWGPEAEPALGARRKKKMSPPCISVEPPAEDEGSARPSAAEGGSTTLRRRTPSCEATPHRDSLEPTEGSGAGGDPAAKGERWGQASCRAEHLTVPSFAFEPLDLGVPSGDPFLDGSHSVTPESRASSSGAIVPLEPPESEPPMPVGDPPEKRRGLYLTVPQCPLEKPGSPSATPAPGGGADDPV

SEQ ID NO: 20 - protein, 1536 aa, Homo sapiens
PEELTNALEISNIVFTSMFALEMLLKLLACGPLGYIRNPYNIFDGIIVVISVWEIVGQADGGLSVLRTFRLLRVLKLVRFLPALRRQLVVLVKTMDNVATFCTLLMLFIFIFSILGMHLFGCKFSLKTDTGDTVPDRKNFDSLLWAIVTVFQILTQEDWNVVLYNGMASTSSWAALYFVALMTFGNYVLFNLLVAILVEGFQAEGDANRSDTDEDKTSVHFEEDFHKLRELQTTELKMCSLAVTPNGHLEGRGSLSPPLIMCTAATPMPTPKSSPFLDAAPSLPDSRRGSSSSGDPPLGDQKPPASLRSSPCAPWGPSGAWSSRRSSWSSLGRAPSLKRRGQCGERESLLSGEGKGSTDDEAEDGRAAPGPRATPLRRAESLDPRPLRPAALPPTKCRDRDGQVVALPSDFFLRIDSHREDAAELDDDSEDSCCLRLHKVLEPYKPQWCRSREAWALYLFSPQNRFRVSCQKVITHKMFDHVVLVFIFLNCVTIALERPDIDPGSTERVFLSVSNYIFTAIFVAEMMVKVVALGLLSGEHAYLQSSWNLLDGLLVLVSLVDIVVAMASAGGAKILGVLRVLRLLRTLRPLRVISRAPGLKLVVETLISSLRPIGNIVLICCAFFIIFGILGVQLFKGKFYYCEGPDTRNISTKAQCRAAHYRWVRRKYNFDNLGQALMSLFVLSSKDGWVNIMYDGLDAVGVDQQPVQNHNPWMLLYFISFLLIVSFFVLNMFVGVVVENFHKCRQHQEAEEARRREEKRLRRLERRRRSTFPSPEAQRRPYYADYSPTRRSIHSLCTSHYLDLFITFIICVNVITMSMEHYNQPKSLDEALKYCNYVFTIVFVFEAALKLVAFGFRRFFKDRWNQLDLAIVLLSLMGITLEEIEMSAALPINPTIIRIMRVLRIARVLKLLKMATGMRALLDTVVQALPQVGNLGLLFMLLFFIYAALGVELFGRLECSEDNPCEGLSRHATFSNFGMAFLTLFRVSTGDNWNGIMKDTLRECSREDKHCLSYLPALSPVYFVTFVLVAQFVLVNVVVAVLMKHLEESNKEAREDAELDAEIELEMAQGPGSARRVDADRPPLPQESPGARDAPNLVARKVSVSRMLSLPNDSYMFRPVVPASAPHPRPLQEVEMETYGAGTPLGSVASVHSPPAESCASLQIPLAVSSPARSGEPLHALSPRGTARSPSLSRLLCRQEAVHTDSLEGKIDSPRDTLDPAEPGEKTPVRPVTQGGSLQSPPRSPRPASVRTRKHTFGQHCVSSRPAAPGGEEAEASDPADEEVSHITSSACPWQPTAEPHGPEASPVAGGERDLRRLYSVDAQGFLDKPGRADEQWRPSAELGSGEPGEAKAWGPEAEPALGARRKKKMSPPCISVEPPAEDEGSARPSAAEGGSTTLRRRTPSCEATPHRDSLEPTEGSGAGGDPAAKGERWGQASCRAEHLTVPSFAFEPLDLGVPSGDPFLDGSHSVTPESRASSSGAIVPLEPPESEPPMPVGDPPEKRRGLYLTVPQCPLEKPGSPSATPAPGGGADDPV

SEQ ID NO: 21 - protein, 28 aa, Conus geographus
  MISC_FEATURE (10)..(10): Xaa = any amino acid
CKSXGSSCSXTSYNCCRSCNXYTKRCYX

SEQ ID NO: 22 - protein, 25 aa, Conus magus
CKGKGAKCSRLMYDCCTGSCRSGKC

SEQ ID NO: 23 - protein, 48 aa, Agelenopsis aperta
KKKCIAKDYGRCKWGGTPCCRGRGCICSIMGTNCECKPRLIMEGLGLA

SEQ ID NO: 24 - protein, 76 aa, Agelenopsis aperta
SCIDIGGDCDGEKDDCQCCRRNGYCSCYSLFGYLKSGCKCVVGTSAEFQGICRRKARQCYNSDPDKCESHNKPKRR

SEQ ID NO: 25 - protein, 29 aa, Conus magus
TRCKGKGAPCRKTMYDCCSGSCGRRGKCG

SEQ ID NO: 26 - protein, 41 aa, Hysterocrates gigas
GVDKAGCRYMFGGCSVNDDCCPRLGCHSLFSYCAWDLTFSD